(19) United States
(12) Patent Application Publication
(10) Pub. No.: US 2021/0309995 A1
(43) Pub. Date: Oct. 7, 2021

(54) HOMOLOGY-DIRECTED REPAIR TEMPLATE DESIGN AND DELIVERY TO EDIT HEMOGLOBIN-RELATED MUTATIONS

(71) Applicant: Seattle Children's Hospital (dba Seattle Children's Research Institute), Seattle, WA (US)

(72) Inventors: David J. Rawlings, Seattle, WA (US); Sowmya Pattabhi, Seattle, WA (US); Andrew M. Scharenberg, Seattle, WA (US); Kyle Jacoby, Seattle, WA (US)

(21) Appl. No.: 17/050,637

(22) PCT Filed: Apr. 24, 2019
(86) PCT No.: PCT/US2019/028861

§ 371 Date: Oct. 26, 2020

Related U.S. Application Data
(60) Provisional application No. 62/663,553, filed on Apr. 27, 2018.
(60) Provisional application No. 62/820,521, filed on Mar. 19, 2019.

(51) Int. Cl. C12N 15/11; C12N 9/22; C12N 15/90; C07K 14/805; C12N 15/86; A61K 35/17; A61K 38/46; A61K 31/7088; A61P 7/00

(52) U.S. Cl. CPC C12N 15/11; C12N 9/22; C12N 15/907; C07K 14/805; C12N 15/86; A61K 48/00; A61K 38/465; A61K 31/7088; A61P 7/00; C12N 2750/14143; C12N 2310/20; A61K 35/17

(57) ABSTRACT

Some embodiments of the methods and compositions provided herein relate to modifying hemoglobin loci, such as hemoglobin-related mutations including sickle cell mutations. Some embodiments relate to modification of a sickle cell mutation through introduction of a phosphodiester DNA strand break at the site of the sickle cell mutation.

### RELATED APPLICATIONS

This application is a U.S. National Phase application of PCT International Application Number PCT/US2019/028861, filed on Apr. 24, 2019, designating the United States of America and published in the English language, which is an International Application of and claims the benefit of priority to U.S. Provisional Application No. 62/663,553, filed on Apr. 27, 2018 and U.S. Provisional Application No. 62/820,521, filed on Mar. 19, 2019. The disclosures of the above-referenced applications are hereby expressly incorporated by reference in their entireties.

### REFERENCE TO SEQUENCE LISTING

The present application is being filed along with a Sequence Listing in electronic format. The Sequence Listing is provided as a file entitled SEQLISTINGSCRI194NP, created Jun. 15, 2021, which is approximately 160 Kb in size. The information in the electronic format of the Sequence Listing is incorporated herein by reference in its entirety.

### FIELD OF THE INVENTION

Some embodiments of the methods and compositions provided herein relate to modifying hemoglobin loci, such as hemoglobin-related mutations including sickle cell mutations. Some embodiments relate to modification of a sickle cell mutation through introduction of a phosphodiester DNA strand break at the site of the sickle cell mutation.

### BACKGROUND OF THE INVENTION

Sickle-cell disease (SCD) includes blood disorders such as sickle-cell anemia. In some cases, SCD results in an abnormality in the oxygen-carrying protein hemoglobin found in red blood cells. This may lead the red blood cells comprising a rigid, sickle-like shape, and/or anemia.

Endonuclease-based systems have rapidly become significant gene editing tools. Examples of endonuclease-based approaches for gene editing include systems comprising, without limitations, zinc finger nucleases (ZFNs), TAL effector nucleases (TALENs), meganucleases (such as MegaTALs), and CRISPR/Cas9. The need for more approaches to inhibit or treat SCD is manifest.

### SUMMARY

Embodiments of the methods and compositions provided herein relate to modifying hemoglobin loci, such as hemoglobin-related mutations including sickle cell mutations. Some embodiments relate to a nucleic acid for homology directed repair (HDR) of an HBB gene.

Some embodiments include a method for editing an HBB gene in a cell, comprising: (i) introducing a polynucleotide encoding a guide RNA (gRNA) into the cell, and (ii) introducing a template polynucleotide into the cell.

In some embodiments, the gRNA comprises a nucleic acid having at least 95% identity to the nucleotide sequence of any one of SEQ ID NOs:01-06. In some embodiments, gRNA comprises a nucleic acid having at least 95% identity to the nucleotide sequence of any one of SEQ ID NOs:07-12. In some embodiments, the gRNA comprises the nucleotide sequence of any one of SEQ ID NOs:01-06. In some embodiments, the gRNA comprises the nucleotide sequence of SEQ ID NO:01. In some embodiments, the gRNA comprises the nucleotide sequence of SEQ ID NO:07.

In some embodiments, introducing a polynucleotide encoding a gRNA into the cell comprises contacting the cell with a ribonucleoprotein (RNP) comprising a CAS9 protein and the polynucleotide encoding the gRNA. In some embodiments, the CAS9 protein and the polynucleotide encoding the gRNA have a ratio between 0.1:1 and 1:10. In some embodiments, the CAS9 protein and the polynucleotide encoding the gRNA have a ratio between 1:1 and 1:5. In some embodiments, the CAS9 protein and the polynucleotide encoding the gRNA have a ratio of about 1:2.5.

In some embodiments, the template polynucleotide encodes at least a portion of the HBB gene, or complement thereof. In some embodiments, the template polynucleotide encodes at least a portion of a wild-type HBB gene, or complement thereof. In some embodiments, the at least a portion of the HBB gene comprises exon 1 of the HBB gene.

In some embodiments, a viral vector comprises the template polynucleotide. In some embodiments, the vector is an adeno-associated viral (AAV) vector. In some embodiments, the vector is a self-complementary AAV (scAAV) vector. In some embodiments, the template polynucleotide comprises at least about 4 kb of the HBB gene.

In some embodiments, a single-stranded donor oligonucleotide (ssODN) comprises the template polynucleotide. In some embodiments, the ssODN comprises a nucleotide sequence having at least 95% identity to the nucleotide sequence of any one of SEQ ID NOs:64-72. In some embodiments, the ssODN comprises a nucleotide sequence any one of SEQ ID NOs:64-72.

In some embodiments, a double-stranded break is created in exon 1 of the HBB gene. In some embodiments, the double-stranded break is created adjacent to the sixth codon in exon 1 of the HBB gene.

In some embodiments, step (i) is performed before step (ii). In some embodiments, steps (i) and (ii) are performed simultaneously. In some embodiments, steps (i) and/or (ii) comprise performing nucleofection. In some embodiments, performing nucleofection comprises use of a LONZA system. In some embodiments, the system comprises use of a square wave pulse. In some embodiments, steps (i) and/or (ii) comprise contacting about 200,000 cells/20 μl nucleofection reaction, wherein the nucleofection reaction comprises the gRNA and/or the template polynucleotide.

In some embodiments, the cell is mammalian. In some embodiments, the cell is human. In some embodiments, the cell is a primary cell. In some embodiments, the cell is a hematopoietic stem cell (HSC). In some embodiments, the cell is a T cell or a B cell. In some embodiments, the cell is a CD34+ cell.

In some embodiments, the HBB gene has at least 95% identity with the nucleotide sequence of SEQ ID NO:37.

In some embodiments, the nucleic acid includes one or more of: a first sequence encoding an HBB gene; a second sequence encoding one or more guide RNA cleavage sites; and a third sequence encoding one or more nuclease binding sites.

In some embodiments, the HBB gene comprises the nucleic acid sequence set forth in SEQ ID NO: 37. In some embodiments, the second sequence comprises the nucleic acid sequence set forth in SEQ ID NO: 1. In some embodiments, the one or more nuclease binding sites comprises a forward and reverse transcription activator-like effector nuclease (TALEN) binding site. In some embodiments, the one or more nucleic binding sites is a clustered regularly interspaced short palindromic repeats (CRISPR) associated protein 9 (Cas9) binding site. Some embodiments include one or more enhancer elements. Some embodiments include homology arm sequences. Some embodiments include a nucleic acid sequence encoding a promoter.

Some embodiments relate to a vector for promoting HDR of HBB protein expression in a cell. In some embodiments, the vector includes one or more of: a first sequence encoding a HBB gene; a second sequence encoding one or more guide RNA cleavage sites; and a third sequence encoding one or more nuclease binding sites.

In some embodiments, the HBB gene comprises the nucleic acid sequence set forth in SEQ ID NO: 37. In some embodiments, the second sequence comprises the nucleic acid sequence set forth in SEQ ID NO: 1. In some embodiments, the one or more nuclease binding sites comprises a forward and reverse transcription activator-like effector nuclease (TALEN) binding site. In some embodiments, the one or more nucleic binding sites is a clustered regularly interspaced short palindromic repeats (CRISPR) associated protein 9 (Cas9) binding site. Some embodiments include one or more enhancer elements. In some embodiments, the vector is an adeno-associated viral vector (AAV). In some embodiments, the vector is a self-complementary AAV (scAAV). In some embodiments, the cell is a human cell. In some embodiments, the cell is a primary cell. In some embodiments, the cell is an autologous cell. In some embodiments, the cell is a T cell. In some embodiments, the cell is a hematopoietic stem cell (HSC). In some embodiments, the cell is a CD34⁺ HSC. Some embodiments relate to a system for promoting HDR of CD40L protein expression in a cell. In some embodiments, the system includes any vector of one or more of the above paragraphs, and a nucleic acid encoding a nuclease. In some embodiments, the nuclease is a TALEN nuclease. In some embodiments, the nuclease is a Cas nuclease. In some embodiments, the vector and nucleic acid are configured for co-delivery to the cell. In some embodiments, co-delivery to the cell modifies endogenous HBB locus. In some embodiments, the cell is a primary human hematopoietic cell.

Some embodiments relate to a cell for expressing HBB. In some embodiments, the cell includes a nucleic acid. In some embodiments, the nucleic acid includes one or more of: a first sequence encoding an HBB gene; a second sequence encoding a promoter; a third sequence encoding one or more guide RNA cleavage sites; and a fourth sequence encoding one or more nuclease binding sites.

In some embodiments, the nucleic acid is in a vector. In some embodiments, the vector is an AAV. In some embodiments, the AAV is a scAAV. In some embodiments, the cell is a human cell. In some embodiments, the cell is a primary cell. In some embodiments, the cell is an autologous cell. In some embodiments, the cell is a T cell. In some embodiments, the cell is a HSC. In some embodiments, the cell is a CD34⁺ HSC.

Some embodiments relate to a method of promoting HDR of an HBB gene in a subject in need thereof. In some embodiments, the method includes one or more of: administering to a subject any cell or vector of one or more of the above paragraphs; and administering to the subject a nuclease.

In some embodiments the nuclease is a TALEN nuclease. In some embodiments, the nuclease is a Cas nuclease. In some embodiments, the nuclease is co-administered to the subject with the cell or with the vector. In some embodiments, the cell is from the subject and, wherein the cell is genetically modified by introducing the nucleic acid or the vector of one or more of the above paragraphs, into the cell. In some embodiments, the administering is performed by adoptive cell transfer. In some embodiments, the cell is a human cell. In some embodiments, the cell is a primary cell. In some embodiments, the cell is an autologous cell. In some embodiments, the cell is a T cell. In some embodiments, the cell is a HSC. In some embodiments, the cell is a CD34⁺ HSC. In some embodiments, the subject is suffering from sickle cell disease. In some embodiments, promoting HDR results in one or more edits to the HBB gene. In some embodiments, the one or more edits to the HBB gene comprises a correction to a sickle cell mutation. In some embodiments, the sickle cell mutation comprises an E7V mutation.

Some embodiments relate to a method of treating, inhibiting, or ameliorating sickle cell disease (SCD) or disease symptoms associated with SCD in a subject in need thereof. In some embodiments, the method includes one or more of: administering to a subject the cell or vector of any one or more of the above paragraphs; administering to the subject a nuclease; and optionally identifying or selecting the subject as one that would benefit from receiving a therapy for SCD or disease symptoms associated with SCD and/or, optionally measuring an improvement in the progression of SCD or an improvement in a disease symptom associated with SCD in said subject.

In some embodiments, the nuclease is a TALEN nuclease. In some embodiments, the nuclease is a CRISPR/Cas nuclease. In some embodiments, the nuclease is co-administered to the subject with the cell or with the vector. In some embodiments, the cell is from the subject, wherein the cell is genetically modified by introducing the nucleic acid or vector of any one or more of the above paragraphs, into the cell. In some embodiments, the administering is performed by adoptive cell transfer. In some embodiments, the cell is a human cell. In some embodiments, the cell is a primary cell. In some embodiments, the cell is an autologous cell. In some embodiments, the cell is a T cell. In some embodiments, the cell is a HSC. In some embodiments, the cell is a CD34⁺ HSC. Some embodiments include engrafting the cell into a subject's bone marrow. In some embodiments, the cell is from a subject, and the cell is from the same subject as the bone marrow.

### BRIEF DESCRIPTION OF THE DRAWINGS

FIG. 1A, FIG. 1B and FIG. 1C depict graphical representations showing data showing efficient editing at the HBB locus with nucleases. Each of FIG. 1A, FIG. 1B and

FIG. 1C is a graph depicting a % INDELs in response to nucleases.

FIG. 2A, FIG. 2B, FIG. 2C, FIG. 2D, FIG. 2E, and FIG. 2F depict graphical representations showing a design for testing of rAAV6 delivery of deletional repair templates 1242, 1243, 1244, 1245, and related data.

FIG. 3A, FIG. 3B, FIG. 3C, FIG. 3D, FIG. 3E, and FIG. 3F include a depiction of a design for testing of rAAV6 delivery of non-deletional repair templates 1289, 1290, and related data.

FIG. 4A, FIG. 4B, FIG. 4C, and FIG. 4D include a depiction of a design for testing of rAAV6 delivery of human codon-optimized sickle introduction cassettes 1246, 1247, 1248, 1249, and related data.

FIG. 5A, FIG. 5B, FIG. 5C, FIG. 5D, FIG. 5E, and FIG. 5F include a depiction of a design for testing of rAAV6 delivery of sickle mutation introduction (GTC) 1314, and related data.

FIG. 6A, FIG. 6B and FIG. 6C include a depiction of a design for testing of rAAV6 delivery of sickle introduction repair template 1321, and related data.

FIG. 7A, FIG. 7B, FIG. 7C, and FIG. 7D include a depiction of a design for testing of ssODN introducing a sickle mutation (GTC change), and related data.

FIG. 8A, FIG. 8B, FIG. 8C, FIG. 8D, FIG. 8E, and FIG. 8F include a depiction of a design for testing of ssODN for sickle correction (CCC GAA change), and related data.

FIG. 9A, FIG. 9B and FIG. 9C include graphical data showing engraftment of edited human cells in the bone marrow of W41 mice at 12 weeks.

FIG. 10A depicts a schematic representation of the genomic HBB gene showing the location of sgRNA and TALEN binding sites. A nucleotide substitution from GAG (codon 6) to GTC or GTG changes the amino acid from glutamate to valine and causes SCD.

FIG. 10B depicts screening of TALEN mRNA (T) or candidate sgRNA (g1-g6, delivered as RNPs) to create DSBs at the HBB gene measured by TIDE/ICE (donor n=2-3).

FIG. 10C depicts optimizing cas9: sgRNA ratio to maximize editing efficiency in mPBSCs. NHEJ rates were analyzed by TIDE/ICE (Cas9: sgRNA ratio of 1:1 (40 pmol each), donor n=2 or ratio of 1:2.5 (20 pmol of Cas9 and 50 pmol of sgRNA, donor n=15).

FIG. 10D depicts an evaluation of on-target disruption at HBB and possible off-target disruption at HBD by Mi Seq analysis in mPBSCs using sgRNA-g1 delivered as RNP (donor n=7).

FIG. 10E depicts editing efficiency of sgRNA-g1 delivered as RNP using the NEON electroporation system (donor n=15) or the Lonza nucleofection system (donor n=3). All bar graphs show mean±SD. * p<0.05 ** p<0.01 *** p<0.001 ****, p<0.0001. p-value was calculated by comparing each sample mean with the respective control sample mean by 2way ANOVA with Dunnett's multiple comparison

FIG. 10F depicts an off-target analysis of top 5 off-target genes predicted by CCTop algorithm. Gel shows amplicons of top 5 off-target genes amplified from mock-treated (M) and sgRNA-g1 RNP-treated (RNP) samples evaluated by T7 endonuclease assay. (i) OT1: DENND3 (lane 1-2), (ii) OT2: MIR7974 (lane 3-4), (iii) OT3: LINC01206 (lane 5-6) (iv) OT4: HBD (lane 7-8) (v) OT5: TULP4 (lane 9-10) (vi) Target site: HBB (lane 11-12). Asterisks (*) represent cleaved bands. # represents a ghost band that does not match any of the potential cleavage fragments (313 bp and 143 bp for TULP4).

FIG. 10G depicts a TIDE/ICE sequencing analysis of top 5 off-target genes (i) OT1: DENND3 (ii) OT2: MIR7679 (iii) OT3: LINC01206 (iv) OT4: HBD (v) OT5: TULP4 (vi) Target site: HBB (n=2 experiments).

FIG. 11A depicts a schematic representation of rAAV6 cassettes designed to drive either a GTC (E6V) introducing a sickle mutation or a GAA (E6optE) introducing a codon optimized SNP change at codon 6 by HDR.

FIG. 11B depicts an experimental timeline for testing gene-editing with RNP and rAAV6 delivery followed by erythroid differentiation in mPBSCs.

FIG. 11C depicts a WT (%), HDR (%) measured by ddPCR and NHEJ (%) measured by TIDE/ICE sequencing, respectively, following electroporation with RNP alone, transduction with rAAV6 donor template alone, or co-delivery of RNP and GTC (E6V) rAAV6 donor template at the indicated concentrations (donor n=4).

FIG. 11D depicts an RP-HPLC analysis of erythroid cells to measure β-globin expression in cells treated with RNP only, rAAV6 only, or RNP plus GTC (E6V) rAAV6 treated cells (donor n=7).

FIG. 11E depicts a WT (%), HDR (%) measured by ddPCR and NHEJ (%) measured by TIDE/ICE sequencing, respectively, following electroporation with RNP alone, transduction with rAAV6 donor template alone, or co-delivery of RNP and GAA (E6optE) rAAV6 donor template at the indicated concentrations (1% rAAV6; donor n=3).

FIG. 11F depicts an RP-HPLC analysis of erythroid cells to measure β-globin expression in cells treated with RNP only, rAAV6 only, or RNP plus GAA (E6optE) rAAV6 treated cells (donor n=3). (βA=Adult globin, βS=Sickle globin, γG=Gamma 2, γA=Gamma 1). All bar graphs show mean±SD. * p<0.05, ** p<0.01, *** p<0.001, **** p<0.0001. p-value was calculated by comparing each sample mean of NHEJ (%), HDR (%), WT (%) or globin sub-type (%) with the respective NHEJ (%), HDR (%), WT (%) or globin sub-type (%) of the mock sample by 2way ANOVA with Dunnett's multiple comparison.

FIG. 11G depicts HDR and NHEJ outcomes measured by ddPCR and TIDE/ICE sequencing, respectively, following co-delivery of RNP and GTC (E6V) rAAV6 using either the Neon electroporation system (n=3) or the Lonza nucleofection system (n=1).

FIG. 11H depicts colony sequencing of samples edited with RNP and transduced with GTC (E6V) rAAV6 (n=5) using the neon electroporation.

FIG. 11I depicts viability of mPBSCs on day 2 post-electroporation and GTC (E6V) or GAA (E6optE) rAAV6 transduction.

FIG. 11J depicts an IEC of erythroid cells to determine globin tetramers in vitro in cells treated with rAAV6 alone and RNP plus GTC (E6V) rAAV6 (HbF: Fetal, HbA: Adult, HbA2: Minor adult, HbS: Sickle). All bar graphs show mean±SD. n represents the number of individual experiments. * p<0.05, ** p<0.01, *** p<0.001, ****, p<0.0001. p-value was calculated by comparing each sample mean with the respective control sample mean by 2way ANOVA with Dunnett's multiple comparison.

FIG. 11K depicts an RP-HPLC analysis of edited and differentiated erythroid cells. RP-HPLC chromatogram trace of Reference, Mock, rAAV6 alone and RNP plus GTC (E6V) rAAV6 (3%) transduced cells driving sickle globin expression (α=alpha, βA=adult, βS=Sickle, γG=Gamma 2, γA=Gamma 1). Vertical numbers are HPLC elution times. Lower trace shows sickle globin expression (red arrow).

FIG. 11L depicts a schematic representation of complex cDNA cassettes delivered as rAAV6 tested with sgRNA-g1 RNP. 1321 has HBG1 Δ13 promoter driving GTC change (E6V amino acid change) along with erythroid enhancers; HPFH-2 and HS-40. MND-GFP serves as a surrogate for HDR and has a reverse orientation with a SV40 polyadenylation sequence. 1322 has an identical design to 1321, but has a deletion (Δ−127, −71) to remove the HBB promoter. Experimental set up was similar to FIG. 11B.

FIG. 11M depicts GFP expression measured by flow cytometry 14 days post-electroporation and transduction of RNP-alone, rAAV6-alone and RNP along with rAAV6 transduction (1321 donor n=2, 1322 donor n=1).

FIG. 11N depicts an RP-HPLC analysis of erythroid cells to measure β-globin expression in cells treated with RNP only, rAAV6 only, or RNP plus GTC (E6V) 1321/1322 rAAV6 treated cells. All bar graphs show mean±SD. n represents the number of individual experiments. (α=alpha, βA=adult, βS=Sickle, γG=Gamma 2, γA=Gamma 1).

FIG. 11O depicts an RP-HPLC analysis of edited and differentiated erythroid cells. RP-HPLC chromatogram trace of Mock, rAAV6 alone, RNP alone and RNP plus GAA (E6optE) rAAV6 driving adult globin expression (α=alpha, βA=adult, βS=Sickle, γG=Gamma 2, γA=Gamma 1). Vertical numbers are HPLC elution times. Lower trace shows restoration of adult globin expression.

FIG. 12A depicts a schematic representation of ssODN cassette designed to drive either a GTC (E6V) introducing a sickle mutation or a GAA (E6optE) introducing a codon optimized SNP change at codon 6 by HDR.

FIG. 12B depicts an experimental timeline for testing gene-editing with RNP and ssODN delivery followed by erythroid differentiation in mPBSCs.

FIG. 12C depicts WT (%), HDR (%) measured by ddPCR and NHEJ (%) measured by TIDE/ICE sequencing respectively, following electroporation with RNP alone or co-delivery of RNP and GTC (E6V) ssODN donor template at the indicated concentrations (50 pmol of ssODN; donor n=5).

FIG. 12D depicts an RP-HPLC analysis of erythroid cells to measure β-globin expression in cells treated with RNP only or RNP plus GTC (E6V) ssODN treated cells (50 pmol of ssODN: donor n=5).

FIG. 12E depicts WT (%), HDR (%) measured by ddPCR and NHEJ (%) measured by TIDE/ICE sequencing respectively, following electroporation with RNP alone or co-delivery of RNP and GAA (E6optE) ssODN at the indicated concentrations (50 pmol of ssODN; donor n=8).

FIG. 12F depicts an RP-HPLC analysis of erythroid cells to measure β-globin expression in cells treated with RNP only or RNP plus GAA (E6optE) ssODN treated cells (50 pmol of ssODN; donor n=6). (α=alpha, βA=adult, βS=Sickle, γG=Gamma 2, γA=Gamma 1). All bar graphs show mean±SD. * p<0.05, ** p<0.01, *** p<0.001, **** p<0.0001. p-value was calculated by comparing each sample mean of NHEJ (%), HDR (%), WT (%) or globin sub-type (%) with the respective NHEJ (%), HDR (%), WT (%) or % globin sub-type (%) of the mock sample by 2way ANOVA with Dunnett's multiple comparison.

FIG. 12G depicts viability of CD34+mPBSCs on day 2 post-electroporation with GTC or GTG (E6V) ssODN introducing a sickle mutation or a GAA (E6optE) ssODN introducing a codon optimized SNP change at codon 6 by HDR.

FIG. 12H depicts WT (%), HDR (%) measured by ddPCR and NHEJ measured by TIDE/ICE sequencing respectively, following electroporation with RNP alone or co-delivery of RNP and donor GTG (E6V) ssODN at the indicated concentrations (50 pmol ssODN, donor n=3).

FIG. 12I depicts colony sequencing of samples edited with RNP and modified with GTG (E6V) ssODN and GAA (E6optE) ssODN tested with the Neon electroporation system (donor n=3).

FIG. 12J depicts an RP-HPLC analysis of erythroid cells to determine globin expression in edited cells with GTG (E6V) ssODN delivery (donor n=3). (α=alpha, βA=adult, βS=Sickle, γG=Gamma 2, γA=Gamma 1).

FIG. 12K depicts WT (%), HDR (%) measured by ddPCR and NHEJ (%) measured by TIDE/ICE sequencing respectively, following electroporation with RNP alone or co-delivery of RNP and GAA (E6optE) ssODN at the indicated concentrations using either the Neon electroporation system (50 pmol ssODN, n=2) or the Lonza nucleofection system (50 pmol ssODN, n=3). All bar graphs show mean±SD. n represents the number of individual experiments. * p<0.05, ** p<0.01, *** p<0.001, **** p<0.0001.

FIG. 12L depicts an RP-HPLC analysis of edited and differentiated erythroid cells. RP-HPLC chromatogram trace of Mock, RNP alone and RNP plus GTC (E6V) ssODN and rAAV6 donor templates driving sickle globin expression (α=alpha, βA=adult, βS=Sickle, γG=Gamma 2, γA=Gamma 1). Vertical numbers are HPLC elution times. Lower traces show sickle globin expression (arrow).

FIG. 12M depicts an RP-HPLC analysis of edited and differentiated erythroid cells. RP-HPLC chromatogram trace of Reference, Mock, RNP alone and RNP plus GTG (E6V) ssODN driving sickle globin expression (α=alpha, βA=adult, βS=Sickle, γG=Gamma 2, γA=Gamma 1). Vertical numbers are HPLC elution times. Lower trace shows sickle globin expression (arrow).

FIG. 12N depicts an RP-HPLC analysis of edited and differentiated erythroid cells. RP-HPLC chromatogram trace of Mock, RNP alone and RNP plus GAA (Eopt6E) ssODN and rAAV6 donor templates driving adult globin expression (α=alpha, βA=adult, βS=Sickle, γG=Gamma 2, γA=Gamma 1). Vertical numbers are HPLC elution times. Lower traces show restoration of adult globin expression.

FIG. 13A depicts quantification of HDR vs. NHEJ edits by MiSeq analysis in cells treated with GTC (E6V) rAAV6 (n=6) vs ssODNs (using GTC (E6V, n=8), GTG (E6V, n=3), or GAA (E6optE, n=2) ssODN).

FIG. 13B depicts an indel spectrum analysis by MiSeq comparing RNP-mediated editing alone to residual indels present in cells after promotion of HDR with either rAAV6 or ssODN delivery (donor n=6).

FIG. 13C depicts various gene editing outcomes WT, NHEJ (Insertion, substitution, deletion) and HDR measured in the following samples: Mock, RNP alone, co-delivery of RNP with rAAV6 and RNP with ssODN. The samples analyzed were the pre-transplant input samples analyzed on day 14 post-editing. (n=4). All bar graphs show mean±SD. * p<0.05, ** p<0.01, *** p<0.001, **** p<0.0001. p-value was calculated by comparing each sample mean of NHEJ (%), HDR (%) with the respective NHEJ (%), HDR (%) of the mock sample by 2way ANOVA with Dunnett's multiple comparison.

FIG. 13D depicts number of aligned paired end reads from in vitro edited samples and in vivo BM samples. Each dot represents a unique sample.

FIG. 13E depicts consensus sequences from predominant NHEJ events observed in Mock, RNP alone, co-delivery of RNP with rAAV6 and RNP with ssODN.

FIG. 13F depicts quantification of % frame shift mutations in vitro and in vivo by MiSeq analysis after promotion of HDR with either rAAV6 or ssODN delivery.

FIG. 14A depicts an experimental timeline for testing gene-editing with GTC (E6V) rAAV6 or ssODN treated cells in vitro in mPBSCs and in vivo in NBSGW mouse model.

FIG. 14B depicts human cell (hCD45+) chimerism in the BM and spleen with gating based upon FSC/SSC and single cells.

FIG. 14C depicts human CD19+ and CD33+ subsets within the BM hCD45+ population.

FIG. 14D depicts human CD235+ cells in the BM gated on mCD45-population. The BM cells were cultured ex vivo for 14 days in erythroid differentiation media and CD235+ (ex vivo) was measured by flow cytometry.

FIG. 14E depicts proportion of human CD34+ and CD34+CD38lo cells within the BM hCD45+ population.

FIG. 14F depicts HDR rates determined by ddPCR within the GTC (E6V) rAAV6 or ssODN treated input cells (day 14, n=4); and 3 weeks (day 21; n=2) post-transplant and 12-14 weeks (day 84-96; Mock: n=8, RNP+rAAV6: n=17, RNP+ssODN: n=18) post-transplant.

FIG. 14G depicts NHEJ rates determined by TIDE/ICE sequencing for GTC (E6V) rAAV6 or ssODN treated input cells (day 14), 3 weeks (day 21) post-transplant and 12-14 weeks (day 84-96) post-transplant.

FIG. 14H depicts HDR rates determined by MiSeq analysis for GTC (E6V) rAAV6 or ssODN treated cells at the indicated time points.

FIG. 14I depicts NHEJ rates determined by MiSeq analysis for: GTC (E6V) rAAV6 or ssODN treated cells at indicated time points. n represents samples or animals. Input n=4, All bar graphs show mean±SD. ns: not significant. * p<0.05, ** p<0.01, *** p<0.001, **** p<0.0001. p-value was calculated by comparing each sample mean of NHEJ (%), HDR (%) and WT (%) with the respective NHEJ (%), HDR (%) and WT (%) of the mock sample by 2way ANOVA with Dunnett's multiple comparison.

FIG. 14J depicts human CD19+ and CD33+ populations in the spleen gated from hCD45+ populations.

FIG. 14K depicts human CD3+ population in the BM and spleen gated from non-CD19+ and non-CD33+ cells.

FIG. 14L depicts representative flow plots of human cells (hCD45+) within the BM of NB SGW recipient mice transplanted with HSC edited with GTC (E6V) donors. Flow plots demonstrate multi-lineage engraftment including: CD19+, CD33+ and CD235+ cells within the BM of (i) Mock-edited, (ii) rAAV6-edited and (iii) ssODN-edited cells recipients. Gating strategy: Live, Single cells, hCD45+>CD19+CD33+. Erythroid cells were gated on mCD45− cells.

FIG. 14M (left panels) depicts representative flow plots of CD34+ and CD34+CD38lo cells pre-transplant showing: (i) Mock-edited, (ii) rAAV6-edited or (iii) ssODN-edited (modified with GTC (E6V) populations. Gating strategy: Live, Single cells, hCD45+>CD34+CD38+>CD90+CD133+. FIG. 14M (right panels) depicts representative flow plots of CD34+CD38lo cells using additional markers identify populations enriched for LT-HSC as identified by CD133+CD90+ double positive cells.

FIG. 14N depicts representative flow plots of CD34+ and CD34+CD38lo compartment from BM of NB SGW mice transplanted with: (i) Mock-edited, (ii) rAAV6-edited or (iii) ssODN-edited cells (GTC (E6V) donor constructs). Gating strategy: Live, Single cells, hCD45+>CD34+CD38+.

FIG. 14O depicts an RP-HPLC analysis to measure β-globin subtypes in erythroid cultures following gene editing of CD34+ mPBSCs using GTC (E6V) rAAV6 or ssODN delivery.

FIG. 14P depicts BM cells isolated at 12-14 weeks from recipient mice transplanted with mock (n=2), GTC (E6V)-edited rAAV6 (n=4) or ssODN (n=3) modified cells, expanded ex vivo in erythroid culture conditions for 2 weeks after harvest. RP-HPLC analysis was performed to measure globin subtypes expressed (α=alpha, βA=adult, βS=Sickle, γG=Gamma 2, γA=Gamma 1).

FIG. 14Q depicts ion exchange HPLC of single BFU-E colonies (generated from methocult cultures) to determine globin tetramers expressed following gene editing.

FIG. 14R depicts a summary of Ion exchange HPLC of single BFU-E colonies to measure globin tetramers expressed in gene edited cells (HbF: Fetal, HbA: Adult, HbA2: Minor adult, HbS: Sickle). All bar graphs show mean±SD. n represents the number of individual animals. * p<0.05, ** p<0.01, *** p<0.001, **** p<0.0001, p-value was calculated by comparing each sample mean with the respective sample mean of the mock or control sample by 2way ANOVA with Dunnett's multiple comparison.

FIG. 14S depicts IEC analysis of edited and differentiated erythroid colonies. Example of ion exchange HPLC of single BFU-E colonies from transplant 4 to measure globin tetramers (HbF: Fetal, HbA: Adult, HbA2: Minor adult, HbS: Sickle). Lower traces demonstrate sickle globin expression (red arrow) in single colonies derived from engrafted, GTC (E6V) ssODN-edited, HSC. Vertical numbers are HPLC elution times.

FIG. 14T depicts an IEC analysis of edited and differentiated erythroid colonies. Ion exchange HPLC of single BFU-E colonies from transplant 3 to determine globin tetramers (HbF: Fetal, HbA: Adult, HbA2: Minor adult, HbS: Sickle). Lower trace demonstrates sickle globin expression (red arrow) in a colony derived from engrafted, GTC (E6V) ssODN-edited, HSC. Vertical numbers are HPLC elution times.

FIG. 15A depicts graphs of percent viability post-editing by cell counts, % HDR by ddPCR, % NHEJ by ICE and LT-HSC compartment measured by flow cytometry (Gating strategy: Live, Single cells, CD34⁺CD38^{lo}>CD90⁺CD133⁺), for either CM149 or ER100 LONZA nucleofection methods, using either SCGM or SFEM-II media.

FIG. 15B depicts graphs of percent beta-like globins post-editing and HDR using rAAV6 or ssODN donor template for either CM149 or ER100 LONZA nucleofection methods, using either SCGM or SFEM-II media.

FIG. 16A depicts graphs for percent viability, for either CM149 or ER100 LONZA nucleofection methods, using SFEM-II media at various cell densities at the time of nucleofection.

FIG. 16B depicts graphs for HDR, for either CM149 or ER100 LONZA nucleofection methods, using SFEM-II media at various cell densities at the time of nucleofection.

FIG. 16C depicts graphs for NHEJ, for either CM149 or ER100 LONZA nucleofection methods, using SFEM-II media at various cell densities at the time of nucleofection.

FIG. 17A depicts graphs of percent viabilities for days 2-14 post editing for cells treated with EP, RNP, or RNP and ssODNs for various LONZA nucleofection protocols.

FIG. 17B depicts graphs of percent HDR, NHEJ and beta-like globin expression for cells treated with EP, RNP, or RNP and ssODNs for various LONZA nucleofection protocols.

FIG. 17C depicts a comparison of viability and HDR for cells subjected to various LONZA nucleofection protocols.

FIG. 18 depicts graphs of percent viability, HDR and NHEJ for cells subjected to DU100 or CX100 LONZA nucleofection protocols.

FIG. 19 shows ddPCR assay results for representative Mock, AAV, RNP, RNP+AAV and RNP+ssODN samples for both the E6V (GTC) change and EoptE (GAA) change.

FIG. 20A depicts a graph of percent HDR determined from ddPCR data.

FIG. 20B depicts a graph of percent HDR determined from ICE algorithm data.

### DETAILED DESCRIPTION

Some embodiments of the compositions and methods disclosed herein relate to editing hemoglobin-related mutations. Some such embodiments include in situ editing a sickle cell mutation through introduction of a phosphodiester DNA strand break at the site of the sickle cell mutation.

Sickle cell disease is caused by a single nucleotide transversion that increases the hydrophobicity of adult globin (βA) and renders it susceptible to polymerization. Patients with SCD are frequently transfusion-dependent with increased morbidity and a reduced life-span. While curative treatment can be achieved through HLA-matched allogeneic transplant from a healthy donor, the availability of HLA-matched donors is limited, and the outcomes are complicated by the possibility of graft-versus-host disease (GvHD) and short-term and long-term impacts following higher intensity myelo-ablative conditioning. Gene editing in autologous stem cells could circumvent the limitation of HLA-matched donor availability and directly correct the disease-causing mutation in self-renewing stem cells. Additionally, establishment of successful targeted-gene editing would mitigate the historical risk of random integration posed by early viral vectors.

Gene editing includes a site-specific endonuclease that creates a double-stranded break (DSB) that is resolved by cellular DNA repair machinery as seamless repair, error-prone non-homologous end joining (NHEJ), or precise HDR in the presence of a DNA donor template. These repair outcomes are markedly influenced by the stage of the cell cycle. DSBs in quiescent cells in G0/G1 phase are primarily resolved as NHEJ whereas resolution by HDR requires entry into S/G2 phase. These repair outcomes are mutually exclusive and therefore compete for overall outcome within individual HSC and across the HSC population.

The SCD single nucleotide mutation in exon 1 of the HBB gene can be corrected by homology-directed repair utilizing designer nucleases including zinc finger nuclease (ZFN) mRNA, TALENs and CRISPR/Cas9 in combination with several alternative methods for co-delivery of a DNA repair template including: integrase-defective lentiviral vectors (IDLV), rAAV6 and ssODN (Hoban, M D, et al. (2015). Blood 125: 2597-2604; DeWitt, Mass., et al (2017) Methods 121-122: 9-15; Dever, D P, et al. (2016) Nature 539: 384-389; and Hoban, M D, et al. (2016) Mol Ther 24: 1561-1569.)17-20, which are each hereby expressly incorporated by reference in their entireties). Of these approaches, using rAAV6 or single-stranded oligodeoxynucleotides (ssODN) comprise the most efficient donor template delivery platforms. However, total editing outcomes including frequency of precise HDR vs. NHEJ have not been simultaneously compared for rAAV6 and ssODN donor template delivery methods. In addition, to be clinically relevant and therapeutic, high-fidelity HDR outcomes should proportionately exceed the error prone NHEJ that improperly repairs DSBs and causes genomic instability.

To better understand the role of donor template delivery in: (i) the proportion of HDR and NHEJ outcomes; (ii) preserving the integrity and long-term engraftment potential of the HSC compartment after editing; and (iii) altering the longitudinal persistence of edited cells, different methods of donor template delivery in vitro and in vivo in adult CD34+ mPBSCs have been assessed as disclosed herein. These studies included a sickle mutation (GTC or GTG; encoding Glutamate to Valine change (E6V) or a silent change (GAA; encoding Glutamate to Glutamate (E6optE)), which were introduced into healthy donor mPBSCs. Following RNP-mediated disruption of exon 1 of HBB and alternative donor template delivery, the outcome of gene editing was assessed using molecular analysis via ddPCR and globin expression via induction of sickle globin (βS; in the case of GTC or GTG; E6V change) or restoration of adult globin (in the case of GAA change; E6optE) as a functional outcome. Using these approaches, the outcome of alternative delivery platforms was directly compared. The in vitro studies demonstrated superiority for rAAV6 delivery leading to proportionately greater HDR than NHEJ, whereas ssODN donor template delivery introduced significantly more NHEJ than HDR. In parallel, a longitudinal assessment of engraftment and persistence of transplanted HDR-edited HSC cells containing the GTC change (E6V) was performed. In contrast to the in vitro findings, a much greater percentage of cells modified by ssODN donor template persisted at 12-14 weeks in the bone marrow (BM) of NBSGW recipient mice. Taken together, the findings provide an important functional assessment of alternative methods for HDR-based gene editing.

The gene-editing systems and methods provided herein can be applied to any nuclease-based gene editing approach comprising, without limitations, gene disruption and/or gene targeting. For example, aspects of the present disclosure are related to CRISPR/Cas9-based gene editing. In some alternatives, Cas9 nuclease-mediated enhancement of gene editing is provided. In some embodiments, nuclease-based gene editing systems and methods are provided. Examples of nuclease-based approaches for gene editing include systems comprising nucleases such as, without limitations, ZFNs, TALENs, meganucleases (e.g., MegaTALs) or CRISPR/Cas9.

In some alternatives, nucleases perform targeted genome modification by introducing specific double stranded breaks at the desired locations in a genome and harness the cells mechanisms of repair to repair the induced break by homologous recombination and nonhomologous end-joining mechanism. Several engineered nucleases can be used. By way of example and not of limitation, nucleases can include zinc finger nucleases (ZFNs), Transcription Activator-like Effector Nucleases (TALENs), the CRISPR/Cas system, RNA guided endonucleases or engineered meganuclease re-engineered homing endonucleases. Targeted gene disruption has wide applicability for research, therapeutic, agricultural, and industrial uses. One strategy for producing targeted gene disruption is through the generation of double-strand DNA breaks caused by site-specific endonucleases.

In some alternatives, CRISPR/Cas9 enables the expression of guide RNAs efficiently in a wide variety of cell types. An example of a system for expressing guide RNAs is based on the use of adeno-associated virus vectors (AAV). AAV vectors are able to transduce a wide range of primary cells.

In some alternatives, Cas9-based approaches enhance gene editing efficiency with minimal toxicity when adeno-associated virus vectors (AAV) are used to express the guide RNA's necessary for Cas9 targeting.

### Definitions

As used herein, “a” or “an” may mean one or more than one.

As used herein, the term “about” indicates that a value includes the inherent variation of error for the method being employed to determine a value, or the variation that exists among experiments.

“Nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), or fragments generated by any of ligation, scission, endonuclease action, and exonuclease action. Nucleic acid molecules can be composed of monomers that are naturally-occurring nucleotides (such as DNA or RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, or azido groups, or sugars can be functionalized as ethers or esters. Moreover, the entire sugar moiety can be replaced with sterically and electronically similar structures, such as aza-sugars and/or carbocyclic sugar analogs. Examples of modifications in a base moiety include alkylated purines or pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded.

“Coding for” or “encoding” are used herein, and refer to the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

In some alternatives, the basic components of CRISPR/Cas9 system comprise a target gene, a guide RNA, and a Cas9 endonuclease, derivative, or fragment thereof. In some alternatives, one aspect of applying CRISPR/Cas9 for gene editing is the need for a system to deliver the guide RNAs efficiently to a wide variety of cell types. This could for example involve delivery of an in vitro generated guide RNA as a nucleic acid (the guide RNA generated by in vitro transcription or chemical synthesis). In some alternatives the nucleic acid encoding the guide RNA is rendered nuclease resistant by incorporation of modified bases, such as 2′O-methyl bases.

Exemplary guide RNAs useful with the alternatives described herein, which may contain one or more of the modified bases set forth herein are provided in sequences encoded by SEQ ID NO: 1, SEQ ID NO: 2, SEQ ID NO: 3, SEQ ID NO: 4, SEQ ID NO: 5 or SEQ ID NO: 6. In some alternatives, an important system for expressing guide RNAs is based on the use of adeno-associated virus (AAV) vectors because AAV vectors are able to transduce a wide range of primary cells. In some alternatives, AAV vectors do not cause infection and are not known to integrate into the genome. Therefore, in some alternatives, the use of AAV vectors has the benefits of being both safe and efficacious.

The term “complementary to” means that the complementary sequence is homologous to all or one or more portions of a reference polynucleotide sequence. For illustration, the nucleotide sequence “CATTAG” corresponds to a reference sequence “CATTAG” and is complementary to a reference sequence “GTAATC.”

A “promoter” is a nucleotide sequence that directs the transcription of a structural gene. In some alternatives, a promoter is located in the 5′ non-coding region of a gene, proximal to the transcriptional start site of a structural gene. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993); hereby expressly incorporated by reference in its entirety), cyclic AMP response elements (CREs), serum response elements (SREs; Treisman, Seminars in Cancer Biol. 1:47 (1990);); hereby expressly incorporated by reference in its entirety), glucocorticoid response elements (GREs), and binding sites for other transcription factors, such as CRE/ATF (O'Reilly et al., J. Biol. Chem. 267:19938 (1992)), AP2 (Ye et al., J. Biol. Chem. 269:25728 (1994)), SP1, cAMP response element binding protein (CREB; Loeken, Gene Expr. 3:253 (1993)) and octamer factors (see, in general, Watson et al., eds., Molecular Biology of the Gene, 4th ed. (The Benjamin/Cummings Publishing Company, Inc. 1987), and Lemaigre and Rousseau, Biochem. J. 303:1 (1994); all references); hereby expressly incorporated by reference in their entireties). As used herein, a promoter may be constitutively active, repressible or inducible. If a promoter is an inducible promoter, then the rate of transcription increases in response to an inducing agent. In contrast, the rate of transcription is not regulated by an inducing agent if the promoter is a constitutive promoter. Repressible promoters are also known. In some alternatives, a regulatory element can be an untranslated region. In some alternatives, an untranslated region is a 5′ untranslated region. In some alternatives, an untranslated region is a 3′ untranslated region. In some alternatives, either 5′ or 3′ untranslated region is used. In some alternatives, both 5′ and 3′ untranslated regions are used. One skilled in the art will understand the meaning of an untranslated region as used in the alternatives here.

A “regulatory element” is a nucleotide sequence that modulates the activity of a core promoter. For example, a regulatory element may contain a nucleotide sequence that binds with cellular factors enabling transcription exclusively or preferentially in particular cells, tissues, or organelles. These types of regulatory elements are normally associated with genes that are expressed in a “cell-specific,” “tissue-specific,” or “organelle-specific” manner. In some alternatives, a system for editing at least one target gene in a cell is provided, wherein the system comprises a first nucleic acid sequence encoding a CRISPR guide RNA, wherein the CRISPR guide RNA is complimentary to at least one target gene in a cell and, wherein said first nucleic acid sequence is present in a vector; said system also comprising a second nucleic acid sequence encoding a Cas9 protein, a third nucleic acid sequence encoding a first adenoviral protein, and a fourth nucleic acid sequence encoding a second adenoviral protein. In some alternatives, the first, second, third and fourth nucleic acid sequences are joined to regulatory elements that are operable in a eukaryotic cell, such as a human cell.

A “polypeptide” is a polymer of amino acid residues joined by peptide bonds, whether produced naturally or synthetically. Polypeptides of less than about 10 amino acid residues are commonly referred to as “peptides.” A polypeptide can be considered as a protein.

A “protein” is a macromolecule comprising one or more polypeptide chains. A protein may also comprise non-peptide components, such as carbohydrate groups. Carbohydrates and other non-peptide substituents may be added to a protein by the cell in which the protein is produced, and will vary with the type of cell. Proteins are defined herein in terms of their amino acid backbone structures; substituents such as carbohydrate groups are generally not specified, but may be present nonetheless. In some alternatives, a system for editing at least one target gene in a cell is provided, wherein the method comprises a first nucleic acid sequence encoding a CRISPR guide RNA, wherein the CRISPR guide RNA is complimentary to at least one target gene in a cell and, wherein said first nucleic acid sequence is present in a vector; said system also comprising a second nucleic acid sequence encoding a Cas9 protein, a third nucleic acid sequence encoding a first adenoviral protein and a fourth nucleic acid sequence encoding a second adenoviral protein.

By the term “host cell” is meant a cell that is introduced with Cas9-mRNA/AAV-guide RNA according to the present alternatives, as well as, cells that are provided with the systems herein. Host cells can be prokaryotic cells or eukaryotic cells. Examples of prokaryotic host cells include, but are not limited to E. coli, nitrogen fixing bacteria, Staphylococcus aureus, Staphylococcus albus, Lactobacillus acidophilus, Bacillus anthracis, Bacillus subtilis, Bacillus thuringiensis, Clostridium tetani, Clostridium botulinum, Streptococcus mutans, Streptococcus pneumoniae, mycoplasmas, or cyanobacteria. Examples of eukaryotic host cells include, but are not limited to, protozoa, fungi, algae, plant, insect, amphibian, avian and/or mammalian cells. In some alternatives, a system for editing at least one target gene in a cell is provided, wherein the cell is a eukaryotic cell. In some alternatives, the cell is a mammalian cell. In some alternatives, the cell is a human cell. In some alternatives, the cell is a primary cell. In some alternatives, the cell is not a transformed cell. In some alternatives, the cell is a primary lymphocyte. In some alternatives, the cell is a primary lymphocyte, a CD34+ stem cell, a hepatocyte, a cardiomyocyte, a neuron, a glial cell, a muscle cell or an intestinal cell.

The term “endonuclease” refers to enzymes that cleave the phosphodiester bond within a polynucleotide chain. The polynucleotide may be double-stranded DNA (dsDNA), single-stranded DNA (ssDNA), RNA, double-stranded hybrids of DNA and/or RNA, and/or synthetic DNA (for example, containing bases other than A, C, G, and T). An endonuclease may cut a polynucleotide symmetrically, leaving “blunt” ends, or in positions that are not directly opposing, creating overhangs, which may be referred to as “sticky ends.” The methods and compositions described herein may be applied to cleavage sites generated by endonucleases. In some alternatives of the system, the system can further provide nucleic acids that encode an endonuclease, such as Cas9, TALEN, or MegaTAL, or a fusion protein comprising a domain of an endonuclease, for example, Cas9, TALEN, or MegaTAL, or one or more portion thereof. These examples are not meant to be limiting and other endonucleases and alternatives of the system and methods comprising other endonucleases and variants and modifications of these exemplary alternatives are possible without undue experimentation. All such variations and modifications are within the scope of the current disclosure.

The term “transcription activator-like effector nuclease” or “TAL Effector Nuclease” (TALEN) refers to a nuclease comprising a TAL-effector domain fused to a nuclease domain. TAL-effector DNA binding domains, isolated from the plant pathogen Xanthomonas have been described (see Boch et al., (2009) Science 29 Oct. 2009 (10.1126/science.117881) and Moscou and Bogdanove, (2009) Science 29 Oct. 2009 (10.1126/science.1178817); both references are hereby expressly incorporated by reference in their entireties). These DNA binding domains may be engineered to bind to a desired target and fused to a nuclease domain, such as the Fok1 nuclease domain, to derive a TAL effector domain-nuclease fusion protein. The methods and systems described herein may be applied to cleavage sites generated by TAL effector nucleases. In some alternatives of the systems provided herein, the systems can further comprise a TALEN nuclease or a vector or nucleic acid encoding a TALEN nuclease. In some alternatives of the methods provided herein, the method can further comprise providing a nuclease, such as a TALEN nuclease.

In some alternatives, TALENS are artificial restriction enzymes generated by fusing a Tal effector DNA binding domain to a DNA cleavage domain. Tal effectors may be bacterial DNA-binding proteins consisting of highly homologous 34 amino-acid modules that can bind one nucleotide with high affinity. The variable twelfth and thirteenth amino acids of the TALENS module referred to as repeat-variable di-nucleotide, confers base specificity (i.e., NN→G/A, NI→A, NG→T, NK→G, HD→C, and NS→A/T/C/G) and TALEN arrays that can target a nucleotide sequence can be generated by assembling the individual modules. The relationship between the amino acid sequence and the DNA recognition has allowed engineering of specific DNA binding domains by the selecting of a combination of the repeat segments contacting the correlating Repeat Variable Diresidue (RVDs). TALENS can be used to edit genomes by inducing double-strand breaks (DSB) in the cells of interest, and in which the cells can respond with several types of repair mechanisms.

MegaTALs are derived from the combination of two distinct classes of DNA targeting enzymes. Meganucleases (also referred to as homing endonucleases) are single peptide chains that have the benefit of both DNA recognition and nuclease functions in the same domain. In some alternatives of the systems provided herein, the systems can further comprise a MegaTAL nuclease or a vector or nucleic acid encoding a MegaTAL nuclease. In some alternatives of the methods provided herein, the methods can further comprise providing MegaTAL nuclease or a vector or nucleic acid encoding a MegaTAL nuclease.

Zinc finger proteins (ZFP) are eukaryotic DNA binding proteins. The most common ZFP motifs for genome editing, for example, are the Cys2-His2 fingers, and each type are specific for a nucleotide triplet. Artificial ZFP domains can be generated to target specific DNA sequences that are usually 9-18 nt long by the assembly of individual zinc fingers. Zinc finger nucleases (ZFNs) are a powerful tool for performing targeted genomic manipulation in a variety of cell types in humans. ZFNs consist of an engineered DNA-binding zinc finger domain linked to a non-specific endonuclease domain and can introduce double-stranded breaks (DSBs) that stimulate both homologous and non-homologous recombination, which can then be harnessed to perform genomic manipulation. As such, ZFPs have potential in both research and gene therapy applications.

Clustered Regularly Interspaced Short Palindromic Repeats (CRISPR) comprise a DNA loci that can contain short repetitions of base sequences, in which each repetition is followed by short segments of spacer DNA from viral exposure. The CRISPR regions can be associated with cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and cut these exogenous genetic elements in a manner analogous to RNAi in eukaryotic organisms. As a genome editing mechanism, an RNA guided endonuclease, a Cas protein, and appropriate guide RNA can be delivered into a cell and the organisms' genome can be cut at a desired location. CRISPRS are an efficient mechanism for targeting/modifying genes and the mechanism is known to those skilled in the art.

Cas9 (CRISPR associated protein 9) is an RNA-guided DNA endonuclease enzyme associated with the CRISPR (Clustered Regularly Interspersed Palindromic Repeats) adaptive immunity system in Streptococcus pyogenes, among other bacteria. S. pyogenes utilizes Cas9 to memorize and later interrogate and cleave foreign DNA, such as invading bacteriophage DNA or plasmid DNA. Cas9 performs this interrogation by unwinding foreign DNA and checking for if it is complementary to the 20 base pair spacer region of the guide RNA. If the DNA substrate is complementary to the guide RNA, Cas9 cleaves the invading DNA.

CRISPRs (clustered regularly interspaced short palindromic repeats) are segments of prokaryotic DNA containing short repetitions of base sequences. Each repetition is followed by short segments of “spacer DNA” from previous exposures to a bacterial virus or plasmid. CRISPR/Cas system has been used for gene editing (adding, disrupting or changing the sequence of specific genes) and gene regulation in species throughout the tree of life. By delivering the Cas9 protein, a derivative, or fragment thereof and appropriate guide RNAs into a cell, the organism's genome can be cut at any desired location. It can be possible to use CRISPR to build RNA-guided gene drives capable of altering the genomes of entire populations. In some alternatives, a system for editing at least one target gene in a cell is provided, wherein the method comprises a first nucleic acid sequence encoding a CRISPR guide RNA, wherein the CRISPR guide RNA is complimentary to at least one target gene in a cell and, wherein said first nucleic acid sequence is present in a vector, a second nucleic acid sequence encoding a Cas9 protein, a derivative, or fragment thereof, a third nucleic acid sequence encoding a first adenoviral protein and a fourth nucleic acid sequence encoding a second adenoviral protein.

In some alternatives, the use of chemically modified guide RNAs is contemplated. Chemically-modified guide RNAs have been used in CRISPR-Cas genome editing in human primary cells (Hendel, A. et al., Nat Biotechnol. 2015 September; 33(9):985-9). Chemical modifications of guide RNAs can include modifications that confer nuclease resistance. Nucleases can be endonucleases, or exonucleases, or both. Some chemical modification, without limitations, include 2′-fluoro, 2′O-methyl, phosphorothioate dithiol 3′-3′ end linkage, 2-amino-dA, 5-mehtyl-dC, C-5 propynyl-C, or C-5 propynyl-U, morpholino. These examples are not meant to be limiting and other chemical modifications and variants and modifications of these exemplary alternatives are also contemplated.

The term “cleavage” refers to the breakage of the covalent backbone of a polynucleotide. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. Double stranded DNA, RNA, or DNA/RNA hybrid cleavage can result in the production of either blunt ends or staggered ends.

The term “subject” as used herein includes all members of the animal kingdom including non-human primates and humans. In some alternatives, a system for editing at least one target gene in a cell is provided, wherein the method comprises a first nucleic acid sequence encoding a CRISPR guide RNA, wherein the CRISPR guide RNA is complimentary to at least one target gene in a cell and, wherein said first nucleic acid sequence is present in a vector, a second nucleic acid sequence encoding a Cas9 protein, a derivative, or fragment thereof, a third nucleic acid sequence encoding a first adenoviral protein and a fourth nucleic acid sequence encoding a second adenoviral protein. In some alternatives, the cell that comprises an edited gene is delivered to a subject in need.

Targeted DNA double-strand breaks introduced by rare-cleaving endonucleases can be harnessed for gene disruption applications in diverse cell types by engaging non-homologous end joining DNA repair pathways. However, endonucleases create chemically clean breaks that are often subject to precise repair, limiting the efficiency of targeted gene disruption. Several alternatives described herein relate to a method of improving the rate of targeted gene disruptions caused by imprecise repair of endonuclease-induced site-specific DNA double-strand breaks. In some alternatives, systems can further comprise site specific endonucleases that are coupled with end-processing enzymes to enhance the rate of targeted gene disruption. Coupling may be, for example, physical, spatial, and/or temporal.

Not to be bound by any particular theory, the resolution of a double-strand DNA breaks by “error-prone” non-homologous end-joining (NHEJ) can be harnessed to create targeted disruptions and genetic knockouts, as the NHEJ process can result in insertions and deletions at the site of the break. NHEJ is mediated by several sub-pathways, each of which has distinct mutational consequences. The classical NHEJ pathway (cNHEJ) includes the KU/DNA-PKcs/Lig4/XRCC4 complex, and ligates ends back together with minimal processing. As the DNA breaks created by designer endonuclease platforms (zinc-finger nucleases (ZFNs), TAL effector nucleases (TALENs), and homing endonucleases (HEs)) all leave chemically clean, compatible overhang breaks that do not require processing prior to ligation, they are excellent substrates for precise repair by the cNHEJ pathway. In the absence or failure of the classical NHEJ pathway to resolve a break, alternative NHEJ pathways (altNHEJ) can substitute: however, these pathways are considerably more mutagenic.

Not to be bound by any particular theory, modification of DNA double-strand breaks by end-processing enzymes may bias repair towards an altNHEJ pathway. Further, different subsets of end-processing enzymes may enhance disruption by different mechanisms. For example, Trex2, an exonuclease that specifically hydrolyzes the phosphodiester bonds which are exposed at 3′ overhangs, biases repair at break sites toward mutagenic deletion. By contrast, terminal deoxynucleotidyl transferase (TdT), a non-templative polymerase, is expected to bias repair at break sites toward mutagenic insertions by promoting the addition of nucleotide bases to alter DNA ends prior to ligation. Accordingly, one of skill in the art can use end-processing enzymes with different activities to provide for a desired engineering outcome with any of the systems or methods provided herein. Further one of skill in the art may use the synergy between different end-processing enzymes so as to achieve maximal or unique types of effects.

A variety of RNA molecules encoding the endonucleases described herein, end-processing enzymes and fusion proteins may be constructed for providing the selected proteins or peptides to a cell. The RNA molecules encoding the endonucleases, end-processing enzyme, and fusion proteins may be modified to contain different codons to optimize expression in a selected host cell, as is known in the art. In some alternatives, the RNA can comprise a poly(A) tail of 50, 100, 150, 200, 250, 300, 350, 400, 450, or 500 covalently linked adenosine residues, or an amount of residues within a range defined by any two of the aforementioned values.

Several alternatives of the system further comprise a vector or nucleic acid for the simultaneous expression of a site-specific endonuclease and an end-processing enzyme to improve the efficiency of targeted gene disruption by up to ^{˜}70 fold, essentially fixing a mutagenic outcome in 100% of a population of cells containing the target site in less than 72 hours.

### Expression Vectors

In some alternatives, expression constructs can be designed using methods known in the art. Examples of nucleic acid expression vectors include, but are not limited to: recombinant viruses, lentiviruses, adenoviruses, plasmids, bacterial artificial chromosomes, yeast artificial chromosomes, human artificial chromosomes, minicircle DNA, episomes, cDNA, RNA, or PCR products. In some alternatives, nucleic acid expression vectors encode a single peptide (e.g., an endonuclease, an end-processing enzyme, or a fusion protein having endonuclease and end-processing activity). In some alternatives, nucleic acid expression vectors encode one or more endonucleases and one or more end-processing enzymes in a single, polycistronic expression cassette. In some alternatives of the system, one or more endonucleases and one or more end-processing enzymes are provided, wherein they are linked to each other by a 2A peptide sequence or an “autocleavage” or self-cleavage sequence. In some alternatives, the nucleic acid expression vectors are DNA expression vectors. In some alternatives, the nucleic acid expression vectors are RNA expression vectors. In some alternatives, the expression vectors are viral vectors. In some alternatives of the systems provided herein, the viral vector is an Adeno-associated virus (AAV) vector.

In some alternatives, a nucleic acid expression vector further comprises one or more selection markers that facilitate identification or selection of host cells that have received and express the endonuclease(s), end-processing enzyme(s), and/or fusion protein(s) having endonuclease and end-processing activity along with the selection marker. Examples of selection markers include, but are not limited to, genes encoding fluorescent proteins, e.g., EGFP, DS-Red, YFP, or CFP; genes encoding proteins conferring resistance to a selection agent, e.g., PuroR gene, ZeoR gene, HygroR gene, neoR gene, or the blasticidin resistance gene. In some cases, the selection marker comprises a fluorescent reporter and a selection marker.

In some alternatives, a DNA expression vector comprises a promoter capable of driving expression of one or more endonuclease(s), end-processing enzyme(s), and/or fusion protein(s) having endonuclease and end-processing activity. Examples of promoters include, but are not limited to, retroviral LTR elements; constitutive promoters such as CMV, HSV1-TK, SV40, EF-1α, or β-actin; inducible promoters, such as those containing Tet-operator elements; and/or tissue specific promoters. Suitable bacterial and eukaryotic promoters are well known in the art and described, e.g., in Sambrook et al., Molecular Cloning, A Laboratory Manual (2nd ed. 2001); Kriegler, Gene Transfer and Expression: A Laboratory Manual (1990); and Current Protocols in Molecular Biology (2010), the references are); hereby expressly incorporated by reference in their entireties. Non-limiting examples of plant promoters include promoter sequences derived from A. thaliana ubiquitin-3 (ubi-3).

In some alternatives, a nucleic acid encoding one or more endonucleases, end-processing enzymes, and/or fusion proteins having endonuclease and end-processing activity or exonuclease activity are cloned into a vector for transformation into eukaryotic cells along with the vectors and nucleic acid of the systems provided herein. In some alternatives, nucleic acids encoding different endonucleases and end-processing enzymes are cloned into the same vector. In such cases, the nucleic acids encoding different endonucleases and end-processing enzymes may optionally be separated by T2A, self-cleavage sequences, protease cleavage sites, or IRES sequences. Vectors can be prokaryotic vectors, e.g., plasmids, or shuttle vectors, insect vectors, or eukaryotic vectors, including plant vectors described herein. Expression of the nucleases and fusion proteins may be under the control of a constitutive promoter or an inducible promoter. In some alternatives, the vector comprises a nucleic acid sequence that encodes Cas9, a derivative, or fragment thereof. In some alternatives, the vector comprises a nucleic acid sequence that encodes Trex. In some alternatives, the genes and/or nucleic acids in the vector are codon optimized for expression in a mammalian cell, such as a human cell. In some alternatives, the vector is an mRNA. In some alternatives, the vector is an mRNA encoding a Cas9 protein, a derivative, or fragment thereof. In some alternatives, the nucleic acid encoding Cas9 protein, a derivative, or fragment thereof is codon optimized for expression in a eukaryotic cell, such as a human cell. In some alternatives, the Cas9 protein, a derivative, or fragment thereof is from S. pyogenes or is a consensus sequence made from other Cas9 proteins from other organisms.

Introduction of polypeptides having endonuclease and/or end-processing activity and/or polynucleotides encoding polypeptides having endonuclease and/or end-processing activity into host cells may use any suitable methods for nucleic acid or protein delivery as described herein or as would be known to one of ordinary skill in the art. The polypeptides and polynucleotides described herein can be delivered into cultured cells in vitro, as well as in situ into tissues and whole organisms. Introduction of the polypeptides and polynucleotides of the present alternatives into a host cell can be accomplished chemically, biologically, or mechanically. This may include, but is not limited to, electroporation, sonoporation, use of a gene gun, lipotransfection, calcium phosphate transfection, use of dendrimers, microinjection, polybrene, protoplast fusion, the use of viral vectors including adenoviral, AAV, or retroviral vectors, or group II ribozymes.

### Immune Response Against AAV Vectors

Adeno-associated viral (AAV) vectors may be used for gene therapy-based treatment genetic diseases. However, generation of immune responses against the AAV vector may undermine the therapeutic efficacy of the vector. Similarly, generation of immune responses against the AAV vector used in CRISPR/Cas9-based (or one or more other nucleases-based) genome editing might undermine the efficacy of gene targeting.

In some alternatives, it is contemplated that the AAV vectors used for CRISPR/Cas9-based (and/or one or more other nucleases-based) genome editing will possess reduced immunogenicity. In some alternatives, it is contemplated that the AAV vectors used for CRISPR/Cas9-based (and/or one or more other nucleases-based) genome editing will possess no immunogenicity. In some alternatives, because of the reduced immunogenicity, the likelihood of development of resistance against the AAV vector will be minimal. In some alternatives, because of the lack of immunogenicity, the likelihood of development of resistance against the AAV vector will be reduced or non-existent.

### Organisms

The alternatives described herein may be applicable to any eukaryotic organism in which it is desired to edit a gene, particularly, for example, a hemoglobin or hemoglobin-related gene. Examples of eukaryotic organisms include, but are not limited to, algae, plants, animals (e.g., mammals such as mice, rats, primates, pigs, cows, sheep, rabbits, dogs, cats, or horses etc.), fish, or insects. In some alternatives, isolated cells from the organism are genetically modified as described herein. In some alternatives, the modified cells develop into reproductively mature organisms. Eukaryotic (e.g., algae, yeast, plant, fungal, piscine, avian, or mammalian cells) cells can be used. Cells from organisms containing one or more additional genetic modifications can also be used.

Examples of mammalian cells include any cell or cell line of the organism of interest, for example oocytes, somatic cells, K562 cells, CHO (Chinese hamster ovary) cells, HEP-G2 cells, BaF-3 cells, Schneider cells, COS cells (monkey kidney cells expressing SV40 T-antigen), CV-1 cells, HuTu80 cells, NTERA2 cells, NB4 cells, HL-60 cells or HeLa cells, 293 cells or myeloma cells like SP2 or NSO. Peripheral blood mononucleocytes (PBMCs) or T-cells can also be used, as can embryonic and adult stem cells. For example, stem cells that can be used include embryonic stem cells (ES), induced pluripotent stem cells (iPSC), mesenchymal stem cells, hematopoietic stem cells, muscle stem cells, skin stem cells, adipose derived stem cells, or neuronal stem cells. In some alternatives, a system for editing at least one target gene in a cell is provided, wherein the system comprises a first nucleic acid sequence encoding a CRISPR guide RNA, wherein the CRISPR guide RNA is complimentary to at least one target gene in a cell and, wherein said first nucleic acid sequence is present in a vector, wherein said system further comprises a second nucleic acid sequence encoding a Cas9 protein, a derivative, or fragment thereof, a third nucleic acid sequence encoding a first adenoviral protein and a fourth nucleic acid sequence encoding a second adenoviral protein. In some alternatives, the cell is a eukaryotic cell. In some alternatives, the cell is a mammalian cell, such as a human cell. In some alternatives, the cell is a primary cell. In some alternatives the cell is not a transformed cell. In some alternatives, the cell is a primary lymphocyte, a CD34+ stem cell, a hepatocyte, a cardiomyocyte, a neuron, a glial cell, a muscle cell or an intestinal cell.

“Hematopoietic stem cells” or “HSC” as described herein, are precursor cells that can give rise to myeloid cells such as, for example, macrophages, monocytes, macrophages, neutrophils, basophils, eosinophils, erythrocytes, megakaryocytes/platelets, dendritic cells or lymphoid lineages (such as, for example, T-cells, B-cells, or NK-cells). In some alternatives, HSCs have a heterogeneous population in which three classes of stem cells exist, which are distinguished by their ratio of lymphoid to myeloid progeny in the blood (L/M).

### Pharmaceutical Administration

Cells manufactured by the systems or methods provided herein can be administered directly to a patient for targeted cleavage of a DNA sequence and for therapeutic or prophylactic applications, for example, for treating, inhibiting, or ameliorating a hemoglobin-related disease such as sickle cell disease or Beta thalassemia. In some alternatives, cells are manufactured by the compositions, systems or methods provided herein. In some alternatives, a composition is provided, wherein the composition comprises the cell. In some alternatives, the compositions described herein, can be used in methods of treating, preventing, ameliorating, or inhibiting a disease or ameliorating a disease condition or symptom associated with a disease. In some alternatives, the cells or compositions are administered to treat, prevent, ameliorate, or inhibit a genetic disease.

The compositions comprising the cells are administered in any suitable manner, and in some alternatives with pharmaceutically acceptable carriers. Suitable methods of administering such proteins or polynucleotides are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions that are available (see, e.g., Remington's Pharmaceutical Sciences).

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, or solutes that render the formulation isotonic with the blood of the intended recipient, or aqueous or non-aqueous sterile suspensions that can include suspending agents, solubilizers, thickening agents, stabilizers, or preservatives. The disclosed compositions can be administered, for example, by intravenous infusion, orally, topically, intraperitoneally, intravesically or intrathecally. The formulations of compounds can be presented in unit-dose or multi-dose sealed containers, such as ampules or vials. Injection solutions and suspensions can be prepared from sterile powders, granules, or tablets.

In some alternatives, one or more of parenteral, subcutaneous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal routes of administration are contemplated. In some alternatives, the composition to be administered can be formulated for delivery via one or more of the above noted routes.

### Nucleic Acid Compositions

Some alternatives relate to a composition for editing an HBB gene. In some alternatives, the composition comprises a nucleic acid. In some alternatives, the nucleic acid includes a single guide RNA (sgRNA) such as one that is encoded by any one of SEQ ID NOS: 1, 2, 3, 4, 5 or 6. In some alternatives, the nucleotide sequence encoding the sgRNA is at least 85%, 86%, 87%, 88%, 89%, 90%, 91%, 92%, 93%, 94%, 95%, 96%, 97%, 98%, 99% or 100% identical, or within a range defined by any two of the aforementioned percentages, to a sequence of SEQ ID NOS: 1, 2, 3, 4, 5 or 6.

In some alternatives, the nucleic acid includes a sgRNA combined with a guide RNA (gRNA) scaffold, such as one that is encoded by any one of SEQ ID NOS: 7, 8, 9, 10, 11 or 12. In some alternatives, the nucleotide sequence encoding the sgRNA is at least 85%, 86%, 87%, 88%, 89%, 90%, 91%, 92%, 93%, 94%, 95%, 96%, 97%, 98%, 99% or 100% identical, or within a range defined by any two of the aforementioned percentages, to a sequence of SEQ ID NOS: 7, 8, 9, 10, 11 or 12.

In some alternatives, the nucleic acid includes a protospacer adjacent motif (PAM) sequence encoded by any one of SEQ ID NOS: 13-18.

Some alternatives include an sgRNA that cleaves DNA directly at a sickle mutation, such as SCL-g1 (also referenced herein as “SCL-1” or “g1”). For example, along with Cas9 nuclease, CRISPR alternatives may include the introduction of an sgRNA containing an approximately 20-base sequence specific to the target DNA 5′ of a non-variable scaffold sequence. A sgRNA may be delivered as RNA or by transforming a cell with a plasmid with the sgRNA-coding sequence under the control of a promoter.

In some alternatives, the nucleic acid includes a deletional repair template or a non-deletional repair template such as a template to be delivered by an AAV. In some alternatives, the template includes one or more of SEQ ID NOS: 9, 10, 11, 12, 13, 14, 15, 16, 17, 18, 19, 20 or 21; or includes sequence that is at least 85%, 86%, 87%, 88%, 89%, 90%, 91%, 92%, 93%, 94%, 95%, 96%, 97%, 98%, 99% or 100% identical, or is within a range defined by any two of the aforementioned percentages, to a sequence of any one of SEQ ID NOS: 9, 10, 11, 12, 13, 14, 15, 16, 17, 18, 19, 20 or 21. In some alternatives, a nucleotide sequence of the repair template is at least 85%, 86%, 87%, 88%, 89%, 90%, 91%, 92%, 93%, 94%, 95%, 96%, 97%, 98%, 99% or 100% identical, or is within a range defined by any two of the aforementioned percentages, to a sequence of any one of SEQ ID NOS: 24, 25, 26, 27, 28, 29, 30, 31, 32, 33, 34, 35 or 36. The template may include regulators and/or enhancers to maximize homology-directed repair (HDR).

In some alternatives, the nucleic acid includes a TALEN such as is encoded in SEQ ID NO: 22 or SEQ ID NO: 23. In some alternatives, a nucleotide sequence of the TALEN is at least 85%, 86%, 87%, 88%, 89%, 90%, 91%, 92%, 93%, 94%, 95%, 96%, 97%, 98%, 99% or 100% identical, or is within a range defined by any two of the aforementioned percentages, to a sequence encoded by or a sequence in accordance with SEQ ID NO: 22 or SEQ ID NO: 23.

In some alternatives, the nucleic acid includes a single-stranded donor oligonucleotides (ssODN). In some embodiments, the ssODN includes one or more of SEQ ID NOS: 19, 20 or 21. In some alternatives, a nucleotide sequence of the ssODN is at least 85%, 86%, 87%, 88%, 89%, 90%, 91%, 92%, 93%, 94%, 95%, 96%, 97%, 98%, 99% or 100% identical, or is within a range defined by any two of the aforementioned percentages, to a sequence encoded by or a sequence in accordance with any one of SEQ ID NOS: 19, 20 or 21.

### Certain Methods of Editing an HBB Gene

Some embodiments of the methods and compositions provide herein include methods for editing an HBB gene in a cell. In some such embodiments, the editing can include HDR. Some embodiments include (i) introducing a polynucleotide encoding a guide RNA (gRNA) into the cell, or introducing a polynucleotide encoding a TALEN into the cell; and (ii) introducing a template polynucleotide into the cell.

In some embodiments, the gRNA comprises a nucleic acid having at least about 85%, 90%, or 95% identity to the nucleotide sequence of any one of SEQ ID NOs:01-06. In some embodiments, the gRNA comprises a nucleic acid having at least about 85%, 90%, or 95% identity to the nucleotide sequence of any one of SEQ ID NOs:07-12. In some embodiments, the gRNA comprises the nucleotide sequence of any one of SEQ ID NOs:01-06. In some embodiments, the gRNA comprises the nucleotide sequence of SEQ ID NO:01. In some embodiments, the gRNA comprises the nucleotide sequence of SEQ ID NO:07.

In some embodiments, introducing a polynucleotide encoding a gRNA into the cell comprises contacting the cell with a ribonucleoprotein (RNP) comprising a CAS9 protein and the polynucleotide encoding the gRNA. In some embodiments, the CAS9 protein and the polynucleotide encoding the gRNA have a ratio between 0.1:1 and 1:10, or between 1:1 and 1:5. In some embodiments, the CAS9 protein and the polynucleotide encoding the gRNA have a ratio of about 1:2.5.

In some embodiments, the template polynucleotide encodes at least a portion of the HBB gene, or complement thereof. In some embodiments, the template polynucleotide encodes at least a portion of a wild-type HBB gene, or complement thereof. In some embodiments, the at least a portion of the HBB gene comprises exon 1 of the HBB gene.

In some embodiments, a viral vector comprises the template polynucleotide. In some embodiments, the vector is an adeno-associated viral (AAV) vector. In some embodiments, the vector is a self-complementary AAV (scAAV) vector. In some embodiments, the template polynucleotide comprises at least about 4 kb of the HBB gene.

In some embodiments, a single-stranded donor oligonucleotide (ssODN) comprises the template polynucleotide.

In some embodiments, the ssODN comprises a nucleotide sequence having at least 80%, 85%, 90%, or 95% identity to the nucleotide sequence of any one of SEQ ID NOs:64-72. In some embodiments, the ssODN comprises a nucleotide sequence any one of SEQ ID NOs:64-72.

In some embodiments, a double-stranded break is created in exon 1 of the HBB gene. In some embodiments, the double-stranded break is created adjacent to the sixth codon in exon 1 of the HBB gene.

In some embodiments, step (i) is performed before step (ii). In some embodiments, steps (i) and (ii) are performed simultaneously. In some embodiments, steps (i) and/or (ii) comprise performing nucleofection. In some embodiments, performing nucleofection comprises use of a LONZA system. In some embodiments, the system comprises use of a square wave pulse. In some embodiments, steps (i) and/or (ii) comprise contacting about 200,000 cells/20 μl nucleofection reaction, wherein the nucleofection reaction comprises the gRNA and/or the template polynucleotide.

In some embodiments, the cell is mammalian. In some embodiments, the cell is human. In some embodiments, the cell is a primary cell. In some embodiments, the cell is a hematopoietic stem cell (HSC). In some embodiments, the cell is a T cell or a B cell. In some embodiments, the cell is a CD34+ cell.

In some embodiments, the HBB gene has at least 95% identity with the nucleotide sequence of SEQ ID NO:37.

### Methods of Therapy

Sickle cell disease (SCD) is caused by a single nucleotide transversion in exon 1 of the HBB gene, resulting in a glutamic acid to valine substitution at the 6th amino acid (E6V). This change increases the hydrophobicity of the adult globin (β^{A}) and renders it susceptible to polymerization resulting in the characteristic sickling pattern of erythrocytes. Sickle patients remain transfusion-dependent with increased morbidity and a reduced life-span. Gene editing with a nuclease in the presence of a donor template (either recombinant adeno-associated virus (rAAV) or ssODN) can fix mutations and drive template-driven repair by the cellular repair machinery. For optimal benefit, clinical gene editing in SCD would lead to efficient donor-directed nucleotide change while concurrently limiting on target HBB nuclease-driven gene disruption via NHEJ.

Accordingly, some alternatives provided herein relate to treating, ameliorating, inhibiting, or improving SCD using a therapeutic genome editing approach. In some alternatives, systems and methods for the introduction of an intact HBB cDNA under control of the endogenous promoter and enhancer in HSPCs is provided. In some alternatives, the systems and methods described herein rescue immunologic and functional defects in HBB and provide a curative therapy.

Some alternatives relate to a method of editing an HBB gene. For example, the method may include providing a cell comprising an HBB gene. In some alternatives, the method includes providing to the cell one or more of the nucleic acid compositions described herein such as a sequence in accordance with, or encoded by, one or more of SEQ ID NOS: 1-36, or, for example, a sequence that is 85%, 86%, 87%, 88%, 89%, 90%, 91%, 92%, 93%, 94%, 95%, 96%, 97%, 98%, 99% or 100% identical, or within a range defined by any two of the aforementioned percentages, to a sequence in accordance with, or encoded by, any one of SEQ ID NOS: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36.

In some alternatives, the cell is a mammalian cell, a human cell, a primary cell, a lymphocyte, a CD34+ stem cell, a hepatocyte, a cardiomyocyte, a neuron, a glial cell, a muscle cell or an intestinal cell, or any of the cells described herein. In some alternatives, a nucleic acid is provided to a cell by transducing the cell with a viral vector or infecting the cell with a virus. In some alternatives, the viral vector is an Adeno-associated virus (AAV) vector such as a recombinant AAV. The AAV may be one or a mixture of multiple serotypes such as serotype 6. For example, a recombinant serotype 6 AAV (rAAV6) is used in some alternatives.

In some alternatives, the nucleic acid is codon-optimized for expression in a host cell, for example in a eukaryotic cell such as a human cell. Some alternatives include providing to the cell a second nucleic acid encoding a gene-editing protein such as a Cas9 protein. In some alternatives, the second nucleic acid is a separate nucleic acid from the first nucleic acid such as a nucleic acid encoding one or more AAV genes, but may be combined with the first nucleic acid. In some alternatives, providing the nucleic acid results in one or more edits to the HBB gene such as, for example, a correction to a sickle cell mutation. In some alternatives, the correction to the sickle cell mutation includes a correction of an E7V mutation. In some alternatives, providing the nucleic acid results in a broken phosphodiester bond in exon 1 of the HBB gene.

Some alternatives, the method further includes engrafting the cell into a subject's bone marrow. In some alternatives, the cell is from a subject, and the cell is from the same subject as the bone marrow. In other words, the cell to be engrafted may be homogeneic with the subject's cells or bone marrow. In some alternatives, the cell is allogeneic to the subject's cells or bone marrow.

### Certain Sequences

Some embodiments include one or more sequences from SEQ ID NOS: 1-36. TABLE 1 includes the sequences of SEQ ID NOS: 1-21, which are relatively short compared to SEQ ID NOS: 22-36 which are further described in TABLE 2. SEQ ID NOS: 1-6 are sgRNA target sequences. SEQ ID NOS: 1 and 6 include antisense strand sequences, and SEQ ID NOS: 2-5 include sense strand sequences. SEQ ID NOS: 1-36. are in a 5′ to 3′ direction. SEQ ID NO: 36 is similar to SEQ ID NO: 35, but does not have a direct repeat of the HBB promoter, and therefore has a small deletion. This rAAV6 template (SEQ ID NO: 36) drives HBG1 promoter driving a E7V mutation into the HBB locus and retains the native intron 1. MND-GFP is in the reverse orientation to prevent promoter interference in SEQ ID NO: 36. TABLE 2 provides additional information with regard to SEQ ID NOS: 24-36. SEQ ID NO: 22 is a DNA molecule with a sequence length of 15,662 and size of 49 KB, while SEQ ID NO: 23 is a DNA molecule with a sequence length of 15,866 and size of 54 KB. The vector for SEQ ID NOS: 22 and 23 is: pEVL300 noBsmBI GG compatible from pWNY2.0.

TABLE 1

| SEQ ID | | |
|---|---|---|
| NO: | Identifier(s) | Sequence |
| 1 | g1 or SCL-1 | GTAACGGCAGACTTCTCCTC |
| 2 | g2 or SCL-2 | GTCTGCCGTTACTGCCCTGT |
| 3 | g3 or SCL-3 | TCTGCCGTTACTGCCCTGT |
| 4 | g4 or SCL-4 | AGTCTGCCGTTACTGCCCTG |
| 5 | g5 or SCL-5 | AAGGTGAACGTGGATGAAGT |
| 6 | g6 or SCL-6 | CTTGCCCCACAGGGCAGTAA |
| 7 | SCL-1 + gRNA scaffold | GTAACGGCAGACTTCTCCTCGTTTTAGAGCTAGAAAT |
| | | AGCAAGTTAAAATAAGGCTAGTCCGTTATCAACTTG |
| | | AAAAAGTGGCACCGAGTCGGTGCTTTT |
| 8 | SCL-2 + gRNA | GTCTGCCGTTACTGCCCTGTGTTTTAGAGCTAGAAAT |
| | scaffold | AGCAAGTTAAAATAAGGCTAGTCCGTTATCAACTTG |
| | | AAAAAGTGGCACCGAGTCGGTGCTTTT |
| 9 | SCL-3 + gRNA | TCTGCCGTTACTGCCCTGTGTTTTAGAGCTAGAAATA |
| | scaffold | GCAAGTTAAAATAAGGCTAGTCCGTTATCAACTTGA |
| | | AAAAGTGGCACCGAGTCGGTGCTTTT |
| 10 | SCL-4 + gRNA | AGTCTGCCGTTACTGCCCTGGTTTTAGAGCTAGAAAT |
| | scaffold | AGCAAGTTAAAATAAGGCTAGTCCGTTATCAACTTG |
| | | AAAAAGTGGCACCGAGTCGGTGCTTTT |
| 11 | SCL-5 + gRNA | AAGGTGAACGTGGATGAAGTGTTTTAGAGCTAGAAA |
| | scaffold | TAGCAAGTTAAAATAAGGCTAGTCCGTTATCAACTT |
| | | GAAAAAGTGGCACCGAGTCGGTGCTTTT |
| 12 | SCL-6 + gRNA | CTTGCCCCACAGGGCAGTAAGTTTTAGAGCTAGAAA |
| | scaffold | TAGCAAGTTAAAATAAGGCTAGTCCGTTATCAACTT |
| | | GAAAAAGTGGCACCGAGTCGGTGCTTTT |
| 13 | SCL-1 PAM | AGG |
| | sequence | |
| 14 | SCL-2 PAM | GGG |
| | sequence | |
| 15 | SCL-3 PAM | GGG |
| | sequence | |
| 16 | SCL-4 PAM | TGG |
| | sequence | |
| 17 | SCL-5 PAM | TGG |
| | sequence | |
| 18 | SCL-6 PAM sequence | CGG |
| 19 | ssODN-V7E: | TCAGGGCAGAGCCATCTATTGCTTACATTTGCTTCTG |
| | GTC | ACACAACTGTGTTCACTAGCAACCTCAAACAGACAC |
| | | CATGGTGCATCTGACTCCTGTCGAGAAGTCTGCCGTT |
| | | ACTGCCCTGTGGGGCAAGGTGAACGTGGATGAAGTT |
| | | GGTGGTGAGGCCCTGGGCAGGT |
| 20 | ssODN-V7E: | TCAGGGCAGAGCCATCTATTGCTTACATTTGCTTCTG |
| | GTG | ACACAACTGTGTTCACTAGCAACCTCAAACAGACAC |
| | | CATGGTGCATCTGACTCCTGTGGAGAAGTCTGCCGTT |
| | | ACTGCCCTGTGGGGCAAGGTGAACGTGGATGAAGTT |
| | | GGTGGTGAGGCCCTGGGCAGGT |
| 21 | ssODN | TCAGGGCAGAGCCATCTATTGCTTACATTTGCTTCTG |
| | CCCGAA | ACACAACTGTGTTCACTAGCAACCTCAAACAGACAC |
| | | CATGGTGCATCTGACTCCCGAAGAGAAGTCTGCCGT |
| | | TACTGCCCTGTGGGGCAAGGTGAACGTGGATGAAGT |
| | | TGGTGGTGAGGCCCTGGGCAGGT |

TABLE 2

| SEQ | | | |
|---|---|---|---|
| ID | | Sequence | |
| NO: | Alternative name or descriptor | length | Size |
| 22 | FrankenpEVL_SCL L Talen | | |
| 23 | FrankenpEVL_SCL R Talen | | |
| 24 | AMS#1242 pAAV | 7365 | 40 KB |
| | HBB(400)>synTron.HBBopt(T87Q). | | |
| | wPRE-O.BGHpA;MND>GFP::P2A::Ex2 | | |
| 25 | AMS#1243 pAAV | 8087 | 42 KB |
| | HBB(400)>synTron.HBBopt(T87Q).wPRE- | | |
| | O.BGHpA;HPFH2.MND>GFP::P2A::Ex2 | | |
| 26 | AMS#1244 pAAV | 7712 | 49 KB |
| | HBB(400)>synTron.HBBopt(T87Q).wPRE3. | | |
| | USE-SV40pA;HPFH2.MND>GFP::P2A::Ex2 | | |
| 27 | AMS#1245 pAAV | 7925 | 57 KB |
| | HBB(400)>HBBopt(T87Q).wPRE- | | |
| | O.BGHpA.HPFH2;MND>GFP::P2A::Ex2 | | |
| 28 | AMS#1246 pAAV HBB(600)>HBBopt(E7V) | 5216 | 31 KB |
| | Sickle un-repair | | |
| 29 | AMS#1247 pAAV HBB(400)>HBBopt(E7V) | 4816 | 23 KB |
| | Sickle un-repair | | |
| 30 | AMS#1248 pAAV HBB(200)>HBBopt(E7V) | 4416 | 28 KB |
| | Sickle un-repair | | |
| 31 | AMS#1249 pAAV HBB(50)>HBBopt(E7V) | 4116 | 22 KB |
| | Sickle un-repair | | |
| 32 | AMS#1289 pAAV | 7995 | 78 KB |
| | HBB(400)d0>HBBopt(T87Q).wPRE- | | |
| | O.BGHpA.HPFH2;MND>GFP.SV40pA | | |
| 33 | AMS#1290 pAAV | 7995 | 79 KB |
| | HBB(400)d14>HBBopt(T87Q).wPRE- | | |
| | O.BGHpA.HPFH2;MND>GFP.SV40pA | | |
| 34 | AMS#1314 pAAV HBB(2.1kb).SCmutation | 8036 | 64 KB |
| 35 | AMS#1321 pAAV HBB(800).d53,71 | 8207 | 95 KB |
| | [MND>GFP.SV40pA]; | | |
| | HBG1d13p>HBB(E7V)opt | | |
| 36 | AMS#1322 pAAV HBB(7,800).d-127,71 | 8009 | 90 KB |
| | [MND>GFP.SV40pA]; | | |
| | HBG1d13p>HBB(E7V)opt | | |

### EXAMPLES

### Example 1—Comparison of rAAV6 and ssODN for HDR at the β-Globin (HBB) Locus

The impact and clinical relevance of rAAV6 and ssODN delivery in correcting SCD was evaluated by introducing a E6V sickle mutation into human mobilized peripheral blood CD34+ cells (hPBSCs) using a Crispr/Cas9 ribonucleoprotein (RNP). Two donor delivery strategies were employed: a rAAV6 (AMS #1314) with 2.2 kb homology arms (HA) and a ssODN comprising 168 nucleotides (E7V-GTC and E7V-GTG change and V7E with CCCGAA change). The efficiency of HDR in comparison to residual NHEJ rates was evaluated following Crispr/Cas9 RNP generated double-stranded breaks (3% rAAV6, AMS #1314 and 12.5, 25, 50, or 100 pmol of ssODN).

Human mobilized peripheral blood CD34+ cells were thawed for 48 hours in SCGM media containing cytokines: 100 ng/ml of SCF, IL-6, Flt-3L, TPO. Cells were electroporated at 48 hours post-thaw and added to virus-containing recovery media (3% rAAV6, AMS #1314) for rAAV6 delivery. A dose titration of ssODN containing 12.5, 25, 50, or 100 pmol of E7V ssODN with a GTC or GTG change or V7E with a CCCGAA change was electroporated along with RNP and then added to recovery media. After 18-24 hours, cells were transferred to differentiation media containing IMDM with 1% Pen/Strep, 20 ng/mL hSCF, 1 ng/mL hIL-3, 2 IU/mL EPO and 20% heat-inactivated FBS. Cells were differentiated for 14 days and the erythroid cells were analyzed for various globin sub-types.

Upon in vitro testing, the rates of HDR:NHEJ on day 10 post-editing was 31%:18% across 5 CD34+ donors tested using rAAV6 (AMS #1314) and 13%:30% across 3 CD34+ donors tested using ssODN E7V GTG (˜ratio of 2:1 vs. 1:2, respectively). The edited CD34+ cells were differentiated for 2 weeks into erythroid cells and the amount of β^{S} (sickle globin) in the erythroid precursors were quantified using RP-HPLC. The amount of β^{S} was 18-27% (n=5) with rAAV6 template delivery and 0-9% (n=3) using ssODN delivery, respectively. Thus, rAAV6 or ssODN E7V GTG successfully introduced a targeted nucleotide change within the HBB locus. rAAV6(AMS #1314) was the superior method for introducing a targeted nucleotide change within the HBB locus. These findings also highlight a benefit of measuring both HDR:NHEJ ratios and therapeutic protein levels as metrics in assessing the potential for clinical gene editing designed to introduce a nucleotide change while limiting on-target gene disruption.

### Example 2—HDR Template Design and Delivery to Edit and Correct the Sickle Mutation within the Exon-1 of the HBB Gene

To edit and correct a sickle mutation, nucleases that edit at the E7V mutation of the HBB gene were developed. Both TALENs as well as Crispr/Cas9 ribonucleoprotein-mediated delivery of chemically modified single guide RNA (sgRNA) were optimized to edit at exon 1 of the HBB gene. The data showed efficient editing at the sickle locus in K562 and human hematopoietic stem cells (CD34+). Various repair template architecture were designed for rAAV6-mediated delivery of novel HDR repair templates with unique regulatory elements. Anti-sickling (T87Q) globin cassettes, sickle globin introduction and sickle correction cassettes were tested at the HBB locus. The design of these templates was unique. Efficient, clinically-relevant rates of homology-dependent repair (HDR) at the HBB locus were achieved.

ssODN delivery of repair templates were also designed and optimized to drive HDR at the HBB locus. ssODNs that introduce the sickle mutation as well as correct the sickle mutation were designed and tested. Both mono- or bi-allelic HDR integration was achieved at the sickle locus. Further, clinically-relevant globin expression was observed from the integrated templates, irrespective of the mode of delivery.

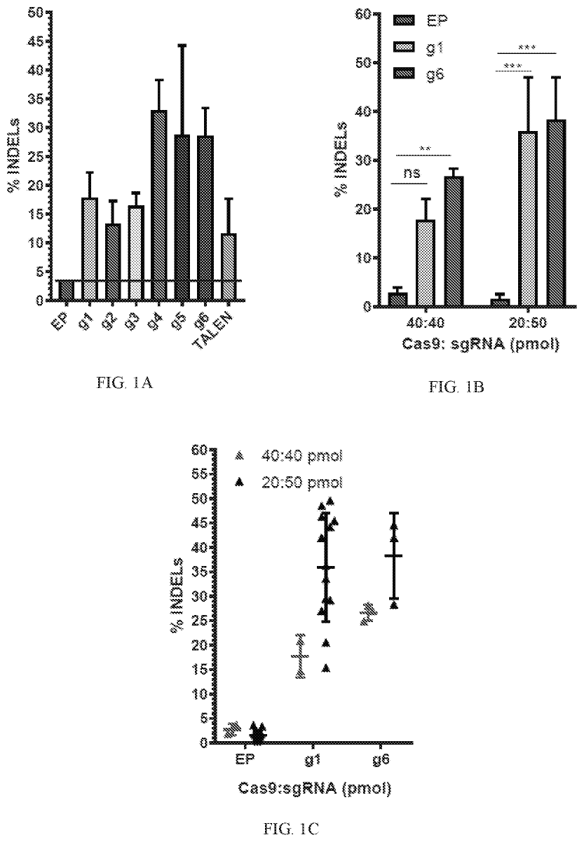

RNPs and TALENs were screened to create double strand (ds) breaks in Exon 1 of the human HBB gene. FIG. 1A shows nuclease efficiency comparing various sgRNAs delivered as RNPs and TALENs. The nucleases that resulted in the highest % INDELs were g4, g5 and g6. Cas9:sgRNA ratios were configured to maximize editing efficiency, and a ratio of 20:50 Cas9:sgRNA resulted in a higher % INDELs than a ratio of 40:40 (FIG. 1B). As shown in FIG. 1C, editing efficiencies were evaluated across different CD34+ donors using sgRNA 1 and 6 delivered as RNP. The results show that efficient editing at the HBB locus was achieved with nucleases.

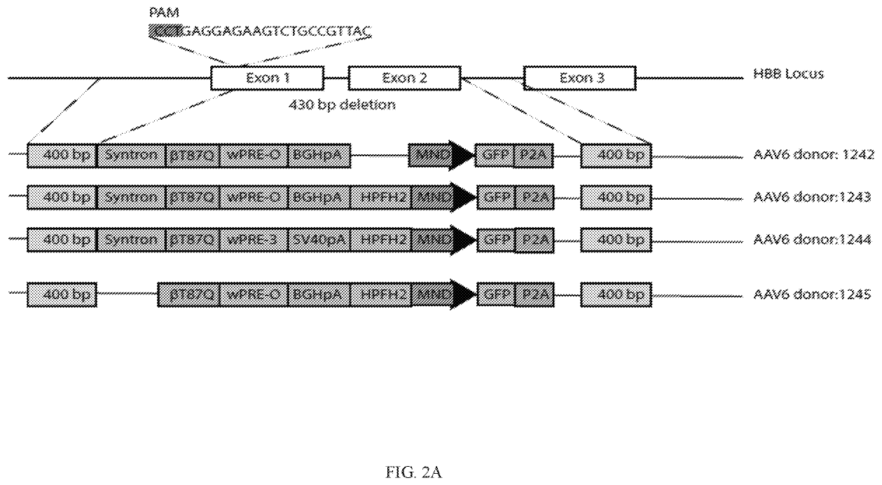

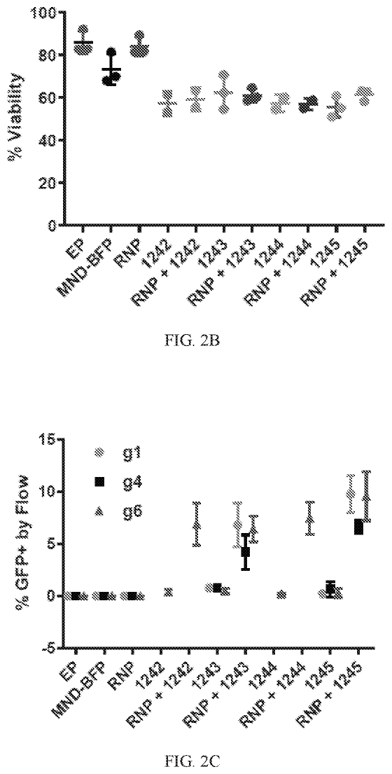

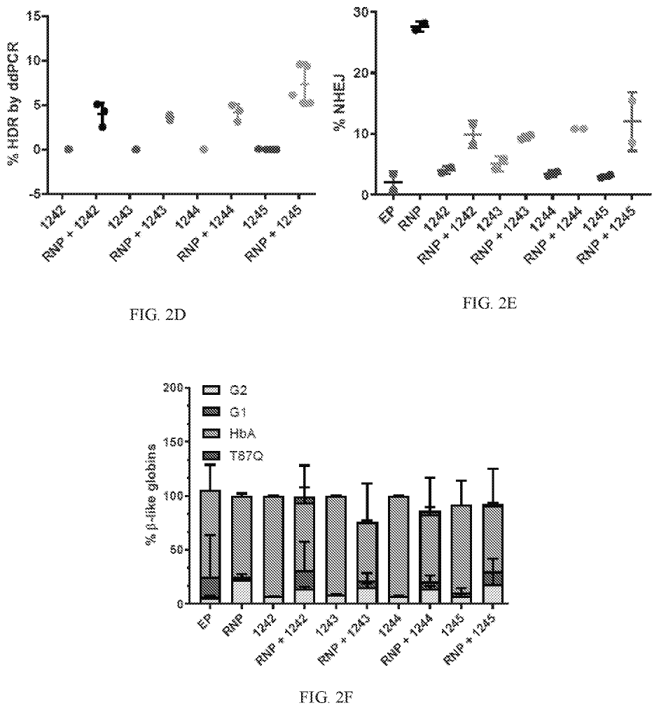

Regulators and enhancers were configured to maximize HDR using deletional templates 1242-1245 introducing an anti-sickling globin (β^{T87Q}). FIG. 2A includes a schematic of deletional templates 1242-1245, and shows elements used in each. As shown in FIG. 2B, viability of CD34+ cells was determined on day 2 post-electroporation and AAV6 transduction. Transduction with the deletional repair templates tended to result in about 60% viability. FIG. 2C shows a comparison of HDR with 3 different guides delivered as RNP with deletional templates 1242-1245. As shown in FIG. 2D, the relative HDR % based on ddPCR using RNP and deletional templates was determined with deletional templates 1242-1245. Editing efficiencies were also determined using TIDE sequencing analysis (FIG. 2E) and HPLC analysis (FIG. 2F) looking at β-globin expression from cells differentiated for 2 weeks in erythroid differentiation media. The results indicate that deletional repair templates with an anti-sickling globin were effectively and efficiently delivered to cells by rAAV6, and integrated into the cells' genomes.

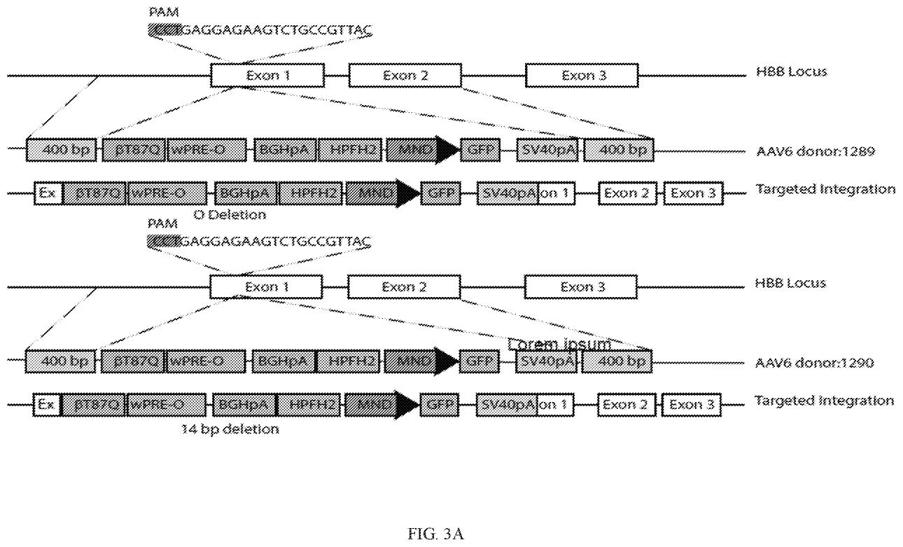

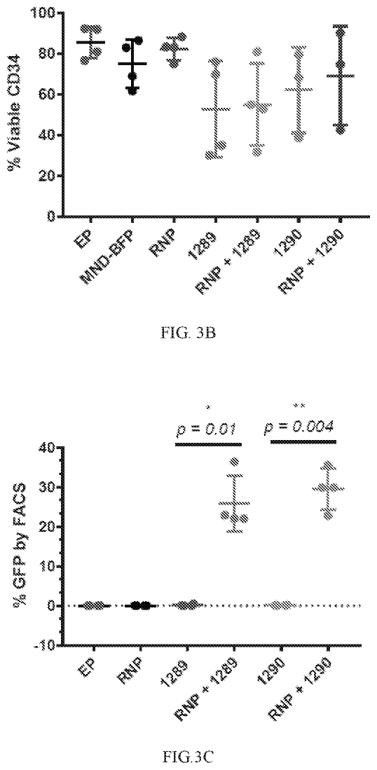

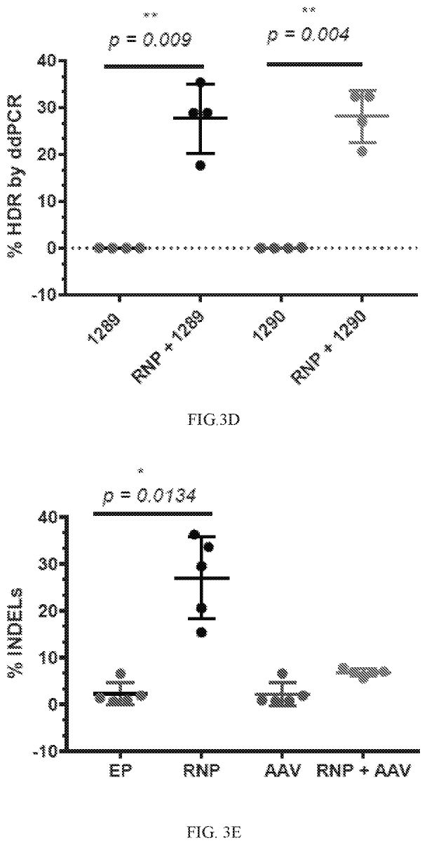

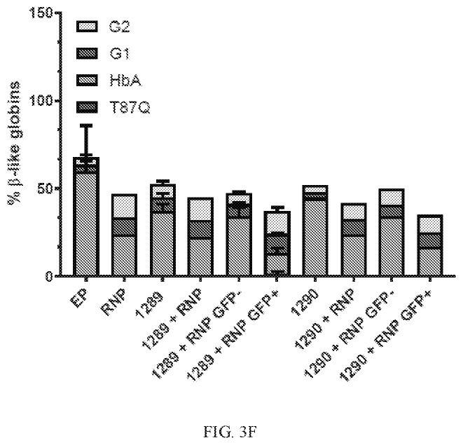

HDR with non-deletional templates were also configured to introduce the anti-sickling globin β^{T87Q}. FIG. 3A includes a schematic of non-deletional templates 1289-1290, and shows elements used in each. As shown in FIG. 3B, viability of CD34+ cells on day 2 post-electroporation and AAV6 transduction was determined, and also tended to be at about 60%, and ranged from about 30% to about 90%. FIG. 3C shows HDR % based on flow cytometry using RNP and non-deletional templates; 1289-1290. As shown in FIG. 3D, the relative HDR % based on ddPCR using RNP and non-deletional templates was determined for templates 1289 and 1290. Editing efficiencies were also determined using TIDE sequencing analysis (FIG. 3E) and HPLC analysis (FIG. 3F) looking at β-globin expression from cells differentiated for 2 weeks in erythroid differentiation media in bulk GFP+ population and GFP+/− sorted cells. The results indicated that non-deletional repair templates with an anti-sickling globin were effectively and efficiently delivered to cells by rAAV6, and changes to the HBB locus were effectively made.

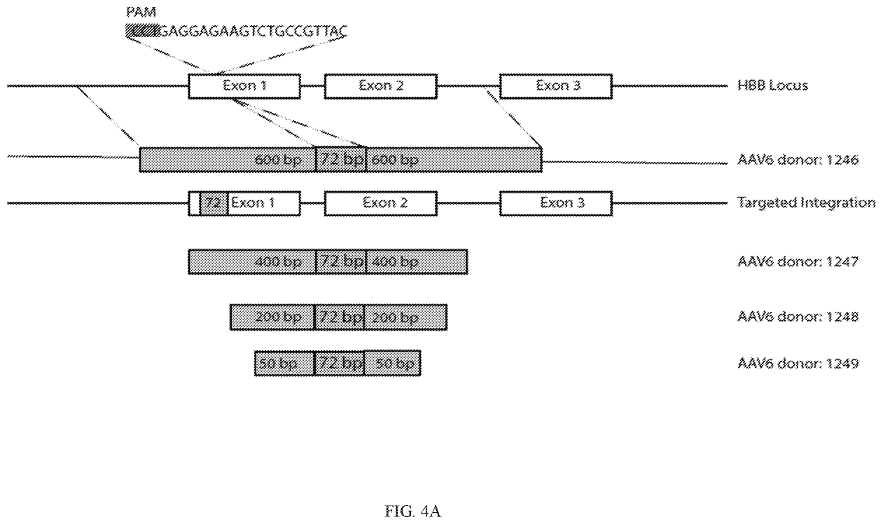

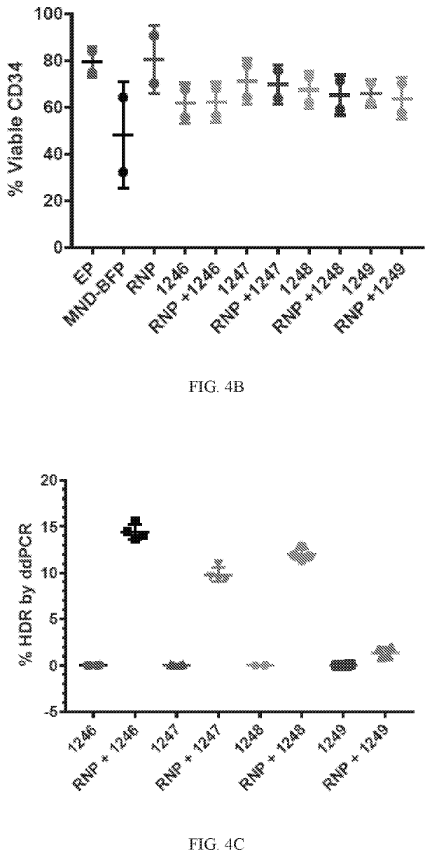

HDR with human codon-optimized templates with varying HR arm lengths introducing a sickle mutation were developed. FIG. 4A includes a schematic of human codon-optimized templates 1246-1249. As shown in FIG. 4B, viability of CD34+ cells on day 2 post-electroporation and AAV6 transduction was determined. FIG. 4C shows absolute HDR % based on ddPCR using RNP and human codon-optimized templates 1246-1249. As shown in FIG. 4D, an HPLC analysis was performed looking at β-globin expression from cells differentiated for 2 weeks in erythroid differentiation media. The results indicate that human codon-optimized templates with an anti-sickling globin were effectively and efficiently delivered to cells by rAAV6, and changes to the HBB locus were effectively made.

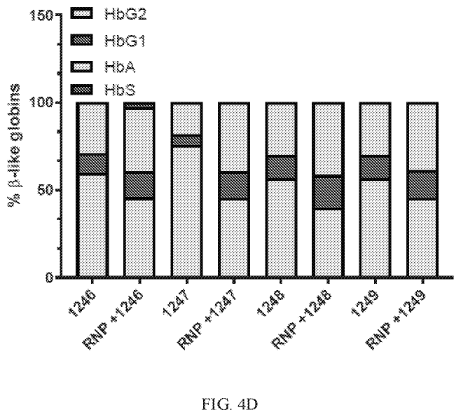

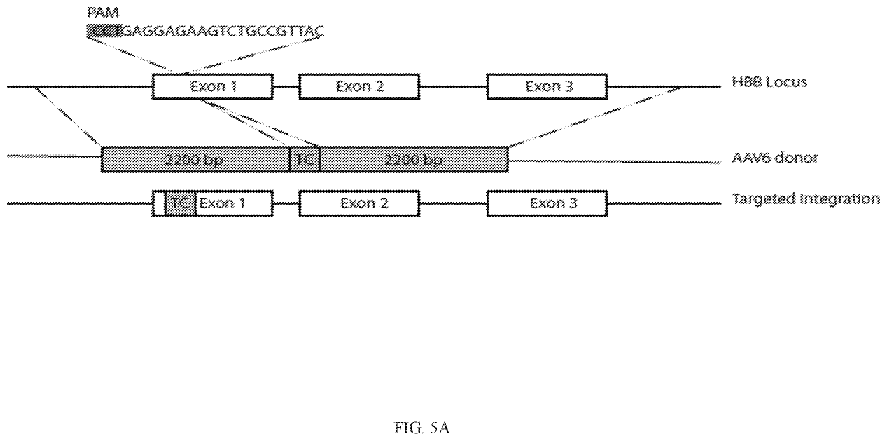

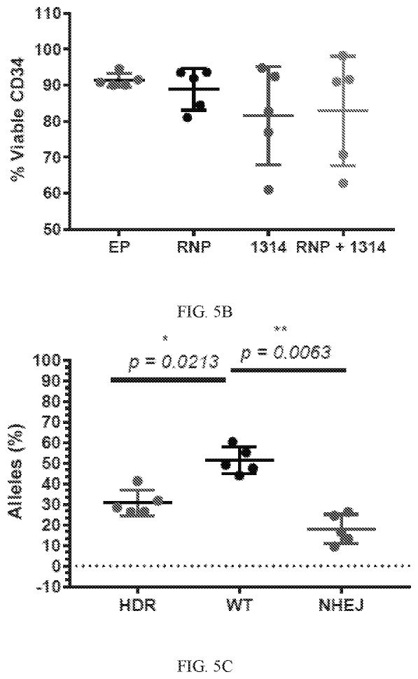

HDR with template 1314 was effective for introducing a sickle mutation. FIG. 5A includes a schematic of template 1314. As shown in FIG. 5B, viability of CD34+ cells was determined on day 2 post-electroporation and AAV6 transduction. FIG. 5C shows results of colony sequencing of samples edited with RNP and template 1314 from 5 different donors. Editing efficiencies were determined using TIDE sequencing (FIG. 5D) and HPLC analysis (FIG. 5E) of 4 donors looking at β-globin expression from cells differentiated for 2 weeks in erythroid differentiation media. A chromatogram (FIG. 5F) was produced that shows various globin subtypes from a day 14 differentiated HDR sample. The results indicate that sickle mutation introduction was achieved after delivery of a template delivered by rAAV6.

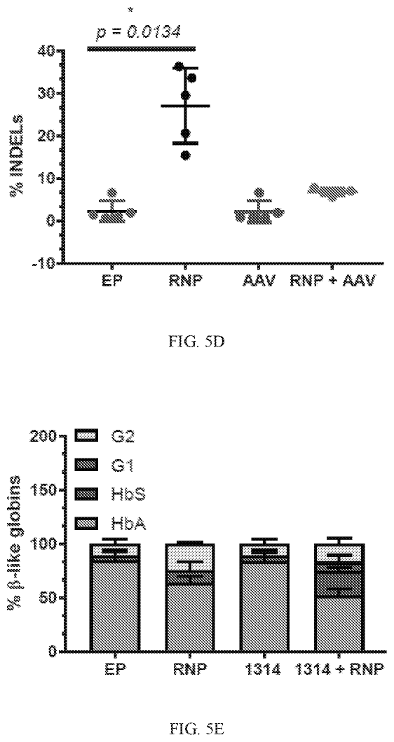

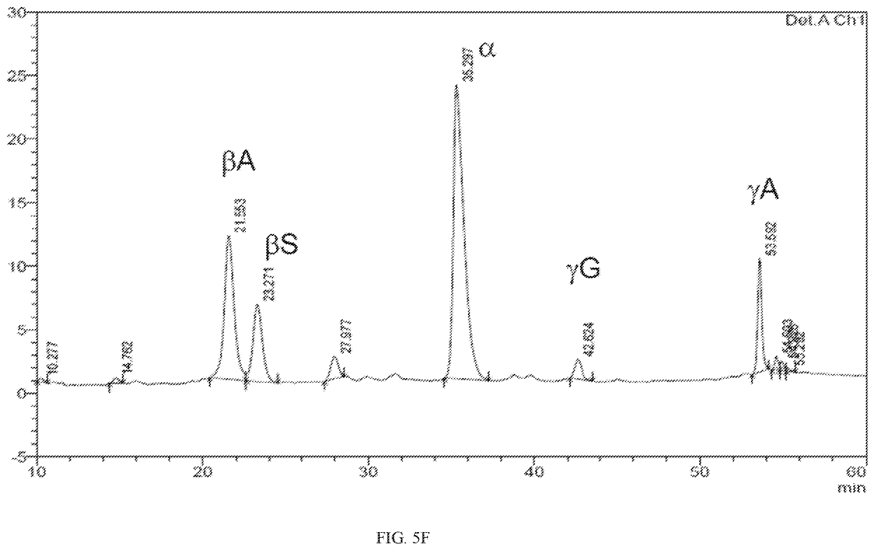

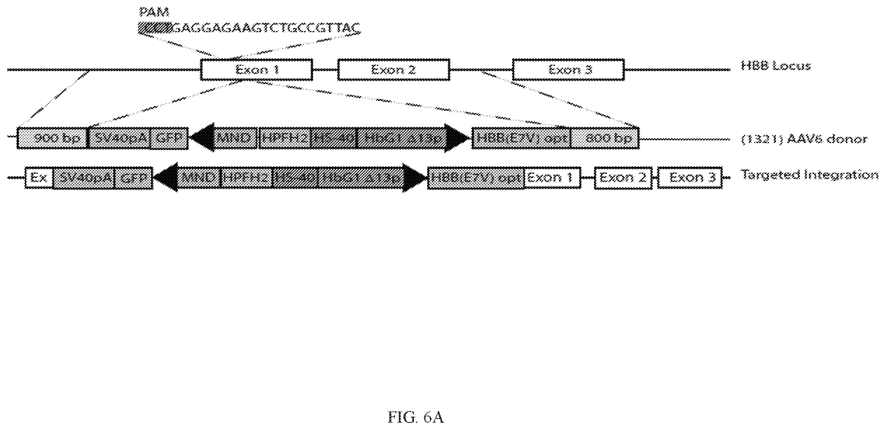

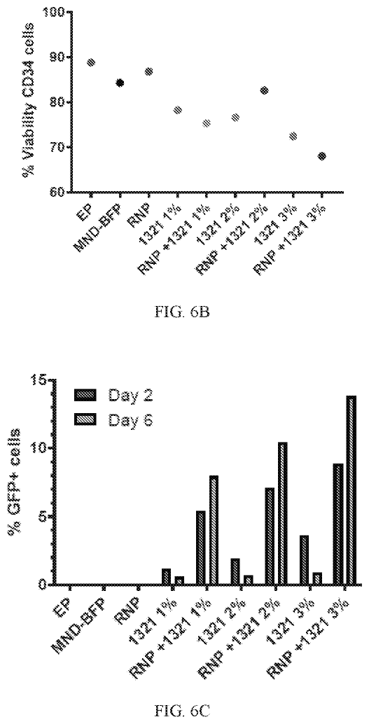

HDR with a non-deletional template was configured for introducing the sickle mutation. FIG. 6A includes a schematic of the non-deletional template, 1321. As shown in FIG. 6B, viability of CD34+ cells was determined on day 2 postelectroporation and AAV6 transduction. HDR events were measured in the edited samples with template 1321 (FIG. 6C). The results indicate that sickle mutation introduction was achieved after delivery of a non-deletional template delivered by rAAV6.

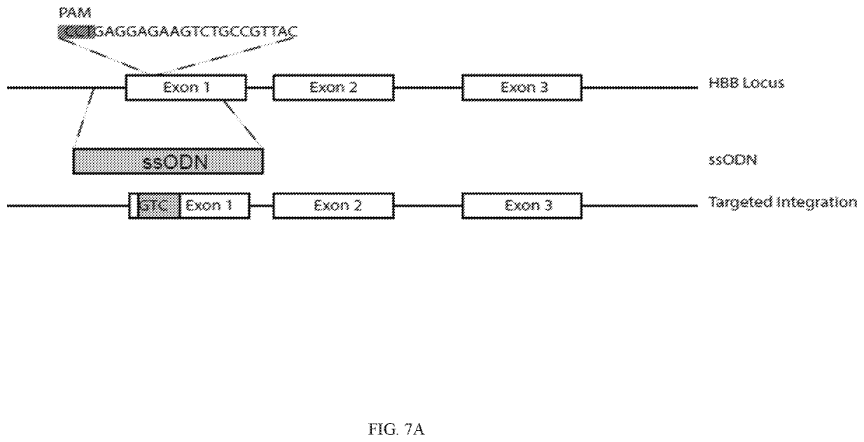

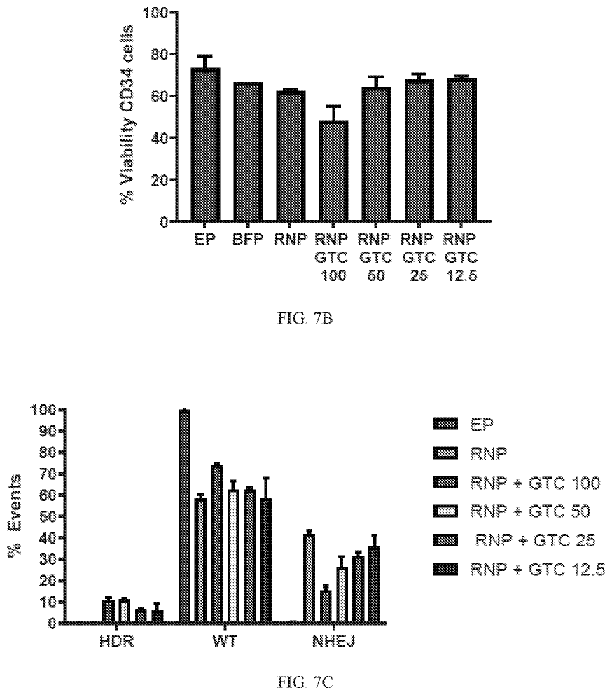

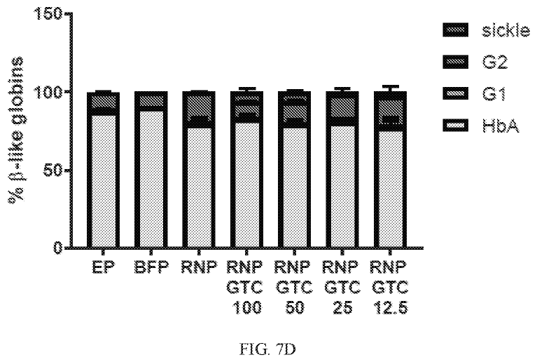

HDR with ssODN was configured for introducing a sickle mutation. FIG. 7A includes a schematic of ssODN E7V. As shown in FIG. 7B, viability of CD34+ cells on day 2 post-electroporation with 100, 50, 25, or 12.5 pmol GTC was determined. These same doses were also used in FIGS. 7C and 7D. HDR and NHEJ were measured by ddPCR in edited samples with a dose titration of E7V ssODN (FIG. 7C). As shown in FIG. 7D, an HPLC analysis of various globin sub-types expressed in erythroid cell was performed. The results indicated that a sickle mutation was achieved after delivery of an ssODN.

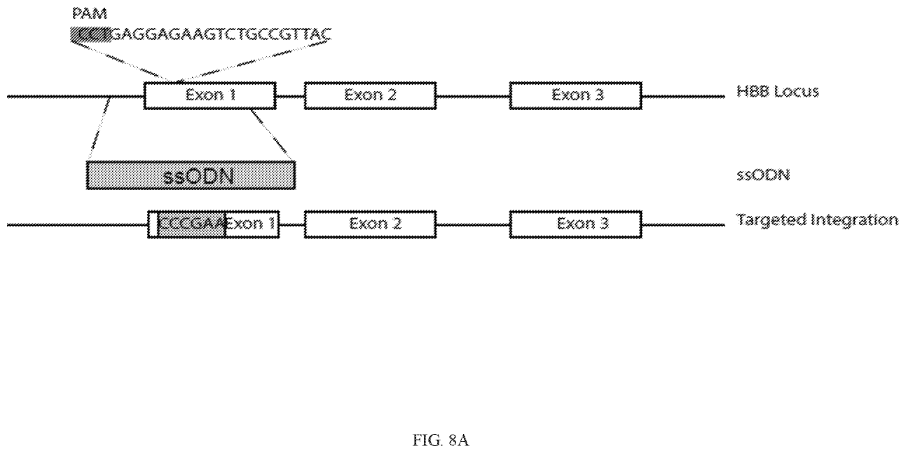

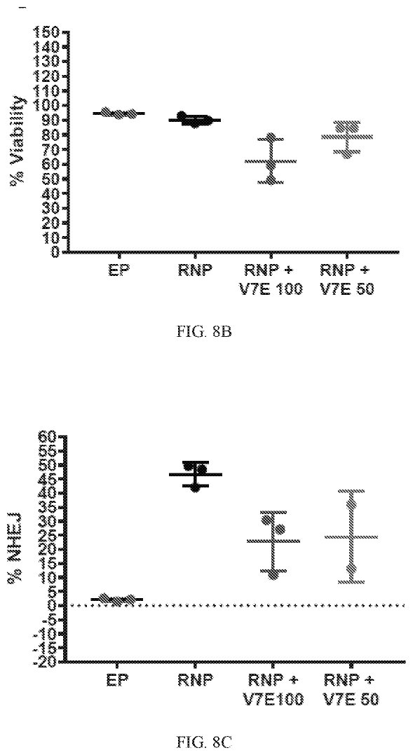

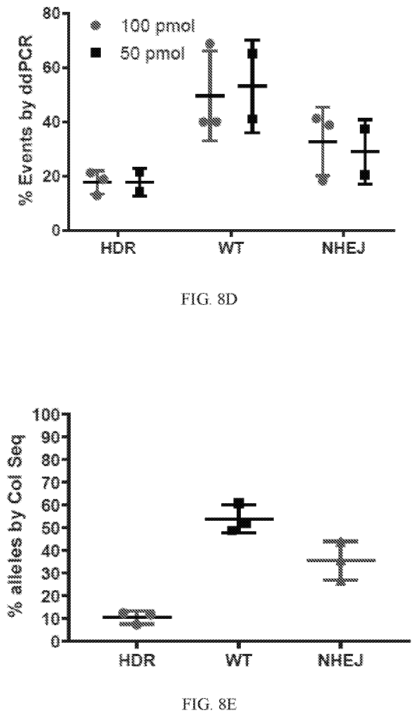

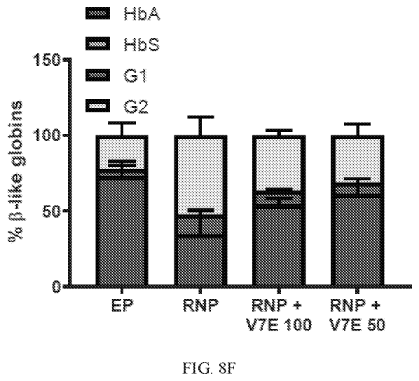

HDR with ssODN was configured for correcting the sickle mutation (CCC GAA). FIG. 8A includes a schematic of ssODN V7E. As shown in FIG. 8B, viability of CD34+ cells was measured on day 2 post-electroporation. INDELS were evaluated by TIDE sequencing of 3 different donors after editing (FIG. 8C). As shown in FIG. 8D, HDR and NHEJ were measured by ddPCR in edited samples with a dose titration of V7E ssODN. As shown in FIG. 8E, HDR and NHEJ were also measured by colony sequencing in edited sample with V7E ssODN. An HPLC analysis of various globin sub-types in erythroid cells was performed (FIG. 8F). The results indicated that an ssODN was effective for delivery and sickle correction.

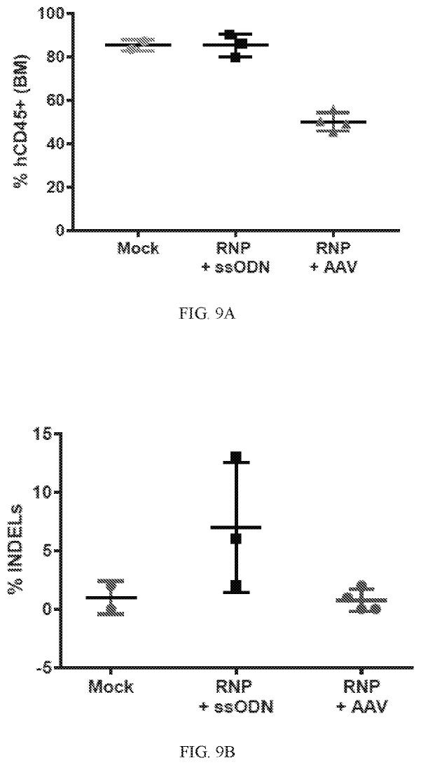

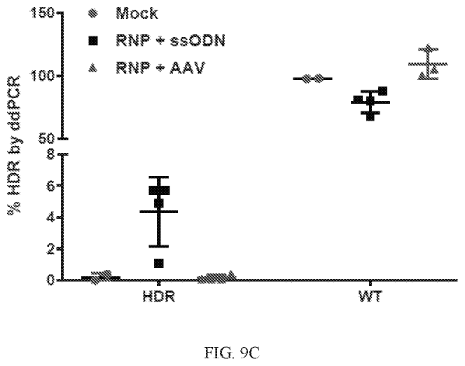

Edited cells were engrafted in W41 SCID mice. FIG. 9A shows the results of human CD45+ engraftment at 12 weeks in bone marrow. The methods for engraftment included treating 6-8-week-old W41 mice with 25 mg/kg of busulfan. Two×10⁶ human cells were delivered by tail-vein injection. The mice were monitored for 12 weeks and overall human chimerism, multi-lineage engraftment and erythroid re-constitution were measured at the time they were sacrificed. The % HDR was measured by ddPCR with human-specific primers and % Indels were measured by TIDE sequencing.

As shown in FIG. 9B, INDELs were measured by TIDE sequencing in the engrafted human cells. As shown in FIG. 9C, HDR was measured by ddPCR in the bone marrow at 12 weeks. The results indicated that edited cells were engrafted into a subject's bone marrow, to produce non-sickle blood cells.

As described herein, various strategies were used to design HDR templates that can be inserted into the HBB locus to correct the sickle mutation. The templates included three groups: Group 1: rAAV6-based HDR templates that have various enhancers, introns, promoters, polyA tails, various homology arm lengths, and/or deletional and non-deletional cassettes that insert T87Q anti-sickling globin into HBB gene. Such templates provided evidence of HDR and showed unique anti-sickling (T87Q) globin expression driven by the insertion of repair template into human cells. Group 2: rAAV6-based HDR templates that have various enhancers, introns, promoters, polyA tails, various homology arm lengths, and/or deletional and non-deletional cassettes that insert sickle mutation or correct the sickle mutation at the HBB gene. Such templates provided evidence of HDR and showed unique sickle globin expression (HbS) or adult hemoglobin (HbA) driven by the insertion of repair template into human cells. Group 3: ssODN-based templates that drive insertion of the sickle mutation or drive sickle correction into the HBB gene. The data provided evidence of HDR and showed unique sickle globin expression or adult hemoglobin (HbA) driven by the insertion of ssODN into human cells.

Examples of novel templates that were designed and tested herein include the following: the rAAV6 HDR repair template design described herein; HDR repair templates delivered as rAAV6 that include 1242, 1243, 1244, 1245, 1246, 1247, 1248, 1249, 1289, 1290, 1314, 1321, 1322; the ssODN design introducing a sickle mutation that drives a GTC change as well as GTG change in codon 7 of exon1 of HBB gene (both E7V cassettes with GTC and GTG change are unique and have not been reported before); and the ssODN design that corrects the sickle mutation and drives a CCC GAA change in codon 6 and 7 of HBB gene.

An sgRNA (SCL-g6) that is 17 bases away from the sickle mutation has been utilized. The HDR templates are delivered as scAAV6 and deliver a long cDNA cassette that integrates at the HBB gene and introduces an anti-sickling HBB cDNA (HbAS3). The cDNA is inserted into the gene start and preserves endogenous promoter/enhancer function. Alternatively the HDR template that has been tested previously uses a rAAV6 E6V donor that has 2.2 kb HR arms with 5 codon-optimized nucleotide sequence change in various codons along with the GTC change at codon 7 (gTCgagaagtctgcAgtCactgcTctAtggggGaaA; SEQ ID NO:38). These templates have been designed to work with SCL guide 6 delivered as a RNP.

ssODN templates that have been attempted previously introduce a E7V GTA (Dewitt et al.) or a V7E GAA change at codon 7 within exon 1 of the HBB gene, and work with SCL-g6 sgRNA that is 17 bases from the sickle mutation. On the other hand, the rAAV6 donor cassettes described herein utilize a novel guide, SCL-g1 that specifically cuts at the sickle mutation. The novel rAAV6 and ssODN repair templates described herein create a non-deletional HDR event that drives unexpectedly high levels of HDR, which is clinically relevant. In some alternatives, these novel donor templates (a) insert an antisickling T87Q globin, (b) introduce a sickle mutation, or (c) deliver a human codon-optimized sickle correction. The novel rAAV6 cassettes described herein utilize unique combinations of promoters, enhancers, polyA tails and regulatory elements to maximize globin expression.

A benefit to the approach described in some embodiments is that editing at the site of the mutation can improve functional outcomes. In some embodiments, the sgRNA cuts at the sickle mutation. In some embodiments, the proximity to the cut site to the homology arms, allows for an improvement in the conversion of the mutation. In some embodiments, the rAAV6 templates or the ssODN templates are specifically created to work with SCL-g1, which edits at the sickle mutation, so as to allow for highly efficient correction of the sickle mutation.

In some embodiments, template design maximizes HDR at the HBB locus. In some embodiments, the HBB locus also has an unexpected propensity for bi-allelic integration, shown herein, that, in some embodiments, provides a major therapeutic benefit.

In some embodiments, globin expression is maximized through selection of regulatory elements. For example, selecting certain regulatory elements can improve or increase expression of T87Q anti-sickling globin or adult globin. In some embodiments, one or more of the following elements modulates driving higher level of HDR and increases globin expression: SV40 polyA tail, HPFH-2 enhancer, and/or a wPRE-3 element

In some embodiments, delivering a human codon optimized sickle correction cassette helps restore functional HbA hemoglobin in sickle patients. In some embodiments, having the native intron 1 in a proximal location allows for maximum globin expression.

### Example 3—In Vivo Outcome of Homology-Directed Repair at the HBB Gene in HSC Using Alternative Donor Template Delivery Methods

### Experimental Protocols

rAAV6 production: rAAV6 stocks were produced. The rAAV6 vector, serotype helper and HgT1-adeno helper plasmids were transfected into HEK293T cells. Cells were harvested at 48 hours, lysed and treated with benzonase. An iodixanol density gradient was used to purify the virions with recombinant rAAV6 genomes. The qPCR-based titers of rAAV6 genomes were determined by using ITR specific primers and probe. 1%, 2% and 3% of the culture volume were used for transducing rAAV6 into mPBSCs.

CD34⁺ hematopoietic stem cells: frozen mPBSC were purchased from Cooperative Center for Excellence in Hematology at Fred Hutchinson Cancer Research Institute, Seattle, Wash.

sgRNA and TALEN design: Guides were designed that were predicted to cut close to the sickle mutation using CRISPR design tools, (http://crispr.mit.edu/ and http://crispor.tefor.net/). All guides were synthesized as chemically modified 2′-O-methyl analogs with 3′ phosphorothioate inter-nucleotide linkages in the first three 5′ and 3′ terminal residues (Synthego Inc., CA). TALENs that cut at the sickle mutation were assembled with a Golden Gate cloning strategy. TALEN mRNA was produced based on previously published protocols (Grier, A E, et al., (2016). Mol Ther Nucleic Acids 5: e306; hereby expressly incorporated by reference in its entirety).

Electroporation, transduction of cells and erythroid differentiation culture: Alt-R S.p Cas9 Nuclease 3NLS protein was used for all studies (Integrated DNA Technologies Inc., Coralville, Iowa). The CD34⁺ cells were cultured in SCGM media (CellGenix, New Hampshire) with 100 ng/ml each of FLT-3 ligand, TPO, hSCF and IL-6 (Peprotech, Rocky Hill, N.J.). Cells were electroporated 48 hours after thaw using NEON electroporation system (ThermoFischer Scientific, Waltham, Mass.) at 1300 V, 20 millisec and 1 pulse or the Lonza 4-D nucleofector (Lonza, Basel, Switzerland, CM149 protocol). The Cas9 RNP was made right before electroporation or nucleofection by mixing 20 pmol of Cas9 and 50 pmol of sgRNA (per 2×10⁵ cells, ratio of 1:2.5 of Cas9: sgRNA). The RNP mixture was made fresh and incubated at room temperature for 15 minutes. ssODN donor templates were used at 100, 50, 25, 12.5 pmol for every 2×10⁵ cells and was added into the mixture of RNP right before electroporation or nucleofection. Cells after electroporation or nucleofection were added to either rAAV6 containing SCGM media with cytokines (at a 1%, 2% or 3% culture volume; 3% GTC rAAV6˜MOI of 4500-5100, 1% GAA rAAV6˜MOI of 2190) or to plain SCGM media with cytokines for ssODN treated and control cells. The cells were incubated in media overnight at 37° C. for 18 hours. After 18 hours the cells were transferred to tissue culture non-treated plates containing IMDM media with 1 ng/ml hIL-3, 2 IU/ml EPO, 20 ng/ml h-SCF, 20% heat-inactivated FBS and 1% pen/strep. (Fisher Scientific, Hampton, N H and Peprotech, Rocky Hill, N.J.). The cell density was kept between 5×10⁵ to 1×10⁶ cells/ml to minimize fetal hemoglobin induction due to proliferative stress or over-crowding. CD235 expression was monitored at day 14 by flow cytometry using BV421-labelled Glycophorin-A antibody (BD, 562938).

Measuring HDR events with rAAV6 and ssODN using ddPCR: gDNA was extracted with DNeasy blood and tissue kit (Qiagen, Germantown, Md.) and was RNase-treated. 100 ng of gDNA was treated with κ units of ECORV-HF (New England Biolabs, Ipswich, Mass.), 37° C., 15 minutes to cut the gDNA outside of the amplicon region. ddPCR forward and reverse primers (ddPCR F/R) were used to amplify a 210 bp amplicon. The assay was designed as a dual probe assay with WT-HEX and HDR-FAM probe run together and the reference-HEX probe was run in parallel in a separate well with the same ddPCR F/R primers using ddPCR supermix for probes (No dUTP, BIO-Rad). TABLE 3 and TABLE 4 lists primers and probes.

TABLE 3

| Primers | Forward (SEQ ID NO.) | Reverse (SEQ ID NO.) |
|---|---|---|
| HBB-1250 | AGGCTTTTTGTTCCCCCAGA | AGCCTTCACCTTAGGGTTGC |
| | (SEQ ID NO: 39) | (SEQ ID NO: 40) |
| SCL-386 | GGGTTGGCCAATCTACTCCC | CCTCTGGGTCCAAGGGTAGA |
| | (SEQ ID NO: 41) | (SEQ ID NO: 42) |
| ddPCR | CATAAAAGTCAGGGCAGAG | GTCTCCTTAAACCTGTCTTG |
| | (SEQ ID NO: 43) | (SEQ ID NO: 44) |
| LINC01206 | CAAAAAGCAAAATTTGGGGATA | CTTTTAGCCCAGTGCCAGAC |
| | (SEQ ID NO: 45) | (SEQ ID NO: 46) |
| MIR7974 | ATCAGCCCCTCTTTCTGGAT | AGTGCAGTGGTGCCATCATA |
| | (SEQ ID NO: 47) | (SEQ ID NO: 48) |
| HBD | CAGATCCCCAAAGGACTCAA | GCGGTGGGGAGATATGTAGA |
| | (SEQ ID NO: 49) | (SEQ ID NO: 50) |
| TULP4 | CAC GCCAGGATGTAAGCTCT | TCTGAGGCAAAAGTGCAAGA |
| | (SEQ ID NO: 51) | (SEQ ID NO: 52) |
| DENND3 | GGGGGTTTCTATCCCTCACT | CAAGAGGGTCAGGTTGAGGA |
| | (SEQ ID NO: 53) | (SEQ ID NO: 54) |
| HBB- | TCGTCGGCAGCGTCAGATGTGT | GTCTCGTGGGCTCGGAGATGTG |
| Miseq | ATAAGAGACAGGGGTTGGCCAA | TATAAGAGACAGCCTCTGGGTC |
| (with | TCTACTCCC | CAAGGGTAGA |
| adapter) | (SEQ ID NO: 55) | (SEQ ID NO: 56) |
| HBD- | TCGTCGGCAGCGTCAGATGTGT | GTCTCGTGGGCTCGGAGATGTG |
| Miseq | ATAAGAGACAGCACAAACTAAT | TATAAGAGACAGTCTACACATG |
| (with | GAAACCCTGCT | CCCAGTTTCCA |
| adapter) | (SEQ ID NO: 57) | (SEQ ID NO: 58) |

TABLE 4

| Probes | Sequence (SEQ ID NO.) |
|---|---|
| GTC HDR FAM | CTCCTGTCGAGAAGTCTGC |
| | (SEQ ID NO: 59) |
| GAA HDR FAM | CTCCCGAAGAGAAGTCTGC |
| | (SEQ ID NO: 60) |
| GTG HDR FAM | CTCCTGTGGAGAAGTCTGC |
| | (SEQ ID NO: 61) |
| GAG WT HEX | TGACTCCTGTCGAGAAGT |
| | (SEQ ID NO: 62) |
| REF HEX | GTTCACTAGCAACCTCAAACAGACACC |
| | (SEQ ID NO :63) |

The droplets were generated and amplified on a BIO-RAD thermocycler (95° C.: 5 min, 94° C.: 30 sec, 56° C.: 1 min, 72° C.: 1 min, go to step 2: 49 cycles, 98° C.: 10 min, 12° C.: ∞). The FAM and HEX fluorescence intensity were measured on the BIO-RAD QX200 machine (BIO-RAD, Hercules, Calif.). The HDR (%) events (HDR-FAM⁺) and WT (WT-HEX⁺) events were calculated after correction for the reference gene (REF-REX⁺, TABLE 3).

Measuring INDEL frequencies: gDNA from day 10 post-electroporation was used to amplify 1250 bp amplicon around the cut site with forward and reverse primers (HBB-F/R-1250, TABLE 3). The PCR products were cleaned using NucleoSpin gel and PCR clean-up kit (Machery Nagel, Bethlehem, Pa.) and subject to Sanger sequencing with the sequencing primer (SCL-F/R-386, TABLE 3). The sequences were analyzed using the TIDE/ICE algorithm to measure INDELs following editing.

MiSeq Analysis: the HBB (386 bp) and HBD (301 bp) gene-specific amplicons were amplified from 200 ng of gDNA using PrimeSTAR GXL DNA polymerase (TaKaRa, Kusatsu, Japan) with MiSeq primers (TABLE 3). The primers added an overhang adapter sequence onto the amplicons. Nextera 96-index kit (FC-121-1012, Illumina, San Diego, Calif.) was used to add a 5′ and 3′ unique index to each sample. The samples were purified with Agencourt AMPure XP (Beckman Coulter, Brea, Calif.) and the band verified on an agarose/PAGE gel. The samples were measured and pooled to make libraries and quality control was done on Qubit (ThermoFischer Scientific, Waltham, Mass.) and analyzed on MiSeq 500 CycleV2 kit (Illumina, San Diego, Calif.). The data was mined using the Crispresso2 algorithm. HBB analysis was used for on-target gene modification and HBD was used for Off-target analysis.

Engraftment studies in NBSGW mice: NOD, B6, SCID Il2rγ−/− Kit(W41/W41) (NB SGW) mice were purchased from Jackson Laboratories and maintained in a designated pathogen-free facility. All animal studies were performed according to the Association for Assessment and Accreditation of Laboratory Animal Care standards and were approved by the SCM Institutional Animal Care and Use Committee.

6-7-week-old NB SGW mice were busulfan (Selleckchem) treated 24 hours before transplant of edited cells. 2×10⁶ edited cells were infused by tail vein 24 hours after editing. The animals were monitored regularly. The BM and spleen from these animals were harvested at 3 weeks and 12-14 weeks after transfer and the cells were analyzed for human chimerism hCD45⁺, mCD45⁺ and multi-lineage engraftment of CD19⁺, CD33⁺, CD235⁺, CD3⁺, CD34⁺, CD38⁺ cells. The gDNA from BM cells were harvested and analyzed by ddPCR to determine HDR (%) and WT (%). The Indels were analyzed by TIDE/ICE sequencing. The BM cells were cultured in erythroid differentiation media for two weeks after harvest. The cells from ex vivo differentiation cultures were measured for CD235⁺ expression by flow cytometry. The cells were also pelleted, washed and analyzed by RP-HPLC at 2 weeks post-harvest to look for globin expression. BM cells (30,000 cells/plate/3 ml of methocult) were added to methocult complete media (STEMCELL technologies, Vancouver, Canada) and plated for CFU analysis. Single BFU-E colonies were picked at 14 days post-harvest, lysed in water and analyzed by IEC for globin expression.

Statistical Analysis: The data collected from experiments were analyzed on Graph Pad Prism 7 using two-way ANOVA analysis with Dunnett's multiple comparisons test. All samples across groups were compared to control or mock treated cells to evaluate significance. ns: not significant, * p<0.05, ** p<0.01, *** p<0.001, **** p<0.0001.

Erythroid cell lysis: erythroid cells cultured in differentiation media for 14 days were collected and washed in PBS to remove contaminating proteins. A hypotonic lysis of cells in HPLC grade water was performed. The supernatant of hemolysates were centrifuged at 20,000 g for 30 minutes at 4° C. and 1-10 μg of protein were injected into columns.

RP-HPLC analysis of erythroid cells: following erythroid differentiation, the expression of globin sub-types was assessed by RP-HPLC on a Shimadzu Prominence UFLC chromatograph using an Aeris 3.6 um Widepore C4 250×4.6 mm column (Phenomenex). Mobile phases used were: A: Water 0.1% TFA (trifluoroacetic acid), B: Acetonitrile 0.08% TFA at a flow rate of 0.8 ml/min. A gradient from 39% to 50% B was run over a 75-minute timed program. The column oven temperature was 40° C. and the sample tray was at kept at 4° C. The peaks were detected at 220 nm. A reference was run to compare the elution times of various globin peaks.

IEC of erythroid cells: the cells after PBS wash were analyzed on PolyCATA 200×2.1 mm 5 μm 1000 Å (PolyC #202CT0510) using the mobile phases: Phase A: Tris 40 mM, KCN 3 mM, in HPLC grade water adjusted to a pH 6.5 with acetic acid, Phase B: Tris 40 mM, KCN 3 mM in HPLC grade water, NaCl 0.2 M adjusted to a pH 6.5 with acetic acid. A timed 24-minute program was used to create a 2% to 100% B gradient with a flow rate of 0.3 mL/min. The column oven temperature was 30° C. and the sample tray was at kept at 4° C. The peaks were detected at 418 nm. A reference was run to compare the elution times of globin tetramers.

Colony sequencing: a 1250 bp amplicon around the cut site was amplified with HBB-1250 forward and reverse primers (TABLE 3) from 50 ng of gDNA using GXL DNA polymerase (Takara Bio). The PCR product was purified using the NucleoSpin Gel and PCR Clean-up kit (Macherey-Nagel, Bethlehem, Pa.) and subcloned into Zero Blunt TOPO PCR Cloning vector (Fisher Scientific, Hampton, N.H.) and transformed into TOP10 competent cells (Fisher Scientific, Hampton, N.H.). Kanamycin-resistant colonies were picked and sequenced with SCL-386 primer. Individual sequences were analyzed to determine if sequences were WT, NHEJ or HDR outcome.

T7-endonuclease assay: a 1250 bp region around the nuclease cut site was amplified from total gDNA using HBB-1250 primers using GXL DNA polymerase (Takara Bio). The PCR product was purified using NucleoSpin Gel and PCR Clean-up kit (Macherey-Nagel, Bethlehem, Pa.). 400 ng of PCR product was denatured and re-annealed in 1× Buffer 2 (New England Biolabs, Ipswich, Mass.) in 19 ul reaction volume. The samples were treated with T7 endonuclease I (New England Biolabs, Ipswich, Mass.) and incubated at 37° C. for 15 minutes and then loaded on a 1% agarose gel and imaged.

Flow cytometry and analysis: Flow cytometric analysis was done on an LSR II flow cytometer (BD Biosciences) and data analysis were done using FlowJo software (TreeStar). The gates were drawn on FSC/SSC populations corresponding to live cells and Singlets drawn using FSC-A/FSC-W.

ssODN design: single stranded oligonucleotides (ssODNs) were commercially synthesized by IDT (Ultramer® DNA Oligonucleotides) with phosphorothioate linkages in 2 terminal nucleotides on the 5′ and 3′ end. TABLE 5 lists ssODN sequences used for HDR.

TABLE 5

| ssODN | |
|---|---|
| sequences | SEQUENCES (SEQ ID NO.) |
| E6V GAG > GTC | TCAGGGCAGAGCCATCTATTGCTTACATTTGCTTCTGACACAACT |
| | GTGTTCACTAGCAA |
| | (SEQ ID NO: 64) |
| | CCTCAAACAGACACCATGGTGCATCTGACTCCTGTCGAGAAGTC |
| | TGCCGTTACTGCCCT |
| | (SEQ ID NO: 65) |
| | GTGGGGCAAGGTGAACGTGGATGAAGTTGGTGGTGAGGCCCTG |
| | GGCAGGT |
| | (SEQ ID NO: 66) |
| E6V GAG > CCC GAA | TCAGGGCAGAGCCATCTATTGCTTACATTTGCTTCTGACACAACT |
| | GTGTTCACTAGCAA |
| | (SEQ ID NO: 67) |
| | CCTCAAACAGACACCATGGTGCATCTGACTCCCGAAGAGAAGTC |
| | TGCCGTTACTGCCCT |
| | (SEQ ID NO: 68) |
| | GTGGGGCAAGGTGAACGTGGATGAAGTTGGTGGTGAGGCCCTG |
| | GGCAGGT |
| | (SEQ ID NO: 69) |
| E6V GAG > GTG | TCAGGGCAGAGCCATCTATTGCTTACATTTGCTTCTGACACAACT |
| | GTGTTCACTAGCAA |
| | (SEQ ID NO: 70) |
| | CCTCAAACAGACACCATGGTGCATCTGACTCCTGTGGAGAAGTC |
| | TGCCGTTACTGCCCT |
| | (SEQ ID NO: 71) |
| | GTGGGGCAAGGTGAACGTGGATGAAGTTGGTGGTGAGGCCCTG |
| | GGCAGGT |
| | (SEQ ID NO: 72) |

Underlined sequences are changes from wild-type sequences

### Nuclease Efficiency in CD34 Cells

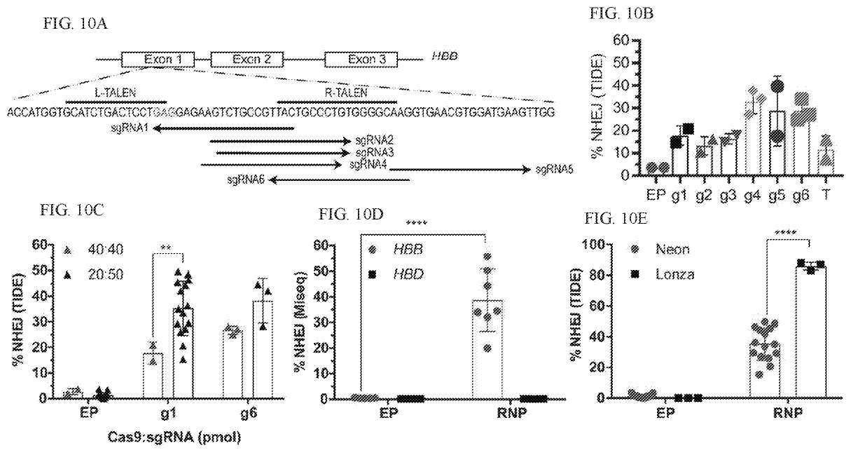

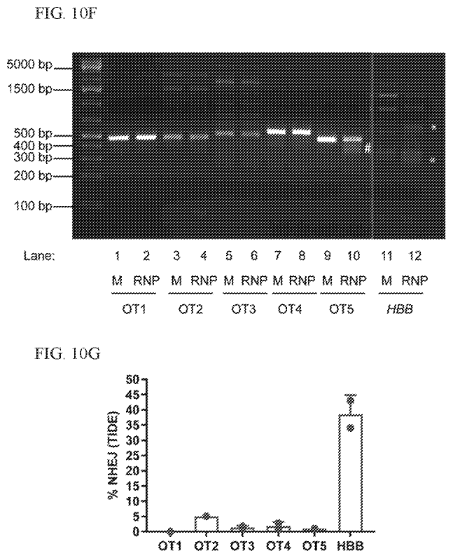

A nuclease screen was conducted to identify methods to efficiently create double-stranded breaks (DSBs) within exon 1 of HBB gene (FIG. 10A). The cleavage efficiencies of alternative sgRNAs delivered as RNP complexes or TALEN-based nucleases was evaluated in CD34+ mPBSCs. In the initial nuclease screen, RNP delivery of a series of candidate sgRNAs was tested at a Cas9: sgRNA ratio of 1:1 and identified Guide 4 (g4), g5, g6, g1 as most efficient at creating DSBs (FIG. 10B). Based on these findings, sgRNA-g1 was optimized, as it created a DSB adjacent to codon 6, the site of the SCD mutation. sgRNA-g6 (G10) was also extensively tested in parallel. Upon testing both guides at a Cas9: sgRNA ratio of 1:2.5 with the neon electroporation system, total editing rates doubled for sgRNA-g1 (g1: increased from 17.8±4.4% to 35.2±10.6%; g6: 26.7±1.6% to 38.3±8.7%, FIG. 10C). The on-target HBB disruption for sgRNA-g1 by MiSeq analysis was 38.7±12.2% (n=7 donors) and off-target HBD disruption was 0.129±0.01% (FIG. 10D). The top 5 off-target genes predicted by CCTop 22 for sgRNA-g1 showed no Indels by T7 endonuclease assay (FIG. 10F) and TIDE sequencing (FIG. 10G). Overall editing rates increased by ˜2.5-fold with use of a nucleofection system (Lonza; 86±2.6%, n=3 donors) compared to the electroporation system (Neon; 35.2±10.6%, n=15 donors, FIG. 10E).

Introducing a GTC Change Through rAAV6 Donor Template Delivery

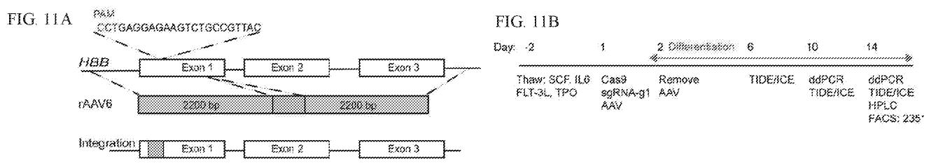

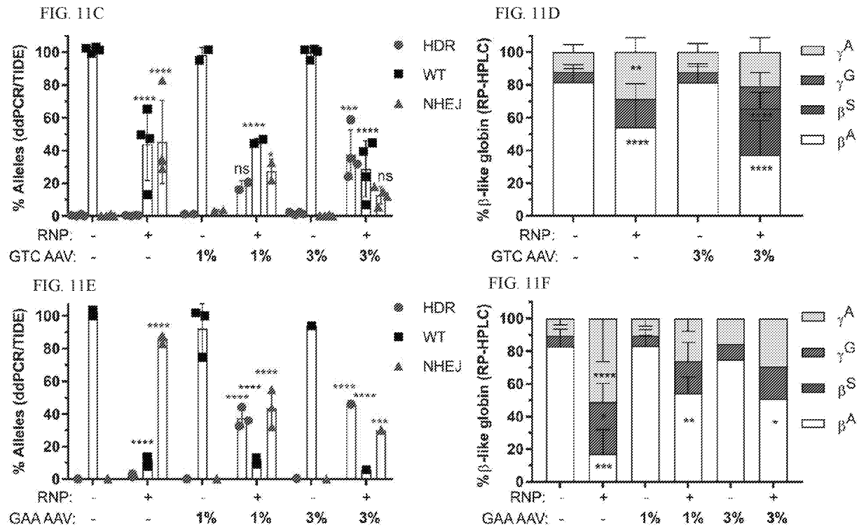

An rAAV6 vector was constructed with 2.2 kb homology arms designed to introduce either a GTC (encoding E6V) or a silent change GAA (encoding E6optE) at codon 6 of exon 1 of the HBB gene. The design was focused on preserving intron 1 and native promoter/enhancer regions to maximize transcription and translation (FIG. 11A). The experimental timeline is shown in FIG. 11B. Testing 3% GTC (encoding E6V) rAAV6 donor template following RNP-mediated cleavage resulted in HDR rates of 37.5±15% and residual NHEJ rates of 12.7±5.3% (FIG. 11C). Testing of GTC (encoding E6V) rAAV6 with both the electroporation and nucleofection systems demonstrated that increases in total editing rates lead to increases in rates of both HDR and residual NHEJ (FIG. 11G). The HDR and residual NHEJ rates measured when RNP is co-delivered with GTC rAAV6 were additionally validated by colony sequencing (30.8±6.3% HDR, 17.9±7.2% NHEJ, n=5 donors, FIG. 11H). Cell viability after electroporation and transduction with 3% culture volume of GTC rAAV6 was on an average 79.2% (FIG. 11I). Globin sub-types were measured in differentiated erythroid precursors by Reverse Phase HPLC (RP-HPLC). Editing alone and editing in the presence of GTC rAAV6 lead to a significant decrease in βA (82% to 54±16%, using neon) and 2-fold increase in γA (HBG1) and γG (HBG2). Co-delivery of RNP and 3% GTC rAAV6 lead to 28.4±9.8% βS expression (n=6 donors, FIG. 11D). The globin tetramers in erythroid cells generated following co-delivery of RNP and GTC rAAV6 (encoding E6V) were measured by ion-exchange chromatography (IEC). A decrease in HbA and a dose-dependent increase in HbS tetramers (15.8%) was observed with co-delivery of RNP and 3% GTC rAAV6 (Figure S2D). A sample chromatogram of GTC (encoding E6V) rAAV6 treated cells by RP-HPLC analysis confirmed the presence of 38.6% βs peak (FIG. 11K).

In parallel with these nucleotide change studies, extensive testing was also performed of more complex rAAV6 donor template constructs designed to introduce a GFP expression cassette in association with alternative tissue-specific enhancers and regulators (FIG. 11L). These latter rAAV6 donor templates resulted in significant HDR rates (1321: 14.4% and 1322: 18.4%, FIG. 11M), but the desired globin expression was compromised (with βs=1.35 to 2.6%, FIG. 11N).

Introducing the GAA SNP Change Through rAAV6 Donor Template Delivery

Introduction of the sickle mutation in normal cells does not assess the potential to revert the mutation in patient cells and might also alter the fitness of edited erythroid progenitors. In lieu of studies using HSC from SCD subjects, the introduction of a silent SNP change (GAA; encoding E6optE) was tested. Testing of GAA (encoding E6optE) rAAV6 donor template (1% culture volume) co-delivered with RNP resulted in HDR rates of 37.5±6% and NHEJ rates of 43.7±11.5% (FIG. 11E). Of note, the increase in total NHEJ events using co-delivery of RNP and GAA (encoding E6optE) rAAV6 compared with the co-delivery of RNP and GTC (encoding E6V) rAAV6 cassettes likely reflect an increase in overall editing rates using the nucleofection system. While the data for GTC (encoding E6V) rAAV6 editing included experiments using both Neon (n=3) and Lonza (n=1), GAA (encoding E6optE) rAAV6 co-delivery was tested exclusively with the Lonza (n=3). Cell viability after electroporation and transduction with 1% GAA (encoding E6optE) rAAV6 was on an average 60% (FIG. 11I). RP-HPLC analysis identified a marked decrease in βA levels (from 82% in control cells to 16.9±15% in RNP edited cells) and a 3-fold increase in βA (HBG1) and γG (HBG2) in the RNP-edited samples. In contrast, co-delivery of RNP and 1% GAA (encoding E6optE) rAAV6 lead to a less robust reduction in βA levels (54.2±10% βA expression; n=3, FIG. 11F) and a less prominent increase in βA (HBG1) and γG (HBG2). The retention of βA expression following co-delivery of RNP and GAA (encoding E6optE) rAAV6 can be ascribed to AAV-mediated HDR. Consistent with this conclusion, RP-HPLC analysis of cells treated with co-delivery of RNP and GAA (encoding E6optE) rAAV6 showed 64.7% βA after HDR (FIG. 11O). Taken together, our findings demonstrate the capacity of RNP and rAAV6 co-delivery to promote high-levels of HDR in exon 1 of HBB leading to either an introduction of sickle mutation or a silent mutation designed to revert the sickle mutation in patient cells.

Introducing the GTC Change Through ssODN Donor Template Delivery

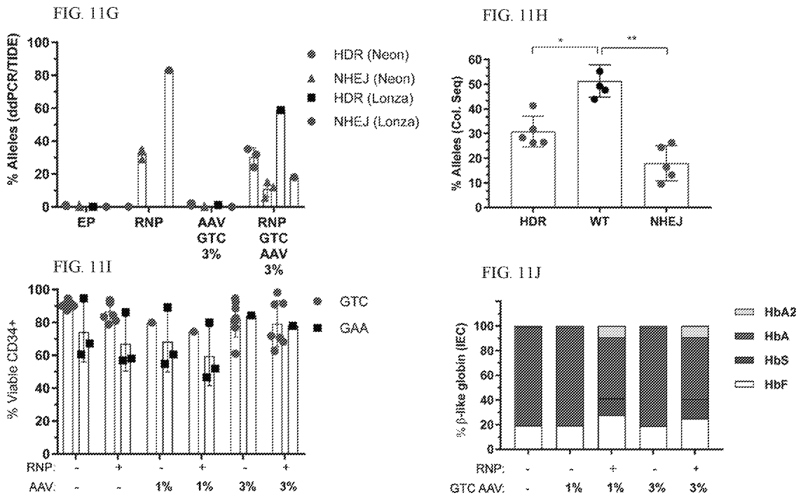

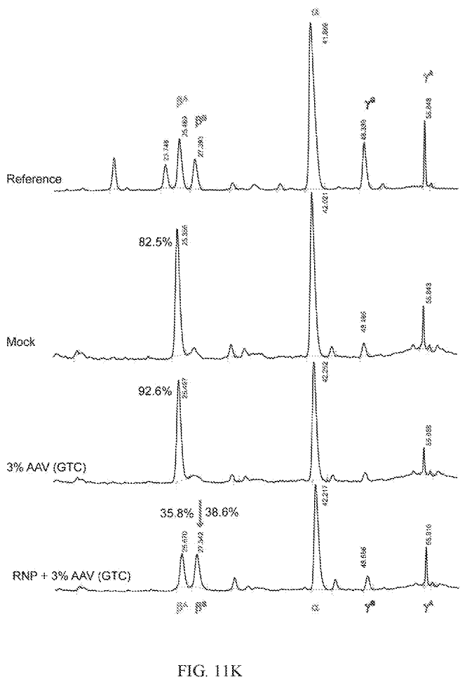

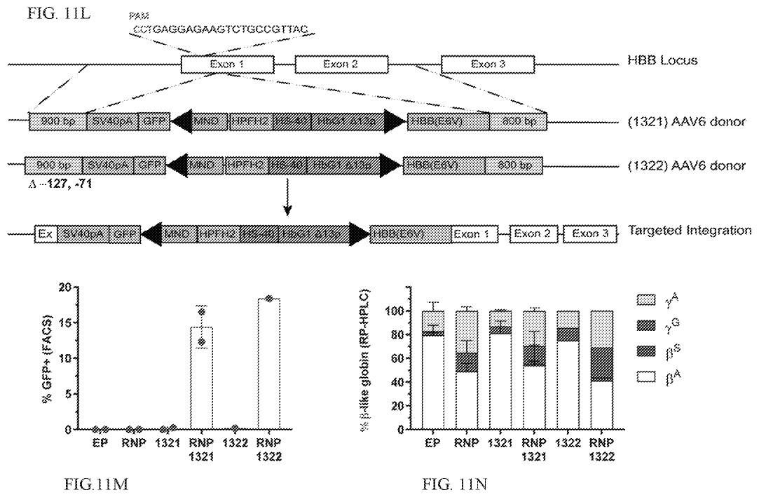

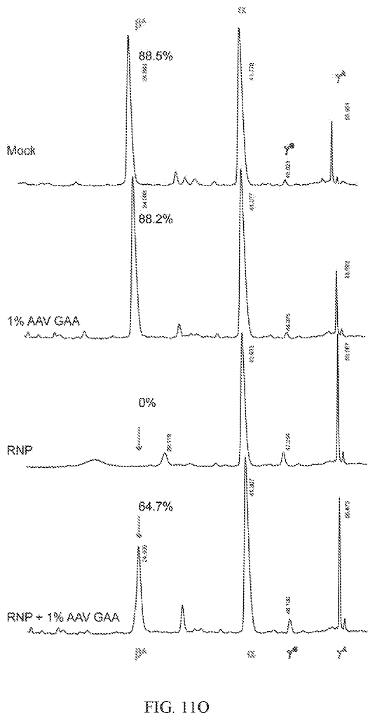

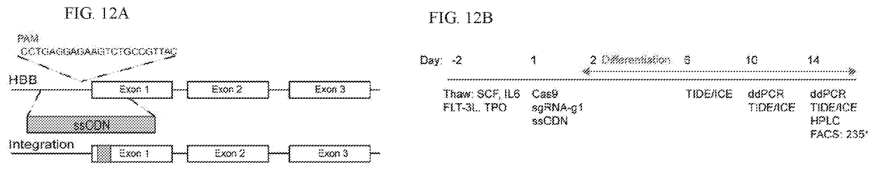

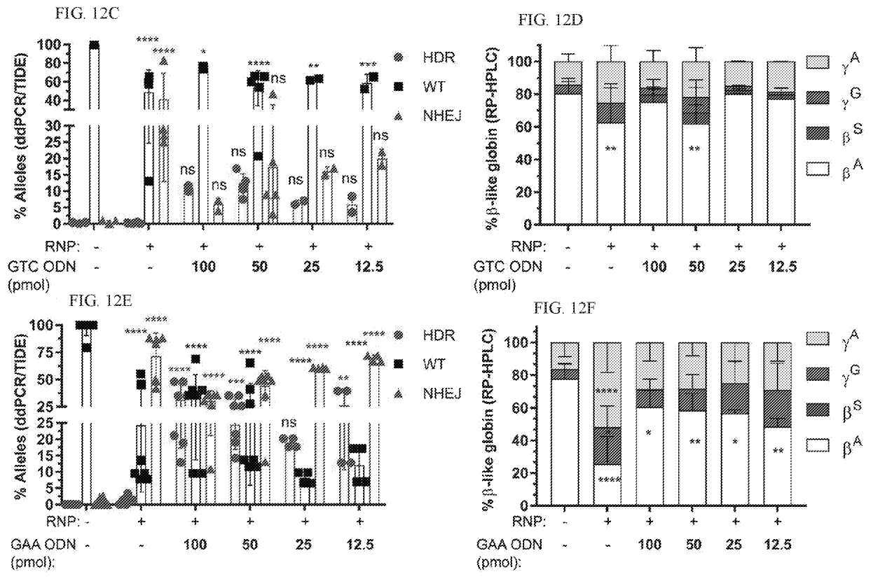

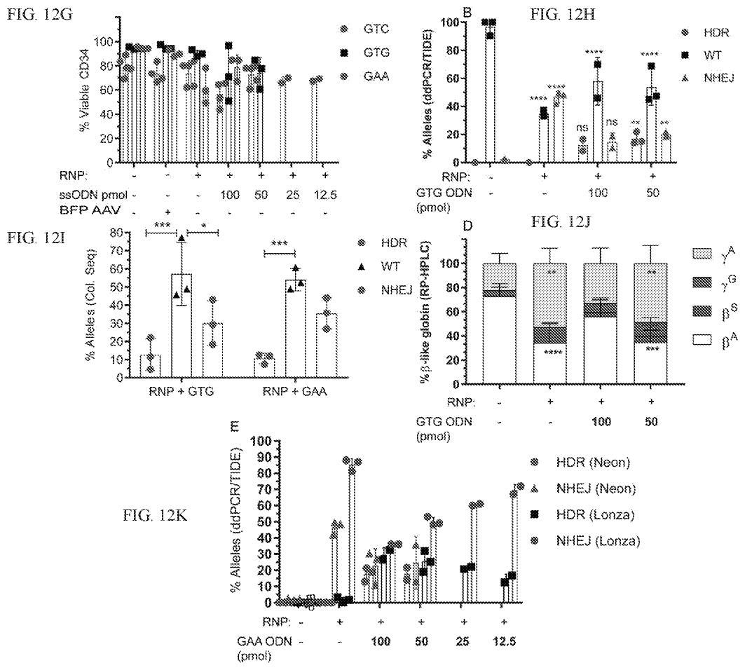

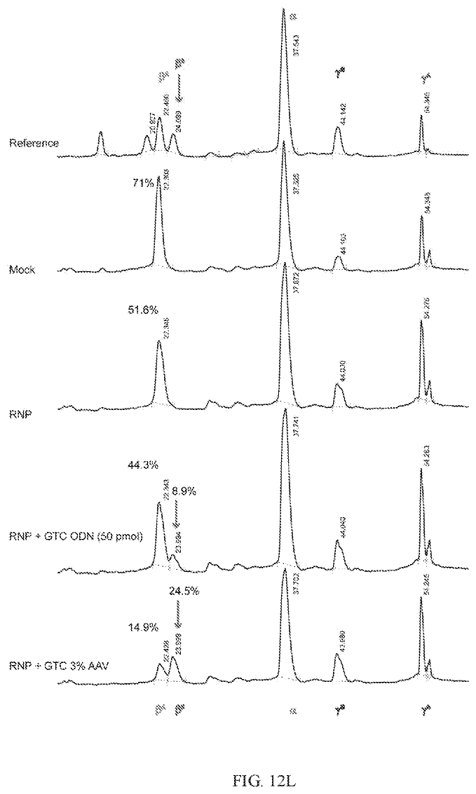

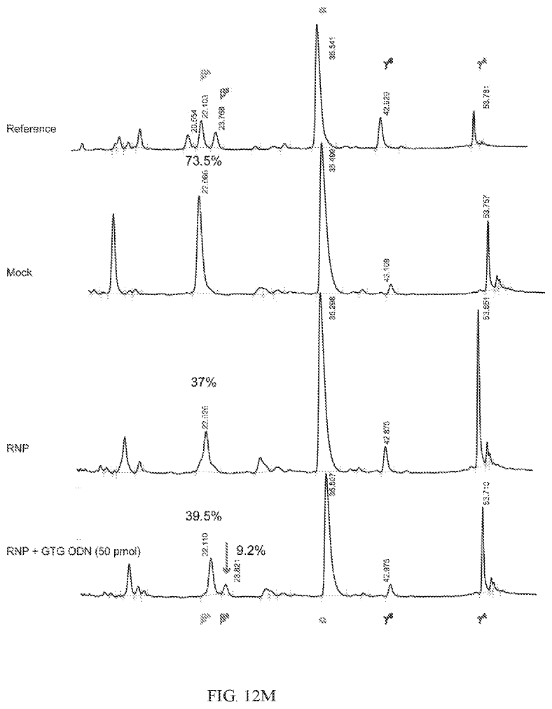

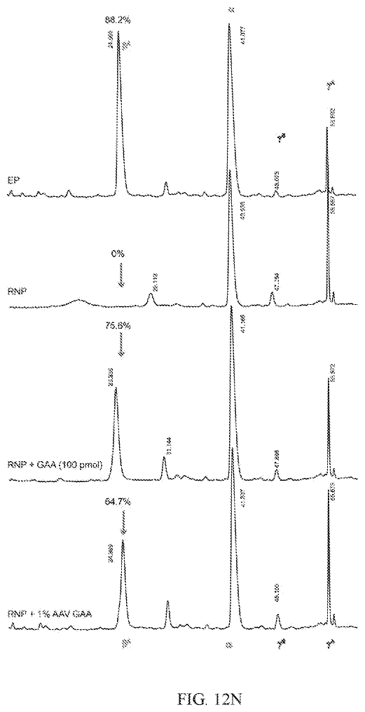

Studies were performed assessing the efficiency of co-delivery of RNP and ssODNs to introduce the identical nucleotide changes achieved using rAAV6 in mPBSCs. Alternative 168 bp ssODNs were designed to generate either a GTG (encoding E6V), GTC (encoding E6V) or GAA (encoding E6optE) nucleotide change (FIG. 12A). The experimental timeline is shown in FIG. 12B. There was a dose-dependent increase in cytotoxicity with increasing concentrations of ssODN tested (FIG. 12G). The HDR gene conversion rate following co-delivery of RNP and 50 pmol of ssODN was 11.9±3.4% for the GTC ODN and 17±4.3% for the GTG ODN and the residual NHEJ was 17.4±17.5% and 20.0±1.7% respectively (FIG. 12C, FIG. 12H). The HDR and NHEJ rates for 50 pmol of GTG ssODN (encoding E6V) were further validated by colony sequencing and were 12.6±8.8% and 30.1±12.4%, respectively (FIG. 12I). Globin sub-types were assessed by RP-HPLC. A significant decrease was observed in βA and a 1.5-fold increase βA (HBG1) and γG (HBG2) following RNP-mediated disruption. The co-delivery of RNP and varying concentrations of ssODN led to a dose-dependent increase in sickle globin expression with optimal βS expression with 50 pmol of GTC (encoding E6V) ssODN (FIG. 12D) and GTG ssODN (FIG. 12J). Editing with GTC (encoding E6V) ssODN resulted in 5.2% βS expression (50 pmol, n=5, FIG. 3D) and GTG (encoding E6V) ssODN resulted in 5.3% βS expression (50 pmol, n=3) respectively (FIG. 12J). Consistent with these averages, a sample chromatogram derived from differentiated erythroid cells demonstrated 8.9% βS expression with GTC (encoding E6V) ssODN (FIG. 12L) and 9.2% βS with GTG (encoding E6V) ssODN (FIG. 12M). A direct comparison of editing in mPBSCs from the same donor using GTC ssODN vs. rAAV6 demonstrated 8.9% vs. 24.5% βS expression, respectively (FIG. 12L).

Introducing the GAA SNP Change Through ssODN Donor Template Delivery

Consistent with the studies using rAAV6 donor templates, introduction of an alternative, silent SNP change (GAA; encoding E6optE) was tested. Co-delivery of RNP and 50 pmol of GAA (encoding E6optE) ssODN resulted in HDR gene conversion rate of 24.5±7.6% with residual NHEJ rates of 44±13.8% (FIG. 12E). With increasing concentration of ssODN, a dose-dependent increase in HDR and a corresponding decrease in NHEJ was observed with both the Neon and the Lonza systems (FIG. 12K). Editing outcomes following use of the Neon electroporation system were also validated by colony sequencing demonstrating HDR rates of 10.6±2.8% and residual NHEJ rates of 35.5±8.6% (FIG. 12I). Globin sub-types in differentiated erythroid pellets were measured and a significant decrease in βA (25.7%, n=6 donors) and a 1.5 to 3-fold increase βA (HBG1) and γG (HBG2) was observed with RNP-mediated disruption. In contrast, use of GAA (encoding E6optE) ssODN donor template retained βA expression at 58.4% (n=6 Donors, FIG. 12F). A sample chromatogram showing globin sub-types in edited differentiated erythroid cells demonstrate an increase from 0% HbA in RNP disrupted samples to 75.6% following co-delivery of RNP and GAA (encoding E6optE) ssODN (FIG. 12N). A direct comparison of RNP-only edited cells to mPBSCs edited using co-delivery of RNP and HDR donor template showed an increase in HbA expression from 0% to 75.6% and 64.7% for GAA ssODN and rAAV6 donor templates respectively (FIG. 12N).

Comparison of ssODN and rAAV6 Donor Template Delivery Methods by MiSeq Analysis

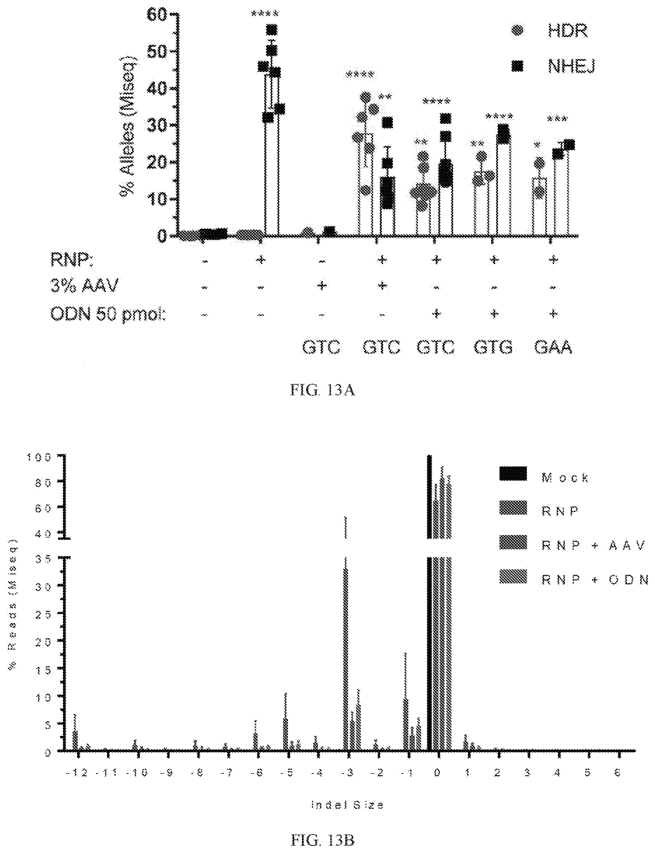

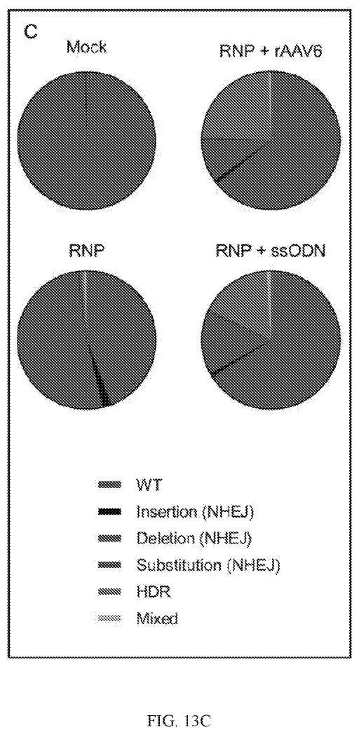

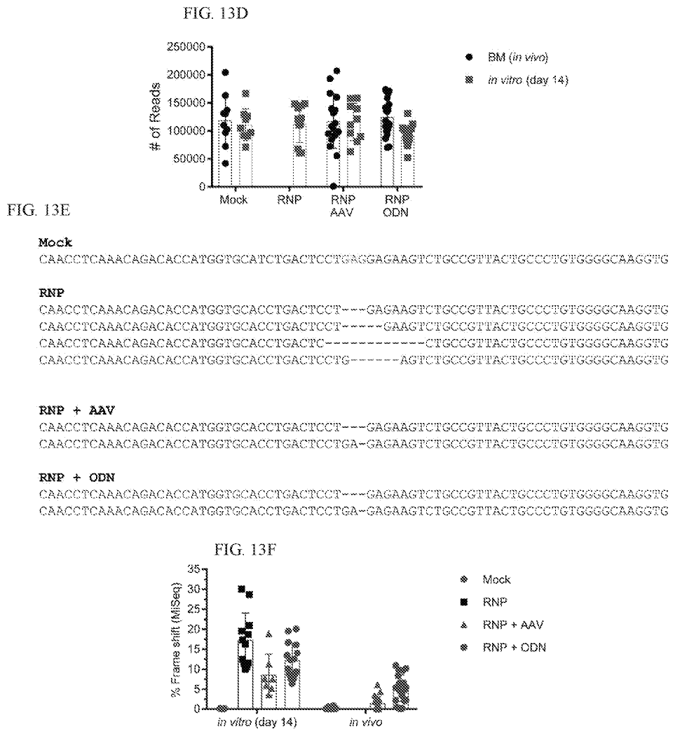

To further assess gene editing efficiencies achieved using our alternative platforms, MiSeq analysis was used to validate the editing outcomes. The HDR and NHEJ rates achieved using the Neon electroporation system for RNP co-delivery in association with all ssODN donor templates vs GTC (encoding E6V) rAAV6 donor templates were assessed. An average of 113,000 pair-wise aligned reads were obtained from each in vitro (and in vivo) sample (FIG. 13D). The data demonstrated that rAAV6 donor template drove higher levels of HDR than NHEJ (GTC rAAV6: 27.8% HDR, 16% NHEJ) and that ssODN delivery drove higher levels of NHEJ than HDR (GTC ssODN: 14.3% HDR and 19.6% NHEJ) in vitro (FIG. 13A, FIG. 13B, FIG. 13C). Analyzing the indel spectrum produced, RNP alone resulted in 60.4% deletions (primarily −3, −1, −5, −6 and −12 bp deletions) along with 2% insertions. Co-delivery of a donor template with RNP decreased the indel spectrum to primarily −3 and −1 bp deletions (FIG. 13B, FIG. 13E). Wild type (WT), NHEJ with deletions and HDR alleles were observed in rAAV6-edited and ssODN-edited samples (FIG. 13C). Crispresso 27 analysis identified that rAAV6 donor template delivery resulted in fewer frame shift mutations (8.6% in vitro and 1.4% in vivo) compared to ssODN donor template delivery (in vitro 12.2% and 5.3% in vivo; FIG. 13F).

Impact of ssODN Vs. rAAV6 Delivery on Sustained Engraftment of HDR-Edited Cells In Vivo

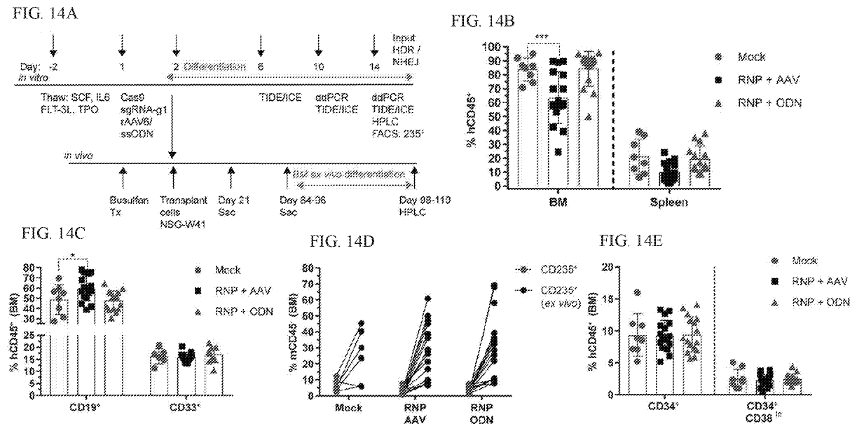

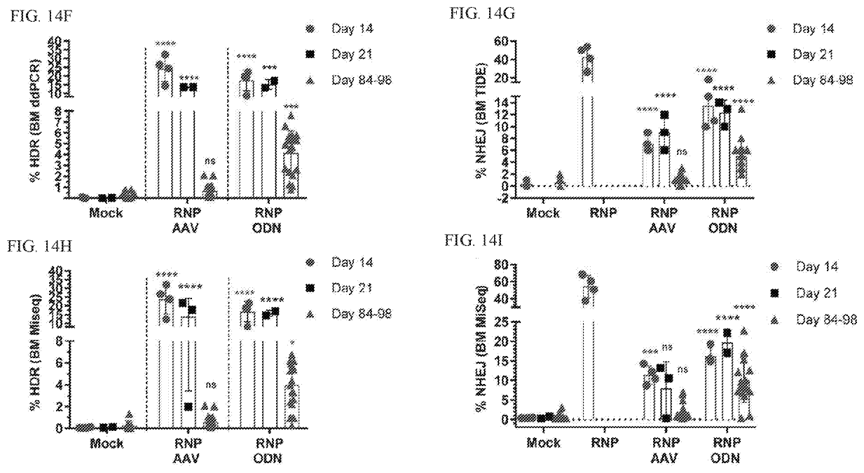

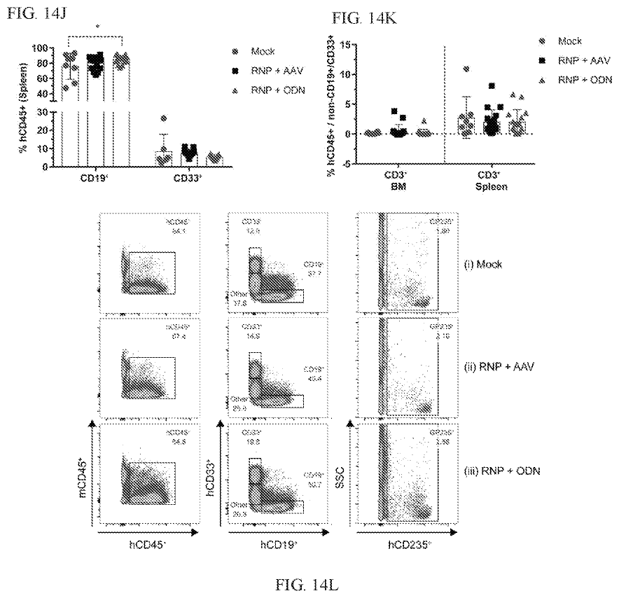

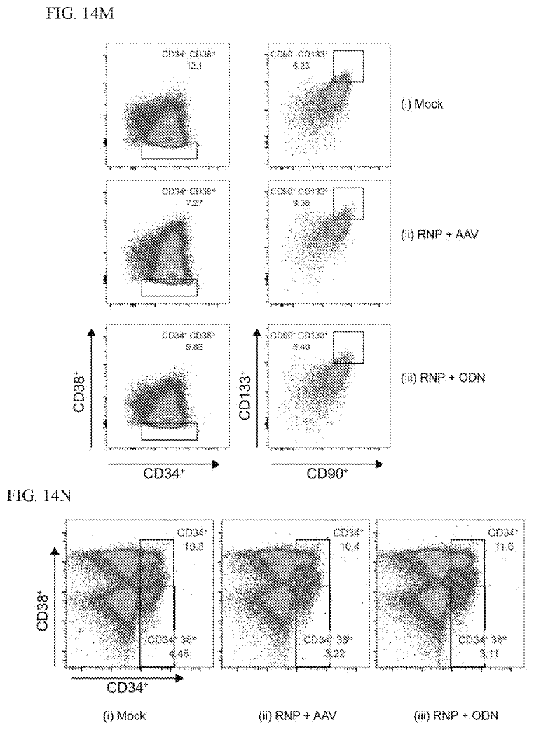

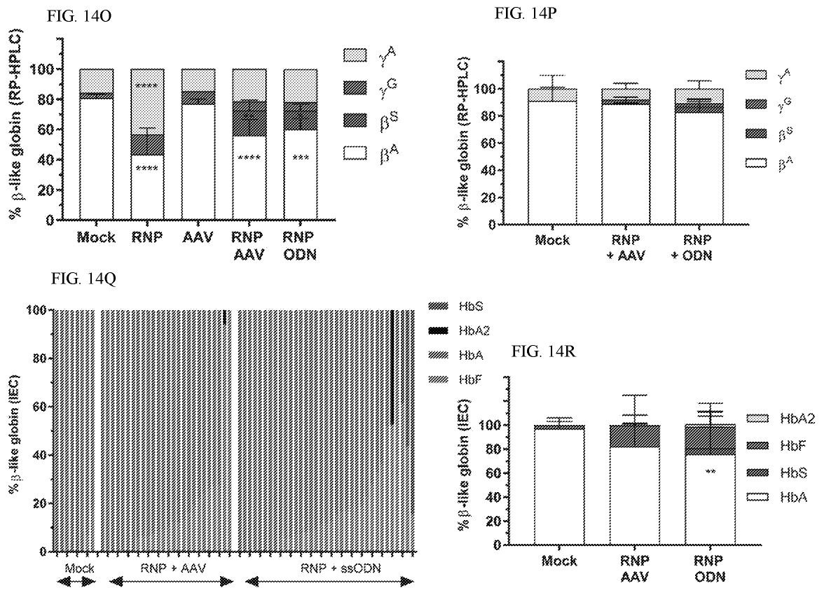

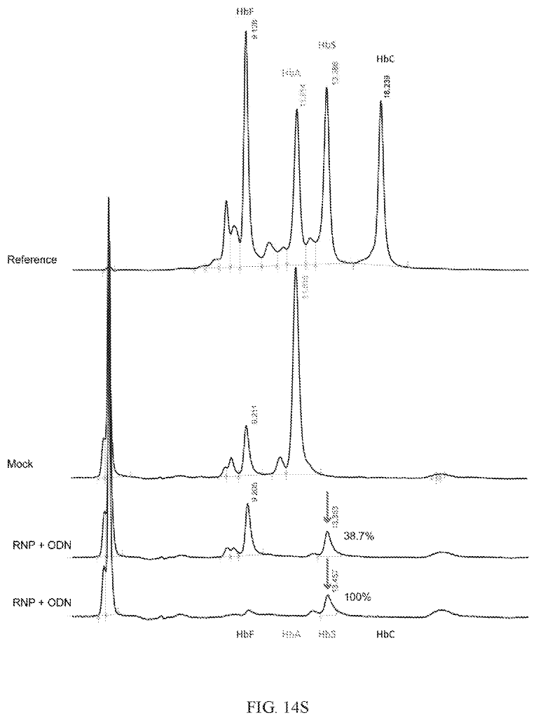

To understand the role of alternative donor template platforms in altering the long-term engraftment potential of HDR-edited CD34+ cells, healthy control mPBSCs edited to introduce the GTC (encoding E6V) change were transplanted into busulfan conditioned (12.5 to 25 mg/kg) NBSGW recipient mice, an immunodeficient strain that permits development of a human erythroid compartment. Cells, derived from identical donors, edited with each platform (2×10⁶ cells) were transplanted at Day 1 following electroporation. Transplanted animals were assessed over time and evaluated at 3 and 12-14 weeks for human cell engraftment in the BM and spleen (FIG. 14A).

Human chimerism was comparable for recipients of mock- or ssODN-edited cells. In contrast, a significant decrease in hCD45+ cell engraftment was observed in recipients of rAAV6-edited cells (FIG. 14B). Additionally, the proportion of CD19+ B cells was modestly increased in the rAAV6-edited group suggesting skewing towards more differentiated progeny (FIG. 14C). Other lineages including myeloid (CD33+), T cell (CD3+) and erythroid (CD235+) cells were represented equivalently across cohorts (FIG. 14C, FIG. 14D, FIG. 14J, FIG. 14K, FIG. 14L). Cells isolated from the BM were cultured in erythroid differentiation media for 2 weeks after harvest to permit expansion of CD235+ cells (with increase of 4.01% at harvest to 27.6% in ex vivo cultures; FIG. 14D). Representative flow plots of edited donor cells pre- and post-transplant revealed equivalent proportions of primitive HSCs sub-populations including: CD34+; CD34+CD38lo, and CD34+CD38loCD133+CD90+ cells (FIG. 14E, FIG. 14M, FIG. 14N). The input HDR rates (Day 14 in culture) across 4 transplants were 24.28±7.5% and 17.5±6% for rAAV6 and ssODN delivery methods, respectively. HDR edited cells in the BM at 3 weeks post-transplant declined to 13.58±0.16% and 15.19±2.8% (n=2) for rAAV6 and ssODN delivery, respectively. At 12-14 weeks, the HDR rates declined precipitously to 0.66±0.66% (n=17) in recipients of rAAV6 donor template edited cells. Strikingly, HDR rates also declined but to a much lesser extent to 4.136±2.1% (n=18) in recipients of ssODN donor template edited cells (FIG. 14F). The input NHEJ was 7±1.4% and 13.5±3.7% for rAAV6 and ssODN donor template delivery methods, respectively and remained unchanged at 3 weeks post-transplant (rAAV6: 9±3%, ssODN: 12.3±2.1%) and declined at 12-14 weeks (NHEJ rAAV6: 1.3±0.85%, ssODN: 5±2.7%, FIG. 14G). HDR and NHEJ rates in the BM in vivo were verified by MiSeq analysis (HDR: rAAV6: 0.65±0.65%, ssODN: 3.84±2.1%; NHEJ: rAAV6: 2.5±2.5% and ssODN: 9.9±5.3%, FIG. 14H, FIG. 14I).

A subset of each initial edited cell population was maintained in vitro in erythroid culture conditions and analyzed for globin sub-types. rAAV6-edited cells exhibited 16.4±6.8% and ssODN-edited cells 12.42±4.4% βS expression (FIG. 14O). The ex vivo BM cultures analyzed by HPLC expressed 3.8% βS (n=3 animals) in the ssODN-edited group. In contrast, βS was not detected in the rAAV6 (n=4 animals) or mock-edited samples (n=2 animals, FIG. 14P). HPLC of the 69 BFU-E colonies revealed 3/35 colonies derived from the ssODN-edited group expressing βS resulting in an average of 5.13% βS expression. In contrast, βS expression was not detected in rAAV6-edited (n=26 colonies) or mock-edited colonies (n=8 colonies, FIG. 14Q, FIG. 14R). Chromatograms of single erythroid colonies derived from the ssODN-edited group demonstrated βS expression levels of 38.7%, 84.5% and 56.3% (FIG. 14S, FIG. 14T). The HPLC profile of single colonies for mock samples contained 97% HbA whereas the edited groups had a decrease in HbA and an increase in HbF (rAAV6: 17.4%, ssODN: 17.9%) and/or HbS (FIG. 14Q, FIG. 14R, FIG. 14S, FIG. 14T). Taken together, these studies demonstrated that ssODN-modified cells outperformed rAAV6-modified cells in vivo leading to both higher sustained engraftment of HDR-edited cells and sickle globin expression.

Delivery of a DNA donor template is useful in achieving precise gene correction following targeted gene cleavage in human hematopoietic stem cells. The overall ratio of HDR to NHEJ impacted the potential clinical benefit of gene correction in sickle cell disease. In the studies disclosed herein, a role of alternative donor template was assessed for delivery methods to achieve initial gene conversion events in vitro as well as the impact on the survival, stem-like potential and sustained engraftment of edited cells in vivo. The combined data demonstrated the complexity and addressed some of the challenges in achieving long-term clinical gene correction in SCD. While no major differences in HSC viability were observed, phenotype or expansion in vitro using rAAV6 compared with ssODNs, rAAV6 donor templates were shown to mediate consistently higher HDR:NHEJ ratios. In contrast, in transplant experiments, much higher levels of sustained HDR were achieved using HSC edited with ssODN donor templates.

An initial screening was performed of a candidate TALEN pair and multiple candidate guide RNAs spanning a 53 bp region around the sickle mutation site. As shown herein, sgRNA-g1 efficiently created DSBs immediately adjacent to the sickle mutation site (between 21-22 bp) and was therefore chosen as a more useful guide than sgRNA-g6 (G10) which generated a DSB 16 bp away from the mutation site. Use of Cas9: sgRNA at a ratio of 1:2.5 promoted the highest levels of editing in human mPBSCs with no demonstrable off-target effects (FIG. 10D, FIG. 10F, FIG. 10G). Of note, total editing rates more than doubled when using a nucleofection system (FIG. 10E) with increases in both HDR as well as residual indels in vitro (FIG. 11G, FIG. 12G). Following delivery of RNPs containing sgRNA-g1, the capacity of alternative rAAV6 cassettes versus a series of ssODNs to drive a one or two nucleotide change in the sixth codon of exon 1 of HBB was tested. The in vitro studies demonstrated that rAAV6 promotes greater rates of HDR than NHEJ (GTC rAAV6: 37.5±15% HDR and 12.7±5.3% NHEJ, FIG. 11C). In contrast, ssODN delivery drives more NHEJ than HDR (GTC ssODN: 11.9±3.4% HDR and 17.4±17.5% NHEJ, FIG. 12C, FIG. 13A).

### Example 4—Comparison of CM149 and ER100 Lonza Methods

CM149 and ER100 Lonza nucleofection methods were compared with RNP editing followed by rAAV6 or ssODN donor template delivery. Viability (assessed using MUSE cell counter), HDR (assessed by ddPCR) and NHEJ (assessed by ICE) outcomes were compared with Lonza programs CM149 and ER100 using mobilized CD34+ HSC cultured in SCGM (1 million cells/ml) or SFEM-II media (250,000 cells/ml).

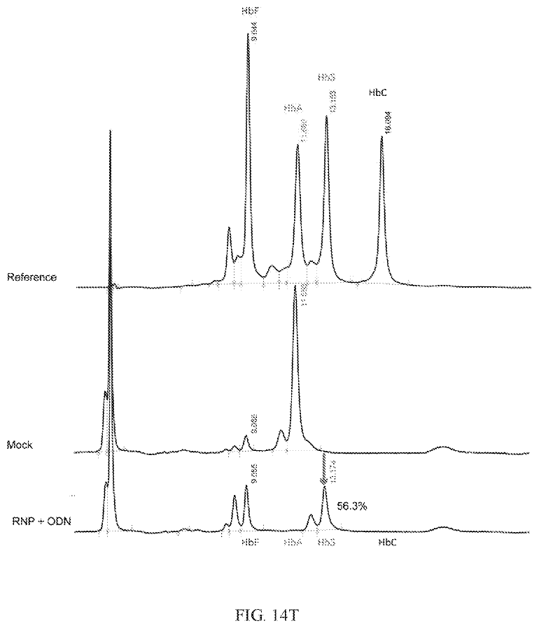

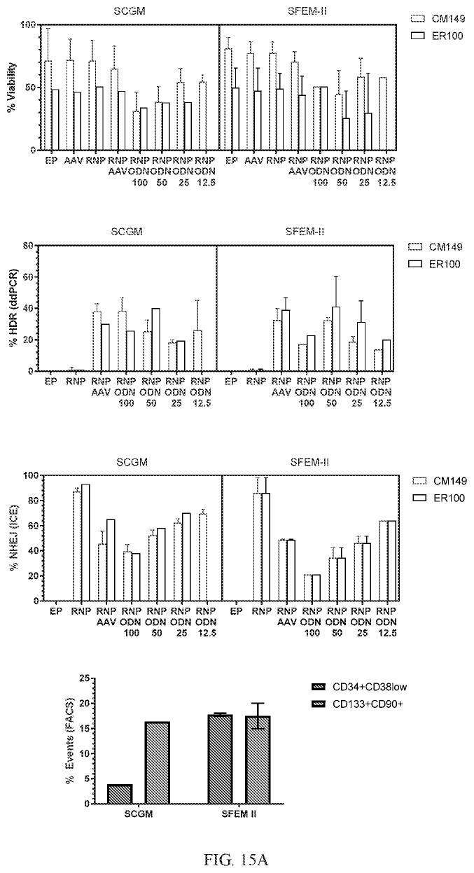

The NHEJ disruption rate for RNP alone and residual indels after HDR were comparable for the 2 Lonza methods in both media tested. In contrast, HDR-editing with RNP and 50 pmol and 25 pmol concentration of ssODN using the CM149 method led to more viable cells at Day 2 post-editing than the ER100 method (FIG. 15A). 40% HDR was achieved with RNP+50 pmol of ssODN in SFEM-II media. Although ER100 achieved higher HDR rate than CM149 with 50 pmol and 25 pmol of ssODN, the viability and cell counts were dramatically reduced using ER100 and therefore not desirable. SFEM-II media with low density culturing preserved more cells in the LT-HSC (CD34+CD38 Lo) compartment (CD34+CD38Lo) than the SCGM media and therefore was likely to be preferable for in vivo engraftment. Both methods drove approximately 30% βA adult globin expression in both media tested (FIG. 15B). Both methods led to viabilities below 70% post-editing (day 2) and therefore both Lonza methods are likely sub-optimal for long-term engraftment.

### Example 5—Comparison of Cell Density at the Time of Nucleofection

Cell density at the time of nucleofection was compared to evaluate the role of cell density in driving optimal HDR, NHEJ and viability. Cell density (200,000/400,000/600,000 cells per 20 μl nucleofection reaction) was varied in a 20 μl Lonza reaction to understand if number of cells had a role in viability, HDR and NHEJ outcomes after RNP editing and rAAV6 and ssODN donor template delivery.

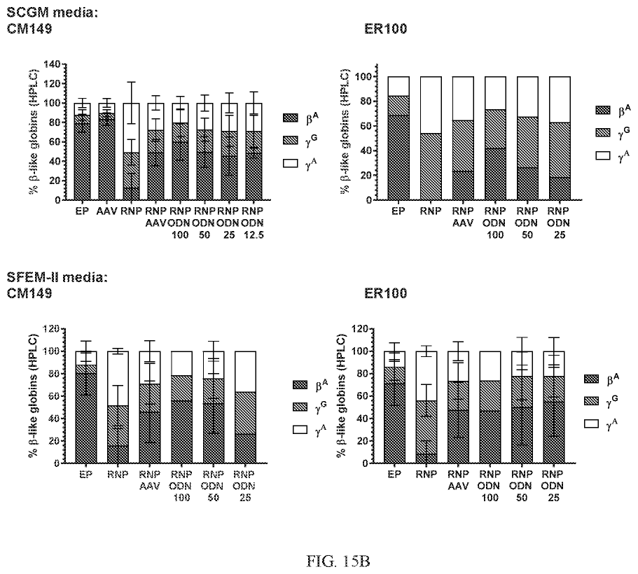

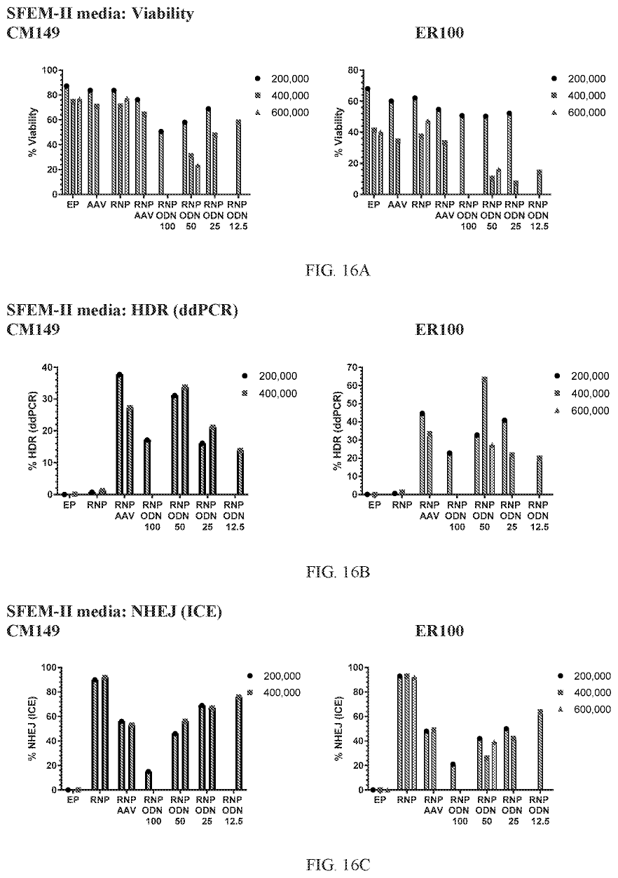

200,000 cells/20 μl nucleofection reaction outperformed the rest of the cell densities with respect to viability of CD34+ cells post-editing on day 2 after nucleofection with both methods (CM149 and ER100) tested (FIG. 16A). 200,000 cells/20 μl nucleofection reaction outperformed the rest of the cell density with respect to HDR as measured on day 14 post-editing, except RNP+50 pmol of ssODN tested with ER100 where 400,000 cells/20 μl reaction worked the best (FIG. 16B). The cell density did not have an impact on NHEJ rates (FIG. 16C). Total NHEJ rates and residual NHEJ rates after HDR were comparable across various densities with both methods tested.

### Example 6—Assessment of Cell Viability and HDR Rates

Cell viability and HDR rates were assessed using alternative Lonza programs in association with RNP and ssODN delivery.

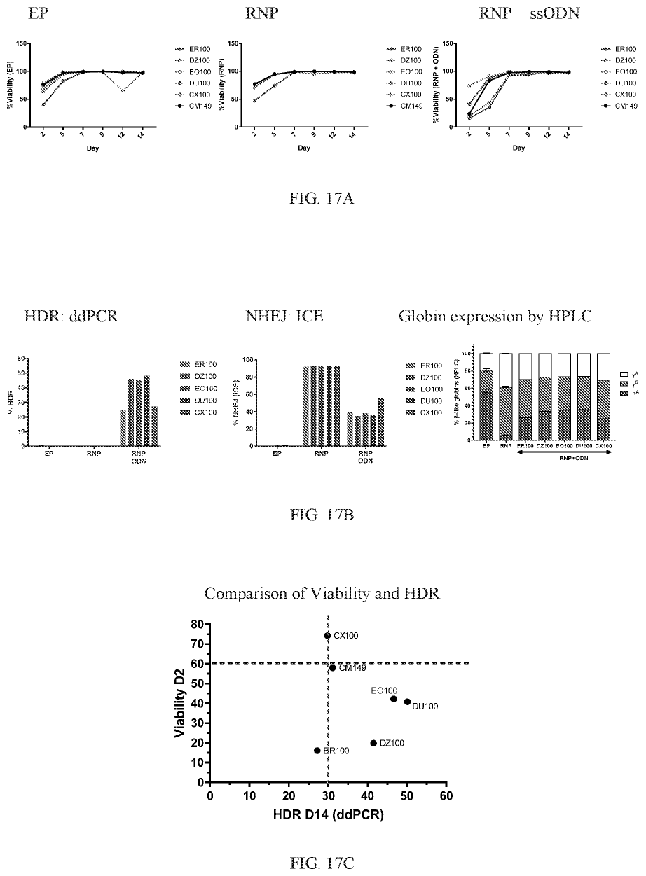

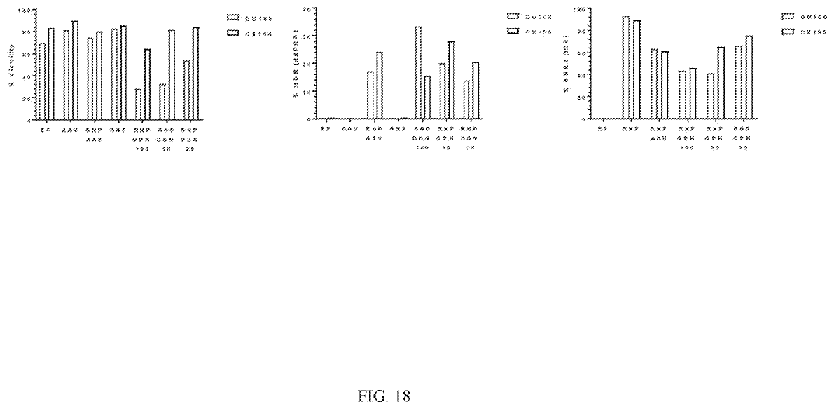

CX100 achieved 70% viability on day 2 post-editing with 27% HDR on Day 14. EO100, DU100 and DZ100 had 40-50% HDR but only 20-45% viability on day 2 post-editing (FIG. 17A). With respect to viability: ER100<DZ100<DU100<EO100<CM149<CX100. With respect to HDR ER100<CM149<CX100<DZ100<EO100<DU100 (FIG. 17B). High viability and high HDR was preferred and thus CX100 program was the most desirable platform to combine with RNP and ssODN delivery to achieve HDR in long-term HSC (FIG. 17C).

### Example 7—Assessment of ssODN

An assessment of ssODN (pmol titration) with CX100 and DU100 to maximize viability and HDR was performed. Alternative doses of ssODN were assessed in association with the best performing Lonza nucleofection methods in order to find conditions that maximized cell viability at Day 2 and HDR rates. ssODNs were tested at 100 pmol, 50 pmol, and 25 pmol with RNP using Lonza DU100 or CX100 programs.

CX100 led to greater viability than DU100. CX100 at 50 pmol+RNP had a viability of 80% and HDR of 30%. RNP disruption was identical with both methods. Residual NHEJ was higher with RNP+50 pmol of ssODN using CX100 compared to DU100 (FIG. 18).

Examples 4-7 illustrate conditions that maximized HDR while preserving viability using various Lonza nucleofection methods. Overall, SFEM-II media was more desirable than SCGM media as more cells were preserved in the LT-HSC compartment. Lonza CX100 program preserved 80% viability of edited cells while driving an HDR outcome of 30%. Thus, the studies revealed a limited range of conditions most suitable for clinical translation of HDR editing of HBB using ssODN and rAAV6 including: (a) Use of SFEM-II media with low density culturing conditions using 250,000 to 1000,000 cells/ml; (b) Use of cytokines at 100 ng/ml of IL-6, TPO, FLT-3L, SCF in SFEM-II media; (c) Use of alternative Lonza programs with optimal outcome using: (i) ER100<CM149<CX100<DZ100<EO100<DU100 for maximizing HDR; (ii) ER100<DZ100<DU100<EO100<CM149<CX100 for maximizing viability; (iii) Use of RNP at a ratio of 1:1, 1:2.5, 1:5 (using 20 pmol to 40 pmol of Cas9); (iv) Use of ssODN at 10 pmol to 100 pmol concentration for 200,000 cells; (v) Use of rAAV6 at 1-3% culture volume, MOI 2000 to 6000 based on viral titer.

### Example 8—Detection of HDR and Wild Type Outcomes In Vivo and In Vitro

A ddPCR assay was used for measuring both HDR events and unedited (Wild-type) events. An assay was developed as a mutually exclusive assay where either the HDR FAM probe binds or the Wild type HEX probe binds the genomic DNA (gDNA). The FAM and HEX probes were mixed together and allowed to compete for the binding site in the same well. A reference-HEX probe that binds all gDNA within the same amplicon was used as an internal reference and run in parallel. The calculations were the following:

% HDR=(% FAM+)/(Ref HEX+)

% WT=(% HEX+)/(Ref HEX+)

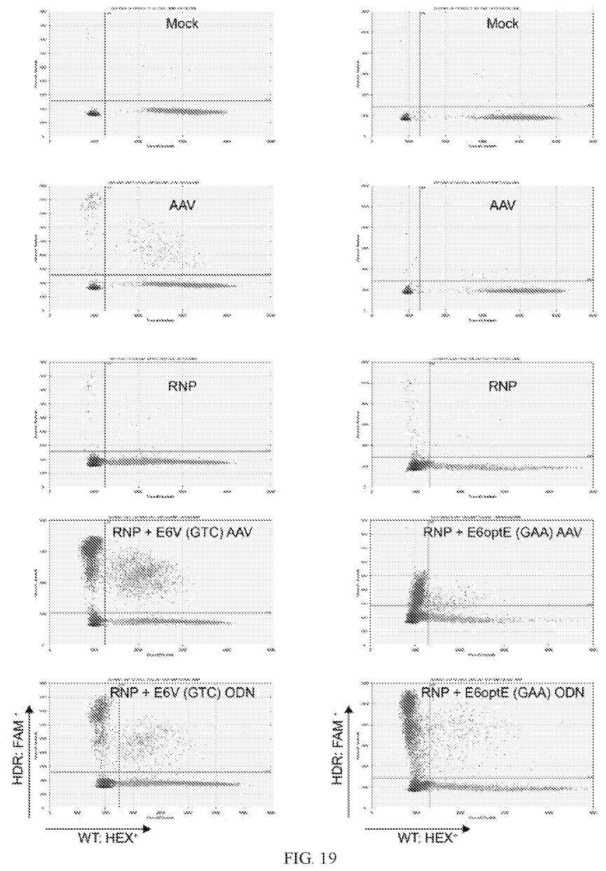

FIG. 19 shows the ddPCR assay results for representative Mock, AAV, RNP, RNP+AAV and RNP+ssODN samples for both the E6V (GTC) change and EoptE (GAA) change. All FAM+ events distinctly represented HDR events and HEX+ events represented wild-type events.

### Example 9—Validation of HDR by ddPCR and NHEJ by ICE

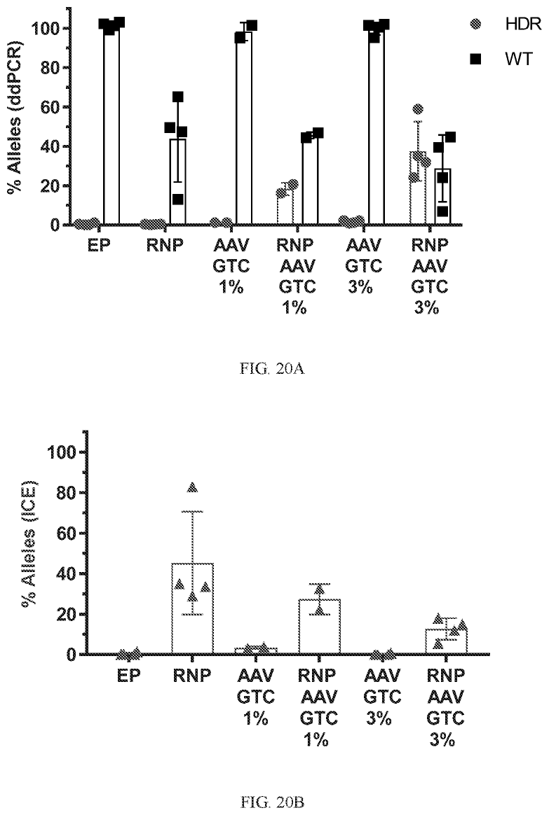

Percent (%) HDR was calculated from ddPCR data (FIG. 20A), and % NHEJ was calculated from ICE algorithm data (FIG. 20B). ICE analysis was used for determining % NHEJ. The ICE algorithm can be used to determine both the knock out rate (NHEJ) as well as knock in rates (HDR). The guide sequence and a donor template sequence were provided to discriminate between indels and HDR distinctly. The analysis delivered sequence traces that were verified to ensure that indels and HDR were identified correctly. The HDR and NHEJ data were combined into one graph to show the total editing rates (Total editing=% NHEJ+% HDR) as well as to demonstrate that with increase in HDR there is a decrease in NHEJ. The HDR and NHEJ data were further validated by MiSeq analysis using the Crispresso algorithm which confirmed the ICE and ddPCR data and demonstrates that with RNP delivery the majority of NHEJ events are primarily deletions.

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific alternatives disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1.-101. (canceled)

102. A system for modifying an HBB gene in a cell, comprising:
a polynucleotide encoding a guide RNA (gRNA); and
a template polynucleotide encoding at least a portion of the HBB gene, or a complement thereof.

103. The system of claim 102, wherein:
the gRNA comprises a nucleic acid having at least 95% identity to the nucleotide sequence of any one of SEQ ID NOs:01-12; and
the at least a portion of the HBB gene comprises exon 1 of the HBB gene.

104. The system of claim 102, further comprising a nucleic acid encoding a nuclease.

105. The system of claim 104, wherein the nuclease is selected from a TALEN nuclease or a Cas nuclease.

106. The system of claim 102, wherein a viral vector comprises the template polynucleotide.

107. The system of claim 106, wherein the viral vector is an adeno-associated viral (AAV) vector.

108. The system of claim 107, wherein the AAV vector is a self-complementary AAV (scAAV) vector.

109. The system of claim 102, wherein the template polynucleotide comprises a single-stranded donor oligonucleotide (ssODN).

110. The system of claim 109, wherein the ssODN comprises a nucleotide sequence having at least 95% identity to the nucleotide sequence of any one of SEQ ID NOs:64-72.

111. The system of claim 102, wherein the HBB gene has at least 95% identity with the nucleotide sequence of SEQ ID NO:37.

112. A method for modifying an HBB gene in a cell, comprising:
(i) providing the system of claim 102;
(ii) introducing the polynucleotide encoding the gRNA into the cell, and
(iii) introducing the template polynucleotide into the cell.

113. The method of claim 112, wherein step (ii) comprises contacting the cell with a ribonucleoprotein (RNP) comprising a Cas9 protein and the polynucleotide encoding the gRNA, wherein the Cas9 protein and the polynucleotide encoding the gRNA have a ratio between 0.1:1 and 1:10.

114. The method of claim 112, wherein a double-strand break is created in exon 1 of the HBB gene.

115. A cell comprising the system of claim 102.

116. The cell of claim 115, wherein the cell is selected from a hematopoietic stem cell, a T cell, a B cell, or a CD34⁺ cell.

117. A pharmaceutical composition comprising the cell of claim 115.

118. A method of treating, inhibiting, or ameliorating a disorder in a subject comprising: administering the cell of claim 115 to the subject in need thereof.

119. The method of claim 118, wherein the cell is administered in combination with a nuclease selected from a Cas nuclease or a TALEN nuclease.

120. The method of claim 118, wherein the disorder comprises sickle cell disease (SCD).

121. The method of claim 120, wherein the SCD comprises a sickle cell mutation comprising an E7V mutation.


SEQUENCE LISTING (81 sequences)

SEQ ID NO: 1 - DNA, 20 bp, Artificial Sequence
  g1 or SCL-1
gtaacggcagacttctcctc

SEQ ID NO: 2 - DNA, 20 bp, Artificial Sequence
  g2 or SCL-2
gtctgccgttactgccctgt

SEQ ID NO: 3 - DNA, 19 bp, Artificial Sequence
  g3 or SCL-3
tctgccgttactgccctgt

SEQ ID NO: 4 - DNA, 20 bp, Artificial Sequence
  g4 or SCL-4
agtctgccgttactgccctg

SEQ ID NO: 5 - DNA, 20 bp, Artificial Sequence
  g5 or SCL-5
aaggtgaacgtggatgaagt

SEQ ID NO: 6 - DNA, 20 bp, Artificial Sequence
  g6 or SCL-6
cttgccccacagggcagtaa

SEQ ID NO: 7 - DNA, 100 bp, Artificial Sequence
  SCL-1 + gRNA scaffold
gtaacggcagacttctcctcgttttagagctagaaatagcaagttaaaataaggctagtccgttatcaacttgaaaaagtggcaccgagtcggtgctttt

SEQ ID NO: 8 - DNA, 100 bp, Artificial Sequence
  SCL-2 + gRNA scaffold
gtctgccgttactgccctgtgttttagagctagaaatagcaagttaaaataaggctagtccgttatcaacttgaaaaagtggcaccgagtcggtgctttt

SEQ ID NO: 9 - DNA, 99 bp, Artificial Sequence
  SCL-3 + gRNA scaffold
tctgccgttactgccctgtgttttagagctagaaatagcaagttaaaataaggctagtccgttatcaacttgaaaaagtggcaccgagtcggtgctttt

SEQ ID NO: 10 - DNA, 100 bp, Artificial Sequence
  SCL-4 + gRNA scaffold
agtctgccgttactgccctggttttagagctagaaatagcaagttaaaataaggctagtccgttatcaacttgaaaaagtggcaccgagtcggtgctttt

SEQ ID NO: 11 - DNA, 100 bp, Artificial Sequence
  SCL-5 + gRNA scaffold
aaggtgaacgtggatgaagtgttttagagctagaaatagcaagttaaaataaggctagtccgttatcaacttgaaaaagtggcaccgagtcggtgctttt

SEQ ID NO: 12 - DNA, 100 bp, Artificial Sequence
  SCL-6 + gRNA scaffold
cttgccccacagggcagtaagttttagagctagaaatagcaagttaaaataaggctagtccgttatcaacttgaaaaagtggcaccgagtcggtgctttt

SEQ ID NO: 13 - DNA, 3 bp, Artificial Sequence
  SCL-1 PAM sequence
agg

SEQ ID NO: 14 - DNA, 3 bp, Artificial Sequence
  SCL-2 PAM sequence
ggg

SEQ ID NO: 15 - DNA, 3 bp, Artificial Sequence
  SCL-3 PAM sequence
ggg

SEQ ID NO: 16 - DNA, 3 bp, Artificial Sequence
  SCL-4 PAM sequence
tgg

SEQ ID NO: 17 - DNA, 3 bp, Artificial Sequence
  SCL-5 PAM sequence
tgg

SEQ ID NO: 18 - DNA, 3 bp, Artificial Sequence
  SCL-6 PAM sequence
cgg

SEQ ID NO: 19 - DNA, 168 bp, Artificial Sequence
  ssODN-V7EGTC
tcagggcagagccatctattgcttacatttgcttctgacacaactgtgttcactagcaacctcaaacagacaccatggtgcatctgactcctgtcgagaagtctgccgttactgccctgtggggcaaggtgaacgtggatgaagttggtggtgaggccctgggcaggt

SEQ ID NO: 20 - DNA, 168 bp, Artificial Sequence
  ssODN-V7EGTG
tcagggcagagccatctattgcttacatttgcttctgacacaactgtgttcactagcaacctcaaacagacaccatggtgcatctgactcctgtggagaagtctgccgttactgccctgtggggcaaggtgaacgtggatgaagttggtggtgaggccctgggcaggt

SEQ ID NO: 21 - DNA, 168 bp, Artificial Sequence
  ssODN CCCGAA
tcagggcagagccatctattgcttacatttgcttctgacacaactgtgttcactagcaacctcaaacagacaccatggtgcatctgactcccgaagagaagtctgccgttactgccctgtggggcaaggtgaacgtggatgaagttggtggtgaggccctgggcaggt

SEQ ID NO: 22 - DNA, 15662 bp, Artificial Sequence
  FrankenpEVL_SCL L Talen
gcgtataatggactattgtgtgctgataaggagaacataagcgcagaacaatatgtatctattccggtgttgtgttcctttgttattctgctattatgttctcttatagtgtgacgaaagcagcataattaatcgccacttgttctttgattgtgttacgatatccagagacttagaaacgggggaaccgggatgagcaaggtaaaaatcggtgagttgatcaacacgcttgtgaatgaggtagaggcaattgatgcctcagaccgcccacaaggcgacaaaacgaagagaattaaagccgcagccgcacggtataagaacgcgttatttaatgataaaagaaagttccgtgggaaaggattgcagaaaagaataaccgcgaatacttttaacgcctatatgagcagggcaagaaagcggtttgatgataaattacatcatagctttgataaaaatattaataaattatcggaaaagtatcctctttacagcgaagaattatcttcatggctttctatgcctacggctaatattcgccagcacatgtcatcgttacaatctaaattgaaagaaataatgccgcttgccgaagagttatcaaatgtaagaataggctctaaaggcagtgatgcaaaaatagcaagactaataaaaaaatatccagattggagttttgctcttagtgatttaaacagtgatgattggaaggagcgccgtgactatctttataagttattccaacaaggctctgcgttgttagaagaactacaccagctcaaggtcaaccatgaggttctgtaccatctgcagctaagccctgcggagcgtacatctatacagcaacgatgggccgatgttctgcgcgagaagaagcgtaatgttgtggttattgactacccaacatacatgcagtctatctatgatattttgaataatcctgcgactttatttagtttaaacactcgttctggaatggcacctttggcctttgctctggctgcggtatcagggcgaagaatgattgagataatgtttcagggtgaatttgccgtttcaggaaagtatacggttaatttctcagggcaagctaaaaaacgctctgaagataaaagcgtaaccagaacgatttatactttatgcgaagcaaaattattcgttgaattattaacagaattgcgttcttgctctgctgcatctgatttcgatgaggttgttaaaggatatggaaaggatgatacaaggtctgagaacggcaggataaatgctattttagcaaaagcatttaacccttgggttaaatcatttttcggcgatgaccgtcgtgtttataaagatagccgcgctatttacgctcgcatcgcttatgagatgttcttccgcgtcgatccacggtggaaaaacgtcgacgaggatgtgttcttcatggagattctcggacacgacgatgagaacacccagctgcactataagcagttcaagctggccaacttctccagaacctggcgacctgaagttggggatgaaaacaccaggctggtggctctgcagaaactggacgatgaaatgccaggctttgccagaggtgacgctggcgtccgtctgcatgaaaccgttaagcagctggtggagcaggacccatcagcaaaaataaccaacagcactctccgggcctttaaatttagcccgacgatgattagccggtacctggagtttgccgctgatgcattggggcagttcgttggcgagaacgggcagtggcagctgaagatagagacacctgcaatcgtcctgcctgatgaagaatccgttgaaaccatcgacgaaccggatgatgagtcccaagacgacgagctggatgaagatgaaattgagctcgacgagggtggcggcgatgaaccaaccgaagaggaagggccagaagaacatcagccaactgctctaaaacccgtcttcaagcctgcaaaaaataacggggacggaacgtacaagatagagtttgaatacgatggaaagcattatgcctggtccggccccgccgatagccctatggccgcaatgcgatccgcatgggaaacgtactacagctaaaagaaaagccaccggtgttaatcggtggcttttttattgaggcctgtccctacccatcccctgcaagggacggaaggattaggcggaaactgcagctgcaactacggacatcgccgtcccgactgcagggacttccccgcgtaaagcggggcttaaattcgggctggccaaccctatttttctgcaatcgctggcgatgttagtttcgtggatagcgtttccagcttttcaatggccagctcaaaatgtgctggcagcaccttctccagttccgtatcaatatcggtgatcggcagctctccacaagacatactccggcgaccgccacgaactacatcgcgcagcagctcccgttcgtagacacgcatgttgcccagagccgtttctgcagccgttaatatccggcgcagctcggcgatgattgccgggagatcatccacggttattgggttcggtgatgggttcctgcaggcgcggcggagagccatccagacgccgctaacccatgcgttacggtactgaaaactttgtgctatgtcgtttatcaggccccgaagttcttctttctgccgccagtccagtggttcaccggcgttcttaggctcaggctcgacaaaagcatactcgccgtttttccggatagctggcagaacctcgttcgtcacccacttgcggaaccgccaggctgtcgtcccctgtttcaccgcgtcgcggcagcggaggattatggtgtagaggccagattccgataccacatttacttccctggccatccgatcaagtttttgtgcctcggttaaaccgagggtcaatttttcatcatgatccagcttacgcaatgcatcagaagggttggctatattcaatgcagcacagatatccagcgccacaaaccacgggtcaccaccgacaagaaccacccgtatagggtggctttcctgaaatgaaaagacggagagagccttcattgcgcctccccggatttcagctgctcagaaagggacagggagcagccgcgagcttcctgcgtgagttcgcgcgcgacctgcagaagttccgcagcttcctgcaaatacagcgtggcctcataactggagatagtgcggtgagcagagcccacaagcgcttcaacctgcagcaggcgttcctcaatcgtcttcagcaggccctgggcgtttaactgaatctggttcatgcgatcacctcgctgaccgggatacgggctgacagaacgaggacaaaacggctggcgaactggcgacgagcttctcgctcggatgatgcagtggtggaaaggcggtggatatgggattttttgtccgtgcggacgacagctgcaaatttgaatttgaacatggtatgcattcctatcttgtatagggtgctaccaccagagttgagaatctctataggggtggtagcccagacagggttctcaacaccggtacaagaagaaaccggcccaaccgaagttggccccatctgagccaccataattcaggtatgcgcagatttaacacacaaaaaaacacgctggcgcgtgttgtgcgcttcttgtcattcggggttgagaggcccggctgcagattttgctgcagcggggtaactctaccgccaaagcagaacgcacgtcaataatttaggtggatattttaccccgtgaccagtcacgtgcacaggtgtttttatagtttgctttactgactgatcagaacctgatcagttattggagtccggtaatcttattgatgaccgcagccaccttagatgttgtctcaaaccccatacggccacgaatgagccactggaacggaatagtcagcaggtacagcggaacgaaccacaaacggttcagacgctgccagaacgtcgcatcacgacgttccatccattcggtattgtcgacgacctggtaagcgtattgtcctggcgtttttgctgcttccgagtagcaatcctcttcaccacaaagaaagttacttatctgcttccagttttcgaacccttcttctttgagccgcttttccagctcattcctccacaaaacaggcacccatcctctgcgataaatcatgattatttgtcctttaaataaggctgtagaactgcaaaatcgctctcgttcacatgctgtacgtagatgcgtagcaaattgccgttccatccctgtaatccaccttctttggaaagatcgtccttgacctcacgaagaactttatccaatagccctgcggcacaagaaattgcctgctctggatcagcaaattcatattgattaataggtgattgccacacaccaaaaacaggaatcatcttttcggctaaacgcctctcctgttctttcttaatctcaagttgtaagcggaccagctcaccatccatcattttttgtagatcatgcgccactattcacccccactggccatcagcaaataaagcttcatactcggacaccggcaggcggcttccacggattgaaaggtcaagccaaccacgtccagatgggtcagccttatccgattcttcccaccgttctgcagctgtagcaaccaggcattctaccgccttcatgtagtcttctgtacggaaccagccgtagttaatgccaccatcagtaactgcccaggccatctttttctcttcggcctcaatagcccggatgcggttatcgcacagctcgcgacagtacttcagctgttcgtaatccagttgcttcaggaactctggtgtcgacgtcatagtggcttcaccttataggcttttagaagcgccctggcttcgtctgtgtggtcttccatgctcttatcgctggcaatgcagcaataaactccctcactatctgagaacccgttcatccgaatgatcgtgaatggaagttcccggccagttttataatcgctatagcttgtcgcgtcgtggctgaccttgaccacataagggtcgtagccctccacgatgacaaggcattcccgttgttttcccattacccctccggttatatcgccacggcttgccgctggcttagaaacgctttcagcagccttatttcgcgtactgatagcaggtccataaattcggtcatgtacagcgaggcgaacgttctcgcgatgctggccactggccacaggcgtaccgcctccatttcggttgctggcaacgcgttctccgcccacgcctccggtaccgccaccgggatagcctccagtgcctggataattactgattgtggggcgtccggaacgtgctctgttttggatcgagggttaccatgtatatctatatttagatccaaatcgcgatccacttcgatggtggttttttccaccttacgtgcgtgaattgataaaccggcctcgcggcgcttctccacgatattcatgaggaactcgaccgagtccgggtcaatggaacgcatcgtggggcgtgcatcgccatctctggcgcgtctggtcttactggatagccccatagactccaggatgcctatgcagaggtctgcaggcgctttcttcttgcctttctctgtgttgaagccgccgatgcgtaaaacgttgtttagcagatcgcgccgttccggcgtgagcaggttatctctggcgcgtttgagggcgtccatgtctgcttcaccttccagggtttttggatcgataccgcagtcgcggaagtactgctgcagcgtcgccgatttgagggtgtagaaaccacgcatgcctatctcaacagcaggggtcgatttcactcggtaatcggttatggccgggaatttagcctggaactctgcgtcggcctgttcccgcgtcatggccgtagtgacgaactgctgccatcttccggcaacgcgataagcgtaggtaaagtgaatcaacgcttcttcacggtcaaggcgacgggcggttatctcatccagctgcatggtttcaaacaggcgcacttttttcaggccgccgtcgaaatagaattttaacgccacctcgtcgacatccagctgcagctccttttcgatgtcccagcggaccagctgggcctgctcatccagggacagggtgcgtttttttatcaactcatcgtgttcggcctggtcaggagtatcgacactcaggtggcgctccataagctgctcaaagaccagttcacgggcttctttacgtaaatccttaccgatgctgtttgcaagcgcgtcggtggccataggcgcgacctgatagccatcatcatgcatgatgcaaatcatgttgctggcataatcatttctggccgatgcctcgagcgcggcggctttaattttgagctgcatgaatgaagagttagccacgccgagtgaaattcggtcaccgtcaaagacaacgtctgtcagcagcccggagtggccagccgtttcgagcaaggcctgcgcgtaggcgcgtttgattttttccggatcggtttcacgtttaccgcgaagcttgtcgaaaccgataatgtattcctgagctgtacggtcgcggcgcagcatctggatggcgtcgctggggaccacttcgccgcagaacatgccgaaatggcggtggaagtgtttctcctcaatcgatacacctgaagatatcgacgggctgtagatgaggccgtcatattttttcaccatcactttaggctggttggtgaaatcgtcgacttccttctcctgtttgtttttctggttaacgcagagaaactttttgtcagggaactgtagtctcagctgcatggtaacgtcttcggcgaacgtcgaactgtcggtggccagcatgattcgttcgccgcgttgcactgcagcgataacctcggtcatgatccgatttttctcggtataaaatacgcggataggcttgttggtttcgcggttgcgaacgtcgaccgggagttcaatcacgtgaatttgcagccaggcaggtaggcccagctcctcgcgtcgcttcatcgccagttcagccaggtcaacaagcagatcgttggcatcggcatccaccataatggcatgctcttcagtacgcgccagcgcgtcgataagcgtgttgaatacgcctaccgggttttccatcgcacgcccggccagaatggcacgcaggccctgtgttgcttcatcgaagccgaagaagtcatgctggcgcatcagcggttgccagcagcctttaagtatggagttgatgcaaatagtcagcttgttggcatatggcgccatttcctgatagccgggatcctgataatgcagaatgtcggctttcgcgcctttcccttcggtcatcatttcatgcaggccgcctatcagggatacgcggtgcgcgacggaaacgccacgcgtggactgcagcatcagtggacgcaggaggcctgtcgatttacccgaccccatcccggcgcggacaataacgatgccctgcagctgtgcggcgtatgtcatcacctcatcggtcatcctggaggtttcaaaccgtttgtaagtgatgtgtgacgggcgaaggttcgggttggtgatgcgttcactgaacgaacgtgatgtttgcgcggcacggcatttgcgattcaaccggcgcgtaatgtgatctttaacggtaccgttataaatttctgcgatacccatatcccgcagcgtgctgctgaaaaggcgcataagttctttcgggctgtttggtaccgggcatgtcagcatgccaatatcaacggcgcgaagcagttctttggcaaaagtgcgtctgttcagacgcgggagagtacgcagcttattcagcgtgatcgacaacagatcggttgcacggctcagatgatttctcgttaactggcgagcgacttccttcagccctctcaggctgtgcaggtcgttaaaatcgctgcattccagctcagggtcatcctcaaaagttgggtaaacacatttgacgccggaaaacttctccatgatgtcgaatccggtgcggaggcctgtgttgccttttccttcagctgaggatttgcggtcgttatcgagagcgcaagtgatttgcgcagccgggtacatgttcaccagctgctcgacaacgtgaatcatgttgttagcggaaaccgcaatgactaccgcgtcaaagcgttttttcgggtcgtttctggtcgccagccagatggatgccccggtggcgaaaccctctgcagtcgcaattttttgcgccccctgcaggtcgccaataacaaagcatgcaccgacgaaatcaccgttagtgatggcgctggtctggaacttgccaccattcagatcgatacgttgccagccaacaatccgcccgtcttttcttccgtccaggtgggacagaggtatcgccatgtaagttgttggtccacggctccatttcgcactgtcgtgactggtcacgcgacgtatatcacaagcgccaaatacgtcacgaattcccttttttaccgcataaggccaggagccatcttcagctggcgaatgttcccaggcgcgatggaaagccaaccatccaagcaggcgttcctgctccatctgattgttttttaaatcattaacgcgttgttgttcagctcggaggcggcgtgcttcagcctggcgctccatgcgtgcacgttcttcttccggctgagcgaccacggtcgcaccattccgttgctgttcacggcgatactccgaaaacaggaatgaaaagccactccaggagccagcgtcatgcgctttttcaacgaagttaacgaaaggataactgatgccatccttgctctgctcaaggcgtgaatagatttccacacggcctttaaggctcttctgcagagcttccggggaggaattattgtaggtggtatagcgctctacaccaccgcgcggattgagctgaatcttatcagcacacgcaggccagttgataccggccatcttcgccagctcagtcagctcatcacgtgccgcgtcaagcagtgaaaacggatcgctgccaaagcgctccgcgtagaattcttgtaaggtcattttttagcctttccatgcgaattagcattttttcgggttgaaaaaatccgcaggagcagccacaataaacgcactatctttctgaaggacgtatctgcgttatcgtggctacttcctgaaaaaggcccgagtttgccgactcggtttttttttcgtcttttttcggctgctacggtctggttcaaccccgacaaagtatagatcggattaaaccagaattatagtcagcaataaaccctgttattgtatcatctaccctcaaccatgaacgatttgatcgtaccgactacttggtgcacaaattgaagatcacttttatcatggataacccgttgagagttagcactatcaaggtagtaatgctgctcgtcataacgggctaatcgttgaattgtgatctcgccgttattatcacaaaccagtacatcctcacccggtacaagcgtaagtgaagaatcgaccaggataacatctcccggctggtagtttcgctgaatctggttcccgaccgtcagtgcgtaaacggtgttccgttgactcacgaacggcaggaatcgctctgtgttggcaggttctccaggctgccagtctctatccggtcctgtctctgtcgtaccaataacaggaacgcggtctggatcagattcagtgccatacagtatccattgcacgggcttacgcaggcattttgccagcgatagcccgatctccagcgacggcatcacgtcgccacgttctaagttttggacgcccggaagagagattcctacagcttctgccacttgcttcagcgtcagtttcagctctaaacggcgtgctttcagtcgttcgcctcgtgttttcatacccttaatcataaatgatctctttatagctggctataatttttataaattatacctagctttaattttcacttattgattataataatccccatgaaacccgaagaacttgtgcgccatttcggcgatgtggaaaaagcagcggttggcgtgggcgtgacacccggcgcagtctatcaatggctgcaagctggggagattccacctctacgacaaagcgatatagaggtccgtaccgcgtacaaattaaagagtgatttcacctctcagcgcatgggtaaggaagggcataacaaggggatcctctagacgcagaaaggcccacccgaaggtgagccagtgtgattacatttgcggcctaactgtggccagtccagttacgctggagtcactagtgcggccgcgacaacttgtctagggcccaatggcccatacacttagtgtaatacgactcactatagggagagcggccgctttttcagcaagattaagccgccaccatggcgccgcggcctcctaagaagaagcggaaagtcgaattcgtggatctgcgaacactgggctatagccagcagcagcaggagaagatcaaacccaaggtgaggtccacagtcgcacagcaccatgaagccctggtgggccacgggttcactcacgctcatattgtcgcactgtctcagcatccagccgctctgggaaccgtggcagtcacataccagcacatcattactgccctgcccgaggctacccatgaagacatcgtgggagtcggcaaacagtggagcggcgcacgggccctggaggctctgctgaccgacgcaggggaactgagaggaccccctctgcagctggatacagggcagctggtgaagattgctaagaggggaggggtgacagcaatggaagccgtccacgcaagcaggaacgcactgacaggggcccccctgaacctgactccggaccaagtggtggctatcgccagcaaccacggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggtggaaaacaggcccttgaaacggtgcagcggctgttgccggtgctgtgccaggaccatggcctgactccggaccaagtggtggctatcgccagcaaccacggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagcaaccacggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctggtcgctctggcttgcctgggaggacgccctgctatggacgctgtgaagaaaggactgccccacgcacccgaactgattagacgggtgaaccggagaatcggcgagagaacatcccatagggtggcaatctctagaactcagctggtcaagagtgaactggaggaaaagaaatcagagctgcgccacaagctgaaatacgtgcctcatgagtatatcgaactgatcgagattgctcgcaattcaacccaggaccggatcctggaaatgaaagtgatggagttctttatgaaagtctacggatatcgggggaaacacctgggagggagcagaaagccagatggggccatctacacagtgggatcccccatcgactatggcgtgattgtcgatactaaagcctacagcggaggctataacctgcctatcggccaggctgacgagatgcagagatacgtggaggaaaaccagacccgcaataagcatattaaccccaatgaatggtggaaagtgtatcctagctccgtcacagagttcaagtttctgttcgtgagcggacactttaagggcaactacaaagcacagctgactaggctgaatcatatcaccaactgcaatggagccgtgctgtctgtcgaggaactgctgatcgggggagagatgattaaggctggcacactgactctggaggaagtgaggcgcaagttcaacaatggggaaatcaacttctaacctgcaggatgataagctagccccgggcgtacggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaacgagaccttagggccattagacttgaagtcaagcggccgcttacaactggaccttgctggtacatagaactgattaactgaccatttaaatcataccaacatggtcaaataaaacgaaaggctcagtcgaaagactgggcctttcgttttaatctgatcggcacgtaagaggttccaactttcaccataatgaaataagatcactaccgggcgtattttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctggctcaggcgcaatcacgaatgaataacggtttggttggtgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaacctaggtgacagaagtcaaaagcctccggtcggaggcttttgactttctgctagatctgtttcaatgcggtgaagggccaggcagctggggattatgtccagacccggccagcatgttggttttatcgcatattcagcgttgtcgcgtttacccaggtaaaatggaagcagtgtatcgtctgcgtgaatgtgcaaatcaggaacgtaaccgtggtacatagatgcagtcccttgcgggtcgttcccttcaacgagtaggacgcggtgcccttgcaaggctaaccattgcgcctggtgtactgcagatgaggttttataaacccctcccttgtgtgacataacggaaagtacaaccgggtttttatcgtcaggtctttggtttgggttaccaaacacactccgcatatggctaatttggtcaattgtgtagccagcgcgacgttctactcggcccctcatctcaaaatcaggagccggtagacgaccagctttttccgcatctctgatagcctgcggtgttacgccgatcaggtctgcaacttctgttataccccagcggcgagtaatacgacgcgcttccgggctgtcatcgccgaactgtgcgatggcaatagcgcgcgtcatttcctgaccgcgattgatacagtctttcagcaaattaattaacgacatcctgtttcctctcaaacatgcccttatctttgtgtttttcatcatactttacgtttttaaagcaaagcaacataaaaaaagcaaagtgacttagaaaacgcaaagttaaggttcaaatcaattttttgatgcgctacagaagctatttagcttcatctaagcgcaacggtattacttacgttggtatatttaaaacctaacttaatgattttaaatgataataaatcataccaattgctatcaaaagttaagcgaacatgctgattttcacgctgtttatacactttgaggcatctctatctcttctgtctctatattgaaacacaatcaaagaacatcaatccatgtgacatcccccactatctaagaacaccataacagaacacaacataggaatgcaacattaatgtatcaataattcggaacatatgcactatatcatatctcaattacggaacatatcagcacacaattgcccattatacgc

SEQ ID NO: 23 - DNA, 15866 bp, Artificial Sequence
  FrankenpEVL_SCL R Talen
gcgtataatggactattgtgtgctgataaggagaacataagcgcagaacaatatgtatctattccggtgttgtgttcctttgttattctgctattatgttctcttatagtgtgacgaaagcagcataattaatcgccacttgttctttgattgtgttacgatatccagagacttagaaacgggggaaccgggatgagcaaggtaaaaatcggtgagttgatcaacacgcttgtgaatgaggtagaggcaattgatgcctcagaccgcccacaaggcgacaaaacgaagagaattaaagccgcagccgcacggtataagaacgcgttatttaatgataaaagaaagttccgtgggaaaggattgcagaaaagaataaccgcgaatacttttaacgcctatatgagcagggcaagaaagcggtttgatgataaattacatcatagctttgataaaaatattaataaattatcggaaaagtatcctctttacagcgaagaattatcttcatggctttctatgcctacggctaatattcgccagcacatgtcatcgttacaatctaaattgaaagaaataatgccgcttgccgaagagttatcaaatgtaagaataggctctaaaggcagtgatgcaaaaatagcaagactaataaaaaaatatccagattggagttttgctcttagtgatttaaacagtgatgattggaaggagcgccgtgactatctttataagttattccaacaaggctctgcgttgttagaagaactacaccagctcaaggtcaaccatgaggttctgtaccatctgcagctaagccctgcggagcgtacatctatacagcaacgatgggccgatgttctgcgcgagaagaagcgtaatgttgtggttattgactacccaacatacatgcagtctatctatgatattttgaataatcctgcgactttatttagtttaaacactcgttctggaatggcacctttggcctttgctctggctgcggtatcagggcgaagaatgattgagataatgtttcagggtgaatttgccgtttcaggaaagtatacggttaatttctcagggcaagctaaaaaacgctctgaagataaaagcgtaaccagaacgatttatactttatgcgaagcaaaattattcgttgaattattaacagaattgcgttcttgctctgctgcatctgatttcgatgaggttgttaaaggatatggaaaggatgatacaaggtctgagaacggcaggataaatgctattttagcaaaagcatttaacccttgggttaaatcatttttcggcgatgaccgtcgtgtttataaagatagccgcgctatttacgctcgcatcgcttatgagatgttcttccgcgtcgatccacggtggaaaaacgtcgacgaggatgtgttcttcatggagattctcggacacgacgatgagaacacccagctgcactataagcagttcaagctggccaacttctccagaacctggcgacctgaagttggggatgaaaacaccaggctggtggctctgcagaaactggacgatgaaatgccaggctttgccagaggtgacgctggcgtccgtctgcatgaaaccgttaagcagctggtggagcaggacccatcagcaaaaataaccaacagcactctccgggcctttaaatttagcccgacgatgattagccggtacctggagtttgccgctgatgcattggggcagttcgttggcgagaacgggcagtggcagctgaagatagagacacctgcaatcgtcctgcctgatgaagaatccgttgaaaccatcgacgaaccggatgatgagtcccaagacgacgagctggatgaagatgaaattgagctcgacgagggtggcggcgatgaaccaaccgaagaggaagggccagaagaacatcagccaactgctctaaaacccgtcttcaagcctgcaaaaaataacggggacggaacgtacaagatagagtttgaatacgatggaaagcattatgcctggtccggccccgccgatagccctatggccgcaatgcgatccgcatgggaaacgtactacagctaaaagaaaagccaccggtgttaatcggtggcttttttattgaggcctgtccctacccatcccctgcaagggacggaaggattaggcggaaactgcagctgcaactacggacatcgccgtcccgactgcagggacttccccgcgtaaagcggggcttaaattcgggctggccaaccctatttttctgcaatcgctggcgatgttagtttcgtggatagcgtttccagcttttcaatggccagctcaaaatgtgctggcagcaccttctccagttccgtatcaatatcggtgatcggcagctctccacaagacatactccggcgaccgccacgaactacatcgcgcagcagctcccgttcgtagacacgcatgttgcccagagccgtttctgcagccgttaatatccggcgcagctcggcgatgattgccgggagatcatccacggttattgggttcggtgatgggttcctgcaggcgcggcggagagccatccagacgccgctaacccatgcgttacggtactgaaaactttgtgctatgtcgtttatcaggccccgaagttcttctttctgccgccagtccagtggttcaccggcgttcttaggctcaggctcgacaaaagcatactcgccgtttttccggatagctggcagaacctcgttcgtcacccacttgcggaaccgccaggctgtcgtcccctgtttcaccgcgtcgcggcagcggaggattatggtgtagaggccagattccgataccacatttacttccctggccatccgatcaagtttttgtgcctcggttaaaccgagggtcaatttttcatcatgatccagcttacgcaatgcatcagaagggttggctatattcaatgcagcacagatatccagcgccacaaaccacgggtcaccaccgacaagaaccacccgtatagggtggctttcctgaaatgaaaagacggagagagccttcattgcgcctccccggatttcagctgctcagaaagggacagggagcagccgcgagcttcctgcgtgagttcgcgcgcgacctgcagaagttccgcagcttcctgcaaatacagcgtggcctcataactggagatagtgcggtgagcagagcccacaagcgcttcaacctgcagcaggcgttcctcaatcgtcttcagcaggccctgggcgtttaactgaatctggttcatgcgatcacctcgctgaccgggatacgggctgacagaacgaggacaaaacggctggcgaactggcgacgagcttctcgctcggatgatgcagtggtggaaaggcggtggatatgggattttttgtccgtgcggacgacagctgcaaatttgaatttgaacatggtatgcattcctatcttgtatagggtgctaccaccagagttgagaatctctataggggtggtagcccagacagggttctcaacaccggtacaagaagaaaccggcccaaccgaagttggccccatctgagccaccataattcaggtatgcgcagatttaacacacaaaaaaacacgctggcgcgtgttgtgcgcttcttgtcattcggggttgagaggcccggctgcagattttgctgcagcggggtaactctaccgccaaagcagaacgcacgtcaataatttaggtggatattttaccccgtgaccagtcacgtgcacaggtgtttttatagtttgctttactgactgatcagaacctgatcagttattggagtccggtaatcttattgatgaccgcagccaccttagatgttgtctcaaaccccatacggccacgaatgagccactggaacggaatagtcagcaggtacagcggaacgaaccacaaacggttcagacgctgccagaacgtcgcatcacgacgttccatccattcggtattgtcgacgacctggtaagcgtattgtcctggcgtttttgctgcttccgagtagcaatcctcttcaccacaaagaaagttacttatctgcttccagttttcgaacccttcttctttgagccgcttttccagctcattcctccacaaaacaggcacccatcctctgcgataaatcatgattatttgtcctttaaataaggctgtagaactgcaaaatcgctctcgttcacatgctgtacgtagatgcgtagcaaattgccgttccatccctgtaatccaccttctttggaaagatcgtccttgacctcacgaagaactttatccaatagccctgcggcacaagaaattgcctgctctggatcagcaaattcatattgattaataggtgattgccacacaccaaaaacaggaatcatcttttcggctaaacgcctctcctgttctttcttaatctcaagttgtaagcggaccagctcaccatccatcattttttgtagatcatgcgccactattcacccccactggccatcagcaaataaagcttcatactcggacaccggcaggcggcttccacggattgaaaggtcaagccaaccacgtccagatgggtcagccttatccgattcttcccaccgttctgcagctgtagcaaccaggcattctaccgccttcatgtagtcttctgtacggaaccagccgtagttaatgccaccatcagtaactgcccaggccatctttttctcttcggcctcaatagcccggatgcggttatcgcacagctcgcgacagtacttcagctgttcgtaatccagttgcttcaggaactctggtgtcgacgtcatagtggcttcaccttataggcttttagaagcgccctggcttcgtctgtgtggtcttccatgctcttatcgctggcaatgcagcaataaactccctcactatctgagaacccgttcatccgaatgatcgtgaatggaagttcccggccagttttataatcgctatagcttgtcgcgtcgtggctgaccttgaccacataagggtcgtagccctccacgatgacaaggcattcccgttgttttcccattacccctccggttatatcgccacggcttgccgctggcttagaaacgctttcagcagccttatttcgcgtactgatagcaggtccataaattcggtcatgtacagcgaggcgaacgttctcgcgatgctggccactggccacaggcgtaccgcctccatttcggttgctggcaacgcgttctccgcccacgcctccggtaccgccaccgggatagcctccagtgcctggataattactgattgtggggcgtccggaacgtgctctgttttggatcgagggttaccatgtatatctatatttagatccaaatcgcgatccacttcgatggtggttttttccaccttacgtgcgtgaattgataaaccggcctcgcggcgcttctccacgatattcatgaggaactcgaccgagtccgggtcaatggaacgcatcgtggggcgtgcatcgccatctctggcgcgtctggtcttactggatagccccatagactccaggatgcctatgcagaggtctgcaggcgctttcttcttgcctttctctgtgttgaagccgccgatgcgtaaaacgttgtttagcagatcgcgccgttccggcgtgagcaggttatctctggcgcgtttgagggcgtccatgtctgcttcaccttccagggtttttggatcgataccgcagtcgcggaagtactgctgcagcgtcgccgatttgagggtgtagaaaccacgcatgcctatctcaacagcaggggtcgatttcactcggtaatcggttatggccgggaatttagcctggaactctgcgtcggcctgttcccgcgtcatggccgtagtgacgaactgctgccatcttccggcaacgcgataagcgtaggtaaagtgaatcaacgcttcttcacggtcaaggcgacgggcggttatctcatccagctgcatggtttcaaacaggcgcacttttttcaggccgccgtcgaaatagaattttaacgccacctcgtcgacatccagctgcagctccttttcgatgtcccagcggaccagctgggcctgctcatccagggacagggtgcgtttttttatcaactcatcgtgttcggcctggtcaggagtatcgacactcaggtggcgctccataagctgctcaaagaccagttcacgggcttctttacgtaaatccttaccgatgctgtttgcaagcgcgtcggtggccataggcgcgacctgatagccatcatcatgcatgatgcaaatcatgttgctggcataatcatttctggccgatgcctcgagcgcggcggctttaattttgagctgcatgaatgaagagttagccacgccgagtgaaattcggtcaccgtcaaagacaacgtctgtcagcagcccggagtggccagccgtttcgagcaaggcctgcgcgtaggcgcgtttgattttttccggatcggtttcacgtttaccgcgaagcttgtcgaaaccgataatgtattcctgagctgtacggtcgcggcgcagcatctggatggcgtcgctggggaccacttcgccgcagaacatgccgaaatggcggtggaagtgtttctcctcaatcgatacacctgaagatatcgacgggctgtagatgaggccgtcatattttttcaccatcactttaggctggttggtgaaatcgtcgacttccttctcctgtttgtttttctggttaacgcagagaaactttttgtcagggaactgtagtctcagctgcatggtaacgtcttcggcgaacgtcgaactgtcggtggccagcatgattcgttcgccgcgttgcactgcagcgataacctcggtcatgatccgatttttctcggtataaaatacgcggataggcttgttggtttcgcggttgcgaacgtcgaccgggagttcaatcacgtgaatttgcagccaggcaggtaggcccagctcctcgcgtcgcttcatcgccagttcagccaggtcaacaagcagatcgttggcatcggcatccaccataatggcatgctcttcagtacgcgccagcgcgtcgataagcgtgttgaatacgcctaccgggttttccatcgcacgcccggccagaatggcacgcaggccctgtgttgcttcatcgaagccgaagaagtcatgctggcgcatcagcggttgccagcagcctttaagtatggagttgatgcaaatagtcagcttgttggcatatggcgccatttcctgatagccgggatcctgataatgcagaatgtcggctttcgcgcctttcccttcggtcatcatttcatgcaggccgcctatcagggatacgcggtgcgcgacggaaacgccacgcgtggactgcagcatcagtggacgcaggaggcctgtcgatttacccgaccccatcccggcgcggacaataacgatgccctgcagctgtgcggcgtatgtcatcacctcatcggtcatcctggaggtttcaaaccgtttgtaagtgatgtgtgacgggcgaaggttcgggttggtgatgcgttcactgaacgaacgtgatgtttgcgcggcacggcatttgcgattcaaccggcgcgtaatgtgatctttaacggtaccgttataaatttctgcgatacccatatcccgcagcgtgctgctgaaaaggcgcataagttctttcgggctgtttggtaccgggcatgtcagcatgccaatatcaacggcgcgaagcagttctttggcaaaagtgcgtctgttcagacgcgggagagtacgcagcttattcagcgtgatcgacaacagatcggttgcacggctcagatgatttctcgttaactggcgagcgacttccttcagccctctcaggctgtgcaggtcgttaaaatcgctgcattccagctcagggtcatcctcaaaagttgggtaaacacatttgacgccggaaaacttctccatgatgtcgaatccggtgcggaggcctgtgttgccttttccttcagctgaggatttgcggtcgttatcgagagcgcaagtgatttgcgcagccgggtacatgttcaccagctgctcgacaacgtgaatcatgttgttagcggaaaccgcaatgactaccgcgtcaaagcgttttttcgggtcgtttctggtcgccagccagatggatgccccggtggcgaaaccctctgcagtcgcaattttttgcgccccctgcaggtcgccaataacaaagcatgcaccgacgaaatcaccgttagtgatggcgctggtctggaacttgccaccattcagatcgatacgttgccagccaacaatccgcccgtcttttcttccgtccaggtgggacagaggtatcgccatgtaagttgttggtccacggctccatttcgcactgtcgtgactggtcacgcgacgtatatcacaagcgccaaatacgtcacgaattcccttttttaccgcataaggccaggagccatcttcagctggcgaatgttcccaggcgcgatggaaagccaaccatccaagcaggcgttcctgctccatctgattgttttttaaatcattaacgcgttgttgttcagctcggaggcggcgtgcttcagcctggcgctccatgcgtgcacgttcttcttccggctgagcgaccacggtcgcaccattccgttgctgttcacggcgatactccgaaaacaggaatgaaaagccactccaggagccagcgtcatgcgctttttcaacgaagttaacgaaaggataactgatgccatccttgctctgctcaaggcgtgaatagatttccacacggcctttaaggctcttctgcagagcttccggggaggaattattgtaggtggtatagcgctctacaccaccgcgcggattgagctgaatcttatcagcacacgcaggccagttgataccggccatcttcgccagctcagtcagctcatcacgtgccgcgtcaagcagtgaaaacggatcgctgccaaagcgctccgcgtagaattcttgtaaggtcattttttagcctttccatgcgaattagcattttttcgggttgaaaaaatccgcaggagcagccacaataaacgcactatctttctgaaggacgtatctgcgttatcgtggctacttcctgaaaaaggcccgagtttgccgactcggtttttttttcgtcttttttcggctgctacggtctggttcaaccccgacaaagtatagatcggattaaaccagaattatagtcagcaataaaccctgttattgtatcatctaccctcaaccatgaacgatttgatcgtaccgactacttggtgcacaaattgaagatcacttttatcatggataacccgttgagagttagcactatcaaggtagtaatgctgctcgtcataacgggctaatcgttgaattgtgatctcgccgttattatcacaaaccagtacatcctcacccggtacaagcgtaagtgaagaatcgaccaggataacatctcccggctggtagtttcgctgaatctggttcccgaccgtcagtgcgtaaacggtgttccgttgactcacgaacggcaggaatcgctctgtgttggcaggttctccaggctgccagtctctatccggtcctgtctctgtcgtaccaataacaggaacgcggtctggatcagattcagtgccatacagtatccattgcacgggcttacgcaggcattttgccagcgatagcccgatctccagcgacggcatcacgtcgccacgttctaagttttggacgcccggaagagagattcctacagcttctgccacttgcttcagcgtcagtttcagctctaaacggcgtgctttcagtcgttcgcctcgtgttttcatacccttaatcataaatgatctctttatagctggctataatttttataaattatacctagctttaattttcacttattgattataataatccccatgaaacccgaagaacttgtgcgccatttcggcgatgtggaaaaagcagcggttggcgtgggcgtgacacccggcgcagtctatcaatggctgcaagctggggagattccacctctacgacaaagcgatatagaggtccgtaccgcgtacaaattaaagagtgatttcacctctcagcgcatgggtaaggaagggcataacaaggggatcctctagacgcagaaaggcccacccgaaggtgagccagtgtgattacatttgcggcctaactgtggccagtccagttacgctggagtcactagtgcggccgcgacaacttgtctagggcccaatggcccatacacttagtgtaatacgactcactatagggagagcggccgctttttcagcaagattaagccgccaccatggcgccgcggcctcctaagaagaagcggaaagtcgaattcgtggatctgcgaacactgggctatagccagcagcagcaggagaagatcaaacccaaggtgaggtccacagtcgcacagcaccatgaagccctggtgggccacgggttcactcacgctcatattgtcgcactgtctcagcatccagccgctctgggaaccgtggcagtcacataccagcacatcattactgccctgcccgaggctacccatgaagacatcgtgggagtcggcaaacagtggagcggcgcacgggccctggaggctctgctgaccgacgcaggggaactgagaggaccccctctgcagctggatacagggcagctggtgaagattgctaagaggggaggggtgacagcaatggaagccgtccacgcaagcaggaacgcactgacaggggcccccctgaacctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaaccacggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggtggaaaacaggcccttgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaaccacggcggcaagcaagcgctcgaaacggtgcagcggctgttgccggtgctgtgccaggaccatggcctgaccccggaccaagtggtggctatcgccagcaaccacggcggcaagcaagcgctcgaaacggtgcagcggctgttgccggtgctgtgccaggaccatggcctgaccccggaccaagtggtggctatcgccagcaaccacggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaaccacggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaagcattgtggcccagctgagccggcctgatccggcgttggccgcgttgaccaacgaccacctggtcgctctggcttgcctgggaggacgccctgctatggacgctgtgaagaaaggactgccccacgcacccgaactgattagacgggtgaaccggagaatcggcgagagaacatcccatagggtggcaatctctagaactcagctggtcaagagtgaactggaggaaaagaaatcagagctgcgccacaagctgaaatacgtgcctcatgagtatatcgaactgatcgagattgctcgcaattcaacccaggaccggatcctggaaatgaaagtgatggagttctttatgaaagtctacggatatcgggggaaacacctgggagggagcagaaagccagatggggccatctacacagtgggatcccccatcgactatggcgtgattgtcgatactaaagcctacagcggaggctataacctgcctatcggccaggctgacgagatgcagagatacgtggaggaaaaccagacccgcaataagcatattaaccccaatgaatggtggaaagtgtatcctagctccgtcacagagttcaagtttctgttcgtgagcggacactttaagggcaactacaaagcacagctgactaggctgaatcatatcaccaactgcaatggagccgtgctgtctgtcgaggaactgctgatcgggggagagatgattaaggctggcacactgactctggaggaagtgaggcgcaagttcaacaatggggaaatcaacttctaacctgcaggatgataagctagccccgggcgtacggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaacgagaccttagggccattagacttgaagtcaagcggccgcttacaactggaccttgctggtacatagaactgattaactgaccatttaaatcataccaacatggtcaaataaaacgaaaggctcagtcgaaagactgggcctttcgttttaatctgatcggcacgtaagaggttccaactttcaccataatgaaataagatcactaccgggcgtattttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctggctcaggcgcaatcacgaatgaataacggtttggttggtgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaacctaggtgacagaagtcaaaagcctccggtcggaggcttttgactttctgctagatctgtttcaatgcggtgaagggccaggcagctggggattatgtccagacccggccagcatgttggttttatcgcatattcagcgttgtcgcgtttacccaggtaaaatggaagcagtgtatcgtctgcgtgaatgtgcaaatcaggaacgtaaccgtggtacatagatgcagtcccttgcgggtcgttcccttcaacgagtaggacgcggtgcccttgcaaggctaaccattgcgcctggtgtactgcagatgaggttttataaacccctcccttgtgtgacataacggaaagtacaaccgggtttttatcgtcaggtctttggtttgggttaccaaacacactccgcatatggctaatttggtcaattgtgtagccagcgcgacgttctactcggcccctcatctcaaaatcaggagccggtagacgaccagctttttccgcatctctgatagcctgcggtgttacgccgatcaggtctgcaacttctgttataccccagcggcgagtaatacgacgcgcttccgggctgtcatcgccgaactgtgcgatggcaatagcgcgcgtcatttcctgaccgcgattgatacagtctttcagcaaattaattaacgacatcctgtttcctctcaaacatgcccttatctttgtgtttttcatcatactttacgtttttaaagcaaagcaacataaaaaaagcaaagtgacttagaaaacgcaaagttaaggttcaaatcaattttttgatgcgctacagaagctatttagcttcatctaagcgcaacggtattacttacgttggtatatttaaaacctaacttaatgattttaaatgataataaatcataccaattgctatcaaaagttaagcgaacatgctgattttcacgctgtttatacactttgaggcatctctatctcttctgtctctatattgaaacacaatcaaagaacatcaatccatgtgacatcccccactatctaagaacaccataacagaacacaacataggaatgcaacattaatgtatcaataattcggaacatatgcactatatcatatctcaattacggaacatatcagcacacaattgcccattatacgc

SEQ ID NO: 24 - DNA, 7365 bp, Artificial Sequence
  template 1242
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacacgcgtagatctagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccaggtgagttaaacccatgagagagaataacagaactgcgagtgatgggccagttaagcgtagatggctaattagttcagacaaatgtaaaatgccaacaccgtctgtaaagaaacctaactgatcctcttcctttgtcctgtcttcttcacaggccgccaccatggtccatcttacaccggaggagaagtccgctgtaacggcactgtgggggaaagttaatgtcgatgaagtcggcggtgaagcactcggcaggttgctggtagtgtacccgtggacacaacgattctttgaaagtttcggggacctgtccacacccgatgctgtgatgggtaatccaaaagtaaaagcacacggcaagaaagtcctcggcgcgtttagtgatggtctggcccatttggataacttgaagggtacattcgcgcagctttccgaactccactgtgacaagttgcacgtagatccagaaaacttccggcttctgggcaatgtgcttgtatgcgttctggctcaccattttgggaaggagtttaccccacccgtgcaagcggcttaccaaaaagtggtcgcaggagtggctaatgcccttgcacataaatatcactaaggtaccgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgcgggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggcggccgcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagctactaacttcagcctgctgaagcaggctggagacgtggaggagaaccctggacctacctgcaggcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttgtcgacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 25 - DNA, 8087 bp, Artificial Sequence
  template 1243
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacacgcgtagatctagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccaggtgagttaaacccatgagagagaataacagaactgcgagtgatgggccagttaagcgtagatggctaattagttcagacaaatgtaaaatgccaacaccgtctgtaaagaaacctaactgatcctcttcctttgtcctgtcttcttcacaggccgccaccatggtccatcttacaccggaggagaagtccgctgtaacggcactgtgggggaaagttaatgtcgatgaagtcggcggtgaagcactcggcaggttgctggtagtgtacccgtggacacaacgattctttgaaagtttcggggacctgtccacacccgatgctgtgatgggtaatccaaaagtaaaagcacacggcaagaaagtcctcggcgcgtttagtgatggtctggcccatttggataacttgaagggtacattcgcgcagctttccgaactccactgtgacaagttgcacgtagatccagaaaacttccggcttctgggcaatgtgcttgtatgcgttctggctcaccattttgggaaggagtttaccccacccgtgcaagcggcttaccaaaaagtggtcgcaggagtggctaatgcccttgcacataaatatcactaaggtaccgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttaattaacgagatcgagaccatcctggctaacacagtgaaaccccgtctctactaaaaaaatacaaaaaattagccgggcttggtggcgggtgcctgtagtcccagctactatggaggctgaggcgggagaatggcgtgaacgcggggggcggagcttgcagtgagcagagatcaggggccactgcactccagcctgggcgacagagagagactctgtctcaaaaaaaagaaaaaaaaaatttagtagactagctaaaaaaatccagagatagttattgatgcatatgtaaaagtcttccaatatttacaagtacaatgaaaaaaaaataaccttgaattaagtgtagaactcattgacaatgtttcaaaggatgtgagggataaactaaaatttgggcagtacatgctgttcctgtgtacttggaacagagggagaaaatctgggctggaaatattgttataggagttagcacatgaaggtgacaactaaattatttggagtagatggagtcaccagcacatgtgaatagttttagaatgaaatgacccaagatagaactttggagagcccccaaatttaaataaaatcagtataagagaagaggaagaaaccaaatggtatactagtctaaattgtttcttagtgacaaaagaataacctgaatattagattagctgcctatatgctctctgaatcaatttcattcaacatgcaacagtccgcgggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggcggccgcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagctactaacttcagcctgctgaagcaggctggagacgtggaggagaaccctggacctacctgcaggcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttgtcgacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 26 - DNA, 7712 bp, Artificial Sequence
  template 1244
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacacgcgtagatctagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccaggtgagttaaacccatgagagagaataacagaactgcgagtgatgggccagttaagcgtagatggctaattagttcagacaaatgtaaaatgccaacaccgtctgtaaagaaacctaactgatcctcttcctttgtcctgtcttcttcacaggccgccaccatggtccatcttacaccggaggagaagtccgctgtaacggcactgtgggggaaagttaatgtcgatgaagtcggcggtgaagcactcggcaggttgctggtagtgtacccgtggacacaacgattctttgaaagtttcggggacctgtccacacccgatgctgtgatgggtaatccaaaagtaaaagcacacggcaagaaagtcctcggcgcgtttagtgatggtctggcccatttggataacttgaagggtacattcgcgcagctttccgaactccactgtgacaagttgcacgtagatccagaaaacttccggcttctgggcaatgtgcttgtatgcgttctggctcaccattttgggaaggagtttaccccacccgtgcaagcggcttaccaaaaagtggtcgcaggagtggctaatgcccttgcacataaatatcactaaggtaccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcttaattaacgagatcgagaccatcctggctaacacagtgaaaccccgtctctactaaaaaaatacaaaaaattagccgggcttggtggcgggtgcctgtagtcccagctactatggaggctgaggcgggagaatggcgtgaacgcggggggcggagcttgcagtgagcagagatcaggggccactgcactccagcctgggcgacagagagagactctgtctcaaaaaaaagaaaaaaaaaatttagtagactagctaaaaaaatccagagatagttattgatgcatatgtaaaagtcttccaatatttacaagtacaatgaaaaaaaaataaccttgaattaagtgtagaactcattgacaatgtttcaaaggatgtgagggataaactaaaatttgggcagtacatgctgttcctgtgtacttggaacagagggagaaaatctgggctggaaatattgttataggagttagcacatgaaggtgacaactaaattatttggagtagatggagtcaccagcacatgtgaatagttttagaatgaaatgacccaagatagaactttggagagcccccaaatttaaataaaatcagtataagagaagaggaagaaaccaaatggtatactagtctaaattgtttcttagtgacaaaagaataacctgaatattagattagctgcctatatgctctctgaatcaatttcattcaacatgcaacagtccgcgggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggcggccgcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagctactaacttcagcctgctgaagcaggctggagacgtggaggagaaccctggacctacctgcaggcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttgtcgacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 27 - DNA, 7925 bp, Artificial Sequence
  template 1245
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacacgcgtagatctagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtccatcttacaccggaggagaagtccgctgtaacggcactgtgggggaaagttaatgtcgatgaagtcggcggtgaagcactcggcaggttgctggtagtgtacccgtggacacaacgattctttgaaagtttcggggacctgtccacacccgatgctgtgatgggtaatccaaaagtaaaagcacacggcaagaaagtcctcggcgcgtttagtgatggtctggcccatttggataacttgaagggtacattcgcgcagctttccgaactccactgtgacaagttgcacgtagatccagaaaacttccggcttctgggcaatgtgcttgtatgcgttctggctcaccattttgggaaggagtttaccccacccgtgcaagcggcttaccaaaaagtggtcgcaggagtggctaatgcccttgcacataaatatcactaaggtaccgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttaattaacgagatcgagaccatcctggctaacacagtgaaaccccgtctctactaaaaaaatacaaaaaattagccgggcttggtggcgggtgcctgtagtcccagctactatggaggctgaggcgggagaatggcgtgaacgcggggggcggagcttgcagtgagcagagatcaggggccactgcactccagcctgggcgacagagagagactctgtctcaaaaaaaagaaaaaaaaaatttagtagactagctaaaaaaatccagagatagttattgatgcatatgtaaaagtcttccaatatttacaagtacaatgaaaaaaaaataaccttgaattaagtgtagaactcattgacaatgtttcaaaggatgtgagggataaactaaaatttgggcagtacatgctgttcctgtgtacttggaacagagggagaaaatctgggctggaaatattgttataggagttagcacatgaaggtgacaactaaattatttggagtagatggagtcaccagcacatgtgaatagttttagaatgaaatgacccaagatagaactttggagagcccccaaatttaaataaaatcagtataagagaagaggaagaaaccaaatggtatactagtctaaattgtttcttagtgacaaaagaataacctgaatattagattagctgcctatatgctctctgaatcaatttcattcaacatgcaacagtccgcgggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggcggccgcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagctactaacttcagcctgctgaagcaggctggagacgtggaggagaaccctggacctacctgcaggcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttgtcgacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 28 - DNA, 5216 bp, Artificial Sequence
  template 1246
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacacgcgtagatctatatacacacatatatatatatattttttcttttcttaccagaaggttttaatccaaataaggagaagatatgcttagaaccgaggtagagttttcatccattctgtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtccatcttacaccggtggagaaatccgctgtaacggcactttgggggaaagttaatgtcgatgaggtcggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttgtcgacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 29 - DNA, 4816 bp, Artificial Sequence
  template 1247
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacacgcgtagatctagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtccatcttacaccggtggagaaatccgctgtaacggcactttgggggaaagttaatgtcgatgaggtcggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttgtcgacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 30 - DNA, 4416 bp, Artificial Sequence
  template 1248
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacacgcgtagatcttgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtccatcttacaccggtggagaaatccgctgtaacggcactttgggggaaagttaatgtcgatgaggtcggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttgggggtcgacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 31 - DNA, 4116 bp, Artificial Sequence
  template 1249
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacacgcgtagatctacatttgcttctgacacaactgtgttcactagcaacctcaaacagacaccatggtccatcttacaccggtggagaaatccgctgtaacggcactttgggggaaagttaatgtcgatgaggtcggtggtgaggccctgggcaggttggtatcaaggttacaagacaggtttaagtcgacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 32 - DNA, 7995 bp, Artificial Sequence
  template 1289
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacacgcgtagatctagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactcctgaggagaagtccgctgtaacggcactgtgggggaaagttaatgtcgatgaagtcggcggtgaagcactcggcaggttgctggtagtgtacccgtggacacaacgattctttgaaagtttcggggacctgtccacacccgatgctgtgatgggtaatccaaaagtaaaagcacacggcaagaaagtcctcggcgcgtttagtgatggtctggcccatttggataacttgaagggtacattcgcgcagctttccgaactccactgtgacaagttgcacgtagatccagaaaacttccggcttctgggcaatgtgcttgtatgcgttctggctcaccattttgggaaggagtttaccccacccgtgcaagcggcttaccaaaaagtggtcgcaggagtggctaatgcccttgcacataaatatcactaaggtaccgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttaattaacgagatcgagaccatcctggctaacacagtgaaaccccgtctctactaaaaaaatacaaaaaattagccgggcttggtggcgggtgcctgtagtcccagctactatggaggctgaggcgggagaatggcgtgaacgcggggggcggagcttgcagtgagcagagatcaggggccactgcactccagcctgggcgacagagagagactctgtctcaaaaaaaagaaaaaaaaaatttagtagactagctaaaaaaatccagagatagttattgatgcatatgtaaaagtcttccaatatttacaagtacaatgaaaaaaaaataaccttgaattaagtgtagaactcattgacaatgtttcaaaggatgtgagggataaactaaaatttgggcagtacatgctgttcctgtgtacttggaacagagggagaaaatctgggctggaaatattgttataggagttagcacatgaaggtgacaactaaattatttggagtagatggagtcaccagcacatgtgaatagttttagaatgaaatgacccaagatagaactttggagagcccccaaatttaaataaaatcagtataagagaagaggaagaaaccaaatggtatactagtctaaattgtttcttagtgacaaaagaataacctgaatattagattagctgcctatatgctctctgaatcaatttcattcaacatgcaacagtccgcgggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggcggccgcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagctttatttgtgaaatttgtgatgctattgctttatttgtaaccattataagctgcaataaacaagttaacaacaacaattgcattcattttatgtttcaggttcagggggagatgtgggaggttttttaaagccctgcaggaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagcgtcgacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 33 - DNA, 7995 bp, Artificial Sequence
  template 1290
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacacgcgtagatctagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactcctgaggagaagtccgctgtaacggcactgtgggggaaagttaatgtcgatgaagtcggcggtgaagcactcggcaggttgctggtagtgtacccgtggacacaacgattctttgaaagtttcggggacctgtccacacccgatgctgtgatgggtaatccaaaagtaaaagcacacggcaagaaagtcctcggcgcgtttagtgatggtctggcccatttggataacttgaagggtacattcgcgcagctttccgaactccactgtgacaagttgcacgtagatccagaaaacttccggcttctgggcaatgtgcttgtatgcgttctggctcaccattttgggaaggagtttaccccacccgtgcaagcggcttaccaaaaagtggtcgcaggagtggctaatgcccttgcacataaatatcactaaggtaccgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttaattaacgagatcgagaccatcctggctaacacagtgaaaccccgtctctactaaaaaaatacaaaaaattagccgggcttggtggcgggtgcctgtagtcccagctactatggaggctgaggcgggagaatggcgtgaacgcggggggcggagcttgcagtgagcagagatcaggggccactgcactccagcctgggcgacagagagagactctgtctcaaaaaaaagaaaaaaaaaatttagtagactagctaaaaaaatccagagatagttattgatgcatatgtaaaagtcttccaatatttacaagtacaatgaaaaaaaaataaccttgaattaagtgtagaactcattgacaatgtttcaaaggatgtgagggataaactaaaatttgggcagtacatgctgttcctgtgtacttggaacagagggagaaaatctgggctggaaatattgttataggagttagcacatgaaggtgacaactaaattatttggagtagatggagtcaccagcacatgtgaatagttttagaatgaaatgacccaagatagaactttggagagcccccaaatttaaataaaatcagtataagagaagaggaagaaaccaaatggtatactagtctaaattgtttcttagtgacaaaagaataacctgaatattagattagctgcctatatgctctctgaatcaatttcattcaacatgcaacagtccgcgggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggcggccgcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagctttatttgtgaaatttgtgatgctattgctttatttgtaaccattataagctgcaataaacaagttaacaacaacaattgcattcattttatgtttcaggttcagggggagatgtgggaggttttttaaagccctgcaggaggagaagtctgaggttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagcgtcgacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 34 - DNA, 8036 bp, Artificial Sequence
  template 1314
cagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgattcaaacttccgcagaacactttatttcacatatacatgcctcttatatcagggatgtgaaacagggtcttgaaaactgtctaaatctaaaacaatgctaatgcaggtttaaatttaataaaataaaatccaaaatctaacagccaagtcaaatctgcatgttttaacatttaaaatattttaaagacgtcttttcccaggattcaacatgtgaaatcttttctcagggatacacgtgtgcctagatcctcattgctttagttttttacagaggaatgaatataaaaagaaaatacttaaattttatccctcttacctctataatcatacataggcataattttttaacctaggctccagatagccatagaagaaccaaacactttctgcgtgtgtgagaataatcagagtgagattttttcacaagtacctgatgagggttgagacaggtagaaaaagtgagagatctctatttatttagcaataatagagaaagcatttaagagaataaagcaatggaaataagaaatttgtaaatttccttctgataactagaaatagaggatccagtttcttttggttaacctaaattttatttcattttattgttttattttattttattttattttattttgtgtaatcgtagtttcagagtgttagagctgaaaggaagaagtaggagaaacatgcaaagtaaaagtataacactttccttactaaaccgacatgggtttccaggtaggggcaggattcaggatgactgacagggcccttagggaacactgagaccctacgctgacctcataaatgcttgctacctttgctgttttaattacatcttttaatagcaggaagcagaactctgcacttcaaaagtttttcctcacctgaggagttaatttagtacaaggggaaaaagtacagggggatgggagaaaggcgatcacgttgggaagctatagagaaagaagagtaaattttagtaaaggaggtttaaacaaacaaaatataaagagaaataggaacttgaatcaaggaaatgattttaaaacgcagtattcttagtggactagaggaaaaaaataatctgagccaagtagaagaccttttcccctcctacccctactttctaagtcacagaggctttttgttcccccagacactcttgcagattagtccaggcagaaacagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtatatatacacacatatatatatatattttttcttttcttaccagaaggttttaatccaaataaggagaagatatgcttagaaccgaggtagagttttcatccattctgtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactcctgtcgagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcatatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacaggggtcatggtttgactgtcctgtgagcccttcttccctgatcggatcccctgcaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 35 - DNA, 8207 bp, Artificial Sequence
  template 1321
cagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgtagatctagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtatatatacacacatatatatatatattttttcttttcttaccagaaggttttaatccaaataaggagaagatatgcttagaaccgaggtagagttttcatccattctgtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcatctgactccttaagctttaaaaaacctcccacatctccccctgaacctgaaacataaaatgaatgcaattgttgttgttaacttgtttattgcagcttataatggttacaaataaagcaatagcatcacaaatttcacaaataaagcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcggcgcggccgcgatctgacggttcactaaacgagctctgcttatatagagctcggggagcagaagcgcgcgaacagaagcgagaagcgaactgattggttagttcaaataaggcacagggtcatttcaggtccttggggcaccctggaaacatctgatggttctctagaaactgctgagggcgggaccgcatctggggaccatctgttcttggccctgagccggggcaggaactgcttaccacagatatcctgtttggcccatattctgctgttccaactgttcttggccctgagccggggcaggaactgcttaccacagatatcctgtttggcccatattctcctgtttctctgttcccgcggcgagatcgagaccatcctggctaacacagtgaaaccccgtctctactaaaaaaatacaaaaaattagccgggcttggtggcgggtgcctgtagtcccagctactatggaggctgaggcgggagaatggcgtgaacgcggggggcggagcttgcagtgagcagagatcaggggccactgcactccagcctgggcgacagagagagactctgtctcaaaaaaaagaaaaaaaaaatttagtagactagctaaaaaaatccagagatagttattgatgcatatgtaaaagtcttccaatatttacaagtacaatgaaaaaaaaataaccttgaattaagtgtagaactcattgacaatgtttcaaaggatgtgagggataaactaaaatttgggcagtacatgctgttcctgtgtacttggaacagagggagaaaatctgggctggaaatattgttataggagttagcacatgaaggtgacaactaaattatttggagtagatggagtcaccagcacatgtgaatagttttagaatgaaatgacccaagatagaactttggagagcccccaaatttaaataaaatcagtataagagaagaggaagaaaccaaatggtatactagtctaaattgtttcttagtgacaaaagaataacctgaatattagattagctgcctatatgctctctgaatcaatttcattcaacatgcaacagttctggaacctatcagggaccacagtcagccaggcaagcacatctgcccaagccaagggtggaggcatgcagctgtgggggtctgtgaaaacacttgagggagcagataactgggccaaccatgactcagtgcttctggaggccaacaggactgctgagtcatcctgtgggggtggaggtgggacaagggaaaggggtgaatggtactgctgattacaacctctggtgctgcctccccctcctgtttatctgagagaggcctcactggagctagagacaagaaggtaaaaaacggctgacaaaagaagtcctggtatcctctatgatgggagaaggaaactagctaaagggaagaataaattagagaaaaactggaatgactgaatcggaacaaggcaaaggctataaaaaaaattagcagtatcctcttgggggccccttccccacactatctcaatgcaaatatctgtctgaaacggtccctggctaaactccacccatgggttggccagccttgccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatggtccatcttacaccggtggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatcgtcgacaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 36 - DNA, 8009 bp, Artificial Sequence
  template 1322
cagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgtagatctagttagatgtccccagttaacctcctatttgacaccactgattaccccattgatagtcacactttgggttgtaagtgactttttatttatttgtatttttgactgcattaagaggtctctagttttttatctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtatatatacacacatatatatatatattttttcttttcttaccagaaggttttaatccaaataaggagaagatatgcttagaaccgaggtagagttttcatccattctgtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtgctttaaaaaacctcccacatctccccctgaacctgaaacataaaatgaatgcaattgttgttgttaacttgtttattgcagcttataatggttacaaataaagcaatagcatcacaaatttcacaaataaagcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcggcgcggccgcgatctgacggttcactaaacgagctctgcttatatagagctcggggagcagaagcgcgcgaacagaagcgagaagcgaactgattggttagttcaaataaggcacagggtcatttcaggtccttggggcaccctggaaacatctgatggttctctagaaactgctgagggcgggaccgcatctggggaccatctgttcttggccctgagccggggcaggaactgcttaccacagatatcctgtttggcccatattctgctgttccaactgttcttggccctgagccggggcaggaactgcttaccacagatatcctgtttggcccatattctcctgtttctctgttcccgcggcgagatcgagaccatcctggctaacacagtgaaaccccgtctctactaaaaaaatacaaaaaattagccgggcttggtggcgggtgcctgtagtcccagctactatggaggctgaggcgggagaatggcgtgaacgcggggggcggagcttgcagtgagcagagatcaggggccactgcactccagcctgggcgacagagagagactctgtctcaaaaaaaagaaaaaaaaaatttagtagactagctaaaaaaatccagagatagttattgatgcatatgtaaaagtcttccaatatttacaagtacaatgaaaaaaaaataaccttgaattaagtgtagaactcattgacaatgtttcaaaggatgtgagggataaactaaaatttgggcagtacatgctgttcctgtgtacttggaacagagggagaaaatctgggctggaaatattgttataggagttagcacatgaaggtgacaactaaattatttggagtagatggagtcaccagcacatgtgaatagttttagaatgaaatgacccaagatagaactttggagagcccccaaatttaaataaaatcagtataagagaagaggaagaaaccaaatggtatactagtctaaattgtttcttagtgacaaaagaataacctgaatattagattagctgcctatatgctctctgaatcaatttcattcaacatgcaacagttctggaacctatcagggaccacagtcagccaggcaagcacatctgcccaagccaagggtggaggcatgcagctgtgggggtctgtgaaaacacttgagggagcagataactgggccaaccatgactcagtgcttctggaggccaacaggactgctgagtcatcctgtgggggtggaggtgggacaagggaaaggggtgaatggtactgctgattacaacctctggtgctgcctccccctcctgtttatctgagagaggcctcactggagctagagacaagaaggtaaaaaacggctgacaaaagaagtcctggtatcctctatgatgggagaaggaaactagctaaagggaagaataaattagagaaaaactggaatgactgaatcggaacaaggcaaaggctataaaaaaaattagcagtatcctcttgggggccccttccccacactatctcaatgcaaatatctgtctgaaacggtccctggctaaactccacccatgggttggccagccttgccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatggtccatcttacaccggtggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatcgtcgacaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 37 - DNA, 1606 bp, Artificial Sequence
  gene sequence of HBB hemoglobin subunit beta [Homo sapiens (human)] Gene ID 3043 on NCBI (updated on 21-Apr-2018), located at 11p15.4 of the human genome. The sequence starts at the beginning of Exon 1. The sequence is identified on the NCBI database as NC_000011.10c5227071-5225466 Homo sapiens chromosome 11, GRCh38.p12 Primary Assembly
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgc

SEQ ID NO: 38 - DNA, 36 bp, Artificial Sequence
  primer
gtcgagaagtctgcagtcactgctctatgggggaaa

SEQ ID NO: 39 - DNA, 20 bp, Artificial Sequence
  HBB-1250
aggctttttgttcccccaga

SEQ ID NO: 40 - DNA, 20 bp, Artificial Sequence
  HBB-1250
agccttcaccttagggttgc

SEQ ID NO: 41 - DNA, 20 bp, Artificial Sequence
  SCL-386
gggttggccaatctactccc

SEQ ID NO: 42 - DNA, 20 bp, Artificial Sequence
  SCL-386
cctctgggtccaagggtaga

SEQ ID NO: 43 - DNA, 19 bp, Artificial Sequence
  ddPCR
cataaaagtcagggcagag

SEQ ID NO: 44 - DNA, 20 bp, Artificial Sequence
  ddPCR
gtctccttaaacctgtcttg

SEQ ID NO: 45 - DNA, 22 bp, Artificial Sequence
  LINC01206
caaaaagcaaaatttggggata

SEQ ID NO: 46 - DNA, 20 bp, Artificial Sequence
  LINC01206
cttttagcccagtgccagac

SEQ ID NO: 47 - DNA, 20 bp, Artificial Sequence
  MIR7974
atcagcccctctttctggat

SEQ ID NO: 48 - DNA, 20 bp, Artificial Sequence
  MIR7974
agtgcagtggtgccatcata

SEQ ID NO: 49 - DNA, 20 bp, Artificial Sequence
  HBD
cagatccccaaaggactcaa

SEQ ID NO: 50 - DNA, 20 bp, Artificial Sequence
  HBD
gcggtggggagatatgtaga

SEQ ID NO: 51 - DNA, 20 bp, Artificial Sequence
  TULP4
cacgccaggatgtaagctct

SEQ ID NO: 52 - DNA, 20 bp, Artificial Sequence
  TULP4
tctgaggcaaaagtgcaaga

SEQ ID NO: 53 - DNA, 20 bp, Artificial Sequence
  DENND3
gggggtttctatccctcact

SEQ ID NO: 54 - DNA, 20 bp, Artificial Sequence
  DENND3
caagagggtcaggttgagga

SEQ ID NO: 55 - DNA, 53 bp, Artificial Sequence
  HBB-Miseq (with adapter)
tcgtcggcagcgtcagatgtgtataagagacaggggttggccaatctactccc

SEQ ID NO: 56 - DNA, 54 bp, Artificial Sequence
  HBB-Miseq (with adapter)
gtctcgtgggctcggagatgtgtataagagacagcctctgggtccaagggtaga

SEQ ID NO: 57 - DNA, 55 bp, Artificial Sequence
  HBD-Miseq (with adapter)
tcgtcggcagcgtcagatgtgtataagagacagcacaaactaatgaaaccctgct

SEQ ID NO: 58 - DNA, 55 bp, Artificial Sequence
  HBD-Miseq (with adapter)
gtctcgtgggctcggagatgtgtataagagacagtctacacatgcccagtttcca

SEQ ID NO: 59 - DNA, 19 bp, Artificial Sequence
  GTC HDR FAM
ctcctgtcgagaagtctgc

SEQ ID NO: 60 - DNA, 19 bp, Artificial Sequence
  GAA HDR FAM
ctcccgaagagaagtctgc

SEQ ID NO: 61 - DNA, 19 bp, Artificial Sequence
  GTG HDR FAM
ctcctgtggagaagtctgc

SEQ ID NO: 62 - DNA, 18 bp, Artificial Sequence
  GAG WT HEX
tgactcctgtcgagaagt

SEQ ID NO: 63 - DNA, 27 bp, Artificial Sequence
  REF HEX
gttcactagcaacctcaaacagacacc

SEQ ID NO: 64 - DNA, 59 bp, Artificial Sequence
  E6V GAG > GTC
tcagggcagagccatctattgcttacatttgcttctgacacaactgtgttcactagcaa

SEQ ID NO: 65 - DNA, 59 bp, Artificial Sequence
  E6V GAG > GTC
cctcaaacagacaccatggtgcatctgactcctgtcgagaagtctgccgttactgccct

SEQ ID NO: 66 - DNA, 50 bp, Artificial Sequence
  E6V GAG > GTC
gtggggcaaggtgaacgtggatgaagttggtggtgaggccctgggcaggt

SEQ ID NO: 67 - DNA, 59 bp, Artificial Sequence; identical to SEQ ID NO: 64
  E6V GAG > CCC GAA

SEQ ID NO: 68 - DNA, 59 bp, Artificial Sequence
  E6V GAG > CCC GAA
cctcaaacagacaccatggtgcatctgactcccgaagagaagtctgccgttactgccct

SEQ ID NO: 69 - DNA, 50 bp, Artificial Sequence; identical to SEQ ID NO: 66
  E6V GAG > CCC GAA

SEQ ID NO: 70 - DNA, 59 bp, Artificial Sequence; identical to SEQ ID NO: 64
  E6V GAG > GTG

SEQ ID NO: 71 - DNA, 59 bp, Artificial Sequence
  E6V GAG > GTG
cctcaaacagacaccatggtgcatctgactcctgtggagaagtctgccgttactgccct

SEQ ID NO: 72 - DNA, 50 bp, Artificial Sequence; identical to SEQ ID NO: 66
  E6V GAG > GTG

SEQ ID NO: 73 - DNA, 75 bp, Artificial Sequence
  primer
caacctcaaacagacaccatggtgcatctgactcctgaggagaagtctgccgttactgccctgtggggcaaggtg

SEQ ID NO: 74 - DNA, 72 bp, Artificial Sequence
  primer
caacctcaaacagacaccatggtgcacctgactcctgagaagtctgccgttactgccctgtggggcaaggtg

SEQ ID NO: 75 - DNA, 70 bp, Artificial Sequence
  primer
caacctcaaacagacaccatggtgcacctgactcctgaagtctgccgttactgccctgtggggcaaggtg

SEQ ID NO: 76 - DNA, 63 bp, Artificial Sequence
  primer
caacctcaaacagacaccatggtgcacctgactcctgccgttactgccctgtggggcaaggtg

SEQ ID NO: 77 - DNA, 69 bp, Artificial Sequence
  primer
caacctcaaacagacaccatggtgcacctgactcctgagtctgccgttactgccctgtggggcaaggtg

SEQ ID NO: 78 - DNA, 72 bp, Artificial Sequence; identical to SEQ ID NO: 74
  primer

SEQ ID NO: 79 - DNA, 74 bp, Artificial Sequence
  primer
caacctcaaacagacaccatggtgcacctgactcctgagagaagtctgccgttactgccctgtggggcaaggtg

SEQ ID NO: 80 - DNA, 72 bp, Artificial Sequence; identical to SEQ ID NO: 74
  primer

SEQ ID NO: 81 - DNA, 74 bp, Artificial Sequence; identical to SEQ ID NO: 79
  primer